(19) United States
(12) Patent Application Publication
(10) Pub. No.: US 2025/0312544 A1
(43) Pub. Date: Oct. 9, 2025

(54) INTRANASAL DRUG DELIVERY SYSTEM

(71) Applicant: Vistagen Therapeutics, Inc., South San Francisco, CA (US)

(72) Inventors: Louis Monti, Mountain View, CA (US); Reid G. Adler, Alexandria, VA (US); Hemant M. Vishwasrao, Morrisville, NC (US); Ross A. Baker, Bedminster, NJ (US); Mark J. Ginski, Kingsville, MD (US); James A. Sack, Elverson, PA (US)

(21) Appl. No.: 19/172,293

(22) Filed: Apr. 7, 2025

Related U.S. Application Data
(60) Provisional application No. 63/631,389, filed on Apr. 8, 2024.

(51) Int. Cl. A61M 15/08; A61M 11/00; A61M 15/00

(52) U.S. Cl. CPC A61M 15/085; A61M 11/007; A61M 15/009

(57) ABSTRACT

An intranasal drug delivery device for targeting primary, secondary, and tertiary chemosensory receptor areas in the nasal cavity. The intranasal drug delivery device includes an actuator that can be customized with one or more laterally-oriented discharge orifices at varying locations. In one example, the actuator can include an axial outlet at its distal tip for targeting the dorsal nasal recess (olfactory) area as well as additional lateral outlet(s) formed along the sidewall of the actuator for targeting secondary chemosensory receptor sites such as the vomeronasal organ. In addition, the intranasal drug delivery device can include an ergonomic flange component to ensure that users orient the device appropriately with respect to their nostrils during application and thereby significantly improve user compliance.

### CROSS-REFERENCE TO RELATED APPLICATION

This application claims the benefit of U.S. Provisional Patent Application Ser. No. 63/631,389 filed on Apr. 8, 2024 and titled “Intranasal Drug Delivery System”, the disclosure of which is incorporated by reference herein in its entirety.

### TECHNICAL FIELD

The present disclosure relates to an intranasal drug delivery system to administer drugs, particularly pherines, to multiple areas of olfactory chemosensory epithelium in the nasal cavity. Pherines are agonists to receptors on dendrites of nasal chemosensory neurons (NCNs) contained primarily in the mucous layer in the dorsal recess of the nasal cavity and vomeronasal organ. The device will also provide an ergonomic advantage for patient self-administration.

### BACKGROUND

Intranasal drug administration is commonly used to treat local disorders of the nasal passages, such as congestion, rhinitis, sinusitis, and related allergic conditions. Moreover, intranasal drug delivery is a useful and reliable alternative to the oral and parenteral routes for systemic administration of drugs and vaccines. Additionally, it is effective in the administration to the brain (that is, axonal transport, and ophthalmic veins to cavernous sinus) of certain CNS products to circumvent the normally difficult to penetrate blood-brain barrier (BBB). A diverse range of drugs, including corticosteroids, antihistamines, anticholinergics, vaccines, vasoconstrictors, insulin, parathyroid hormone, and opioid antagonists (for example, naloxone), are commonly administered systemically through the intranasal route. See, for example, Thorat, “Formulation and Product Development of Nasal Spray: An Overview,” Sch. J. App. Med. Sci. 4(8D):2976-2985 (2016); Hallschmid, “Intranasal Insulin,” J Neuroendocrinology 33:e 12934, 1-13 (2021); Pearson et al., “Nasal administration and plasma pharmacokinetics of parathyroid hormone,” Pharmaceutics 11(265):1-17 (2019).

Drugs formulated in an appropriate delivery vehicle, such as an aerosol, fluid, or powder dosage form, are administered as nasal sprays in a relatively noninvasive manner and offer a rapid onset of drug action. Because nasal spray devices are easy to carry, simple to use, and self-administrable, they have a high compliance rate and are widely utilized in various prescription and over-the-counter products for drugs taken at regular intervals or on an as-needed (pro re nata or “PRN”) basis.

In practice, the actuator of a conventional nasal spray device is typically inserted through the nostril into the nasal vestibule of a person and advanced past the internal nasal valve. For example, see A therapeutic drug product is then sprayed into the anterior respiratory region of the nasal cavity through a discharge orifice that typically is present at the uppermost (distal) tip of the actuator. For many therapeutic purposes, a broad distribution of the drug on the mucosal surfaces appears desirable for drugs and vaccines intended either for local action or systemic absorption. For example, in the context of general nasal administration, the distribution of a drug to multiple anatomical sites in the nasal cavity is intended, that is, to the anterior, medial, posterior respiratory region, turbinates, and sinuses, and the dorsal nasal recess.

Delivery to these varied and diverse anatomical areas is considered desirable for drugs intended for local action and local effect in the nasal passages, systemic absorption, or axonal transport via the trigeminal nerve, and ophthalmic veins to cavernous sinus blood flow. Accordingly, conventional intranasal delivery devices generally direct medications to the septum and/or the axially oriented upward and posteriorly located regions of the nasal cavity, including the sinuses.

In recent years, unique, odorless neurosteroid drugs have been synthesized to represent a new class of drugs called “pherines” (also called vomeropherines). Low microgram level doses of pherines administered intranasally in spray form have a selective binding affinity as agonists to receptors on dendrites of human nasal chemosensory neurons (NCNs) and rapidly activate specific olfactory bulb-to-brain (or nose-to-brain) neurocircuits. Notably, the NCNs initiate the activation of such basal and anterior brain circuits in brain structures such as the amygdala, hypothalamus and hippocampus, but the compounds themselves do not enter the brain by axonal transport or penetrate through the epithelial cell layer from which the thin mucous layer lining the nasal cavity is produced.

It has been shown that the NCNs in the olfactory epithelium in the dorsal cleft and VNO are responsive to stimulation by pherines. Stimulation of these NCNs can influence autonomic nervous system function, memory and hypothalamus-pituitary function, among other human neurological systems, and also influence behavior. Studies of the human olfactory epithelium in the area of the dorsal nasal cleft and VNO have shown that specific neurons extend to different regions of the basal forebrain via the olfactory bulbs. Collectively, stimulation of the NCNs causes transmission of signals directly to the various brain structures including the amygdala, hypothalamus and hippocampus, which control endocrine function, behavior and emotional responses and memory.

It is believed that subgroups of NCNs activated by pherines can trigger subgroups of microcircuits (also known as glomeruli) in the olfactory bulbs (OBs) known to project via short, oligosynaptic neural circuits to the amygdala, hypothalamus, and entorhinal area/hippocampus. It is proposed and understood herein that in humans, neural inputs triggered by pherines in NCNs activate subgroups of OB neurons that project to neurons in the centro-lateral (CeL) and centro-medial (CeM) amygdala with downstream effects mediating behavioral homeostasis and physiologic homeostasis. As noted, the pherines' differentiated mechanism of action does not involve systemic absorption or binding to steroidal hormone receptors, or direct uptake into the brain. Accordingly, such compounds do not reach or activate abuse-related (for example, opiate) receptors in the CNS, which contributes to a favorable safety profile in clinical trials for all pherine drugs conducted to date.

Thus far, at least five different pherines have been clinically tested with positive signals in exploratory studies across at least six different potential therapeutic indications. U.S. patents related to pherines include, for example, U.S. Pat. No. 5,783,571 (estrenes), U.S. Pat. No. 5,883,087 (androstanes), U.S. Pat. No. 5,563,131 (pregnanes), U.S. Pat. No. 5,792,757 (19-norpregnanes), U.S. Pat. No. 5,994,333 (cholanes), and U.S. Pat. No. 5,922,699 (19-norcholanes), while more recent patents include, for example, U.S. Pat. No. 8,309,539 (“Acute treatment of social phobia”), U.S. Pat. No. 8,431,559 (“Treatment of hot flashes”), U.S. Pat. No. 10,322,138 (“Treatment of depressive disorders”), and U.S. Pat. No. 11,419,881 (“Treatment of migraine”). Recent presentations/articles on pherines include, for example, Liebowitz, et al., “Top-Line Results from Phase 3 PALISADE-2 Trial of Fasedienol (PH94B) Nasal Spray in Social Anxiety Disorder, presented at the Anxiety and Depression Association of America Conference, Apr. 11-14, 2024, Boston, Massachusetts (forthcoming); and Monti, et al., “A Placebo Controlled Trial of PH10: Test of a Rapidly Acting Intranasally Administered Antidepressant,” Brit. J. Pharm. and Med. Res. 4(06):2157-2168 (2019).

However, conventional nasal spray devices may not optimally and concurrently deliver pherine drugs to their most appropriate target areas where the dendrites of NCNs are preferentially located in the mucous layer coating the olfactory epithelium lining the nasal cavity. Accordingly, there is a need in the art for a nasal delivery device and methods of its use that address such shortcomings.

### SUMMARY

In one aspect, a method of delivering drugs to multiple regions of a subject's nasal cavity including the olfactory cleft and the vomeronasal organ (VNO) is disclosed. The method includes a step of inserting a distal end of an actuator of a delivery device through a first nostril and into a first nasal cavity of a person until: (a) a distal orifice formed at an outermost tip of the distal end is between about 35 mm and 45 mm from the first nostril; and (b) a first lateral orifice formed in a portion of a first portion of a sidewall of the actuator is between about 15 mm and 25 mm from the first nostril.

In another aspect, a method of delivering drugs to multiple regions of a nasal cavity including the olfactory cleft and the vomeronasal organ (VNO) is disclosed. The method includes a step of inserting a distal end of an actuator of a delivery device through a first nostril and into a first nasal cavity of a person until: (a) a distal orifice formed at an outermost tip of the distal end is between about 5 mm and 15 mm from an olfactory cleft region of the first nasal cavity; and (b) a first lateral orifice formed in a portion of a sidewall of the actuator is between about 0.1 mm and 2 mm from the VP.

In another aspect, a method of delivering drugs to multiple regions of a nasal cavity including the olfactory cleft and the vomeronasal organ (VNO) is disclosed. The method includes a step of inserting a distal end of an actuator of a delivery device through a first nostril and into a first nasal cavity of a person until: (a) a distal orifice formed at an outermost tip of the distal end is adjacent to a superior turbinate of the first nasal cavity; and (b) a first lateral orifice formed in a first portion of a sidewall of the actuator is oriented in a substantially medial direction, and faces toward a portion of nasal septum of the first nasal cavity adjacent to a nasal valve.

In another aspect, a method of delivering pherines to multiple regions of a nasal cavity including the olfactory cleft and the vomeronasal organ (VNO) is disclosed. The method includes a step of delivering, to the VNO, a first metered dose of a first pherine compound through a first lateral orifice provided in an actuator body of a delivery device, the first pherine compound exiting the first lateral orifice as a mist having an impact pressure on the epithelial layer below the activation threshold of a significant percentage of the high threshold mechanoreceptors.

In another aspect, a method of delivering drugs to multiple regions of a nasal cavity including the vomeronasal organ (VNO) is disclosed. The method includes a step of delivering a first metered dose with an impact pressure of no more than about 0.8 Pascals via a lateral orifice provided in an actuator body of a delivery device to the VNO of a first nasal cavity, thereby covering surfaces of the vomeronasal organ of the first nasal cavity while preventing activation of a majority of high threshold trigeminal mechanoreceptors in the first nasal cavity.

In another aspect, a method for treating a neuropsychiatric disorder is disclosed. The method includes a step of intranasally administering to an individual in need thereof an effective dose of a pherine compound to the nasal cleft and the vomeronasal organ (VNO) with the delivery pressure.

In another aspect, an actuator for an intranasal drug delivery device is disclosed. The actuator includes: a base portion that is in fluid communication with a reservoir of the intranasal drug delivery device; an axially-oriented distal orifice provided in a distal tip portion of the actuator that is in fluid communication with an environment external to the interior chamber; and a first lateral orifice formed through a thickness of a first portion of a sidewall of the actuator, where the first lateral orifice is also in fluid communication with the environment external to the interior chamber.

In another aspect, an intranasal nasal drug delivery device is disclosed. The intranasal drug delivery device includes: a reservoir that includes a pherine composition selected from the group consisting of one or more of fasedienol, itruvone, PH80, PH15, and PH284; and an actuator in fluid communication with the pherine composition. The actuator includes an actuator body comprising of a tubular sidewall extending from a base portion to a distal tip portion. In addition, the distal tip portion includes a distal orifice configured to direct a portion of the pherine composition onto nasal chemosensory receptors associated with the mucosa in the olfactory cleft, and the tubular sidewall includes a lateral orifice configured to direct a portion of the pherine composition onto nasal chemosensory receptors associated with the vomeronasal organ (VNO).

In another aspect, an actuator assembly for an intranasal drug delivery device is disclosed. The actuator assembly includes: a first actuator including a first axially-oriented distal orifice provided in a first distal tip portion of the first actuator, and a first lateral orifice formed through a first thickness of a first sidewall; and a second actuator including a second axially-oriented distal orifice provided in a second distal tip portion of the second actuator, and a second lateral orifice formed through a second thickness of a second sidewall.

In another aspect, by utilization of the disclosed nasal spray devices, the present disclosure contemplates ready-for-administration pherine drug compositions in the reservoir of the device.

In another aspect, the disclosure provides a method of delivering a prophylactic or therapeutic nasal spray to the olfactory chemosensory mucosa, comprising the administration of a spray of pharmaceutical substance with the intranasal drug delivery device.

The disclosure further provides a package or kit that combines a ready-for-administration nasal spray device together with appropriate pharmaceutical product packaging, labeling, and instructions for its use.

In another aspect, as described in more detail below, the disclosure provides an actuator for a nasal spray device, including multiple discharge orifices, that is capable of controlling both the directionality and impact pressure of a nasal spray to optimally deliver a pherine drug to at least the primary and secondary target areas of the olfactory chemosensory epithelium and, preferably, also to the tertiary target areas of the olfactory chemosensory epithelium in the nasal cavity.

In another aspect, the disclosure provides a nasal delivery system for the therapeutic administration of pherine compounds that is improved and optimized relative to conventional nasal spray devices for the various reasons described in this specification. These reasons include the directed release of a plurality of nasal spray plumes to preferentially target therapeutically important areas of olfactory chemosensory epithelium that have an enriched population of nasal chemosensory receptors, modulation of the impact pressure of pherines on their target areas to reduce or avoid the concurrent activation of trigeminal nociceptors, form factors that improve human subject compliance and other features described in this specification.

In different embodiments, the disclosure also provides for a therapeutic device for treating disorders, comprising an intranasal spray device with an actuator configured with two orifices for directing delivery of a pherine-based formulation to both an olfactory cleft and a vomeronasal organ in a subject's nasal cavity.

In different embodiments, the disclosure also provides for a kit of parts for treatment of one or more disorders, the kit comprising: a vial including a pherine-based formulation; and an actuator in fluid communication with the vial, the actuator including two orifices, where the actuator is configured to deliver a first metered dose of the pherine-based formulation to a vomeronasal organ in a subject's nasal cavity, and a second metered dose of the pherine-based formulation to an olfactory cleft in the subject's nasal cavity.

In different embodiments, the disclosure also provides for a kit that includes at least one pherine in a labeled package, wherein application of the at least one pherine occurs by an intranasal spray device inserted into a nasal cavity, and the label on the package indicates that the at least one pherine can be used in treatment of at least one disorder.

Other systems, methods, features, and advantages of the embodiments will be, or will become, apparent to one of ordinary skill in the art upon examination of the following figures and detailed description. It is intended that all such additional systems, methods, features, and advantages be included within this description and this summary, be within the scope of the embodiments and be protected by the following claims. All patents, articles, web pages, and other documents referred to herein are incorporated by reference in their entirety.

### BRIEF DESCRIPTION OF THE DRAWINGS

The embodiments can be better understood with reference to the following drawings and description. The components in the figures are not necessarily to scale, emphasis instead being placed upon illustrating the principles of the embodiments. Moreover, in the figures, like reference numerals designate corresponding parts throughout the different views.

FIG. 1A and FIG. 1B are two views of the lateral wall of the nasal cavity and the septal wall, respectively, showing the distribution of the chemosensory olfactory epithelium contemplated as targets for pherine administration and chemosensory olfactory neurons as conduits for direct to brain transport of other drugs, according to an embodiment;

FIG. 2 is a schematic cutaway view showing an example application of an intranasal drug delivery device by insertion into a human nostril, according to an embodiment;

FIG. 3 is a schematic side view of an intranasal drug delivery device, according to an embodiment;

FIG. 4 is a schematic isometric view of the intranasal drug delivery device, according to an embodiment;

FIG. 5 is a top-down view of a flange component of the intranasal drug delivery device for guiding users during insertion of the actuator into the nostril, according to an embodiment;

FIG. 6 is an isolated side view of an actuator for the intranasal drug delivery device including two laterally-oriented outlets on opposite sides of the actuator, according to an embodiment;

FIG. 7 is an isolated top-down view of an actuator for the intranasal drug delivery device, according to an embodiment;

FIG. 8A depicts an example of drug dispersion as performed by the intranasal drug delivery device via two discharge orifices, according to an embodiment;

FIGS. 8B and 8C present two examples by which the distribution cone angle can be preferably modulated to ensure application of the drug at the target region, according to an embodiment;

FIG. 9 is an isolated side view of an actuator for the intranasal drug delivery device including three laterally-oriented outlets on the same side of the actuator, according to an embodiment;

FIG. 10 is an isometric view of an actuator and flange for the intranasal drug delivery device where the actuator includes three laterally-oriented outlets on the same side of the actuator, as well as a plurality of laterally-oriented outlets on a different side of the actuator, according to an embodiment;

FIG. 11 is a schematic view of the intranasal drug delivery device as it releases multiple streams of a dose of a pharmaceutical substance into a nasal cavity, whereby each stream is associated with substantially similar impact pressure levels, according to an embodiment;

FIG. 12 depicts an example of an intranasal drug delivery device including two discrete actuators for simultaneous delivery of drug to both nostrils, each configured to release drug product through both lateral and distal discharge orifices simultaneously into both nostril, according to an embodiment;

FIG. 13 is a schematic “exploded” view of an interior of one compartment of a nasal cavity for a nose, according to an embodiment;

FIGS. 14A and 14B depict an example of an actuator that includes a recessed portion in which a lateral orifice is provided, according to an embodiment;

FIG. 15A shows an example of an actuator that includes a substantially oval or elliptical or otherwise rounded and elongated cross-sectional shape, according to an embodiment;

FIG. 15B shows an example of an actuator that includes a substantially oval or elliptical or otherwise rounded and elongated cross-sectional shape as well as two recessed portions formed on opposite sides of the actuator body, according to an embodiment;

FIGS. 16A and 16B are schematic diagrams showing an example of an intranasal drug spray delivery device with a D-shaped actuator for insertion in both a left nostril and a right nostril, according to an embodiment;

FIG. 17 is a schematic diagram showing a coronal view of the nose region with an intranasal spray device, according to an embodiment.

FIG. 18 is a schematic view of an intranasal spray device with an actuator that includes a materials gradient of elasticity, and the curving of the upper portion of the actuator upon insertion in a nasal cavity, according to an embodiment

FIG. 19 is a schematic view of an actuator that includes two orifices, each with a different spray nozzle configuration, according to an embodiment;

FIGS. 20A, 20B, 21A, 21B, 21C, 21D, and 21E depict examples of micro-pore configurations that can be included in the lateral orifice to generate a mist, according to an embodiment;

FIGS. 22A, 22B, and 22C show three examples of lateral orifices that each include different channel shapes and spray orientations, according to an embodiment;

FIG. 23 shows an example of an actuator with an elliptical or oval-shaped lateral orifice, according to an embodiment;

FIG. 24 is another example of an intranasal spray device with a D-shaped actuator for insertion in both a left nostril and a right nostril that further includes a recessed portion and a diagonally oriented channel, according to an embodiment;

FIG. 25 is a flow chart depicting a process of delivering drugs to multiple regions of a subject's nasal cavity including the olfactory cleft and the vomeronasal organ (VNO), according to an embodiment;

FIG. 26 is a flow chart depicting a process of delivering drugs to multiple regions of a subject's nasal cavity including the olfactory cleft and the vomeronasal organ (VNO), according to an embodiment;

FIG. 27 is a flow chart depicting a process of delivering drugs to multiple regions of a subject's nasal cavity including the olfactory cleft and the vomeronasal organ (VNO), according to an embodiment;

FIG. 28 is a schematic cross-sectional view of an intranasal spray device revealing components of a pump assembly, according to an embodiment;

FIG. 29 is a cross-sectional view of a portion of the pump assembly, according to an embodiment; and

FIGS. 30, 31, and 32 are magnified views of a portion of the intranasal spray device showing a sequence in which actuation is performed, according to an embodiment.

### DETAILED DESCRIPTION

Over the past decade, the intranasal (IN) drug delivery route has gained interest in research and development and clinical applications for various types of medications and disorders. Nasal drug delivery systems are known to offer effective therapeutic applications locally, systemically, and directly to the central nervous system by avoiding the blood-brain barrier, in some instances at lower doses and with reduced side effects compared to other drug delivery systems. However, delivering particular classes of drugs, for example, pherines, to physiological and anatomically discrete regions in the olfactory epithelium of the nasal cavity, has remained challenging due in part to the narrow geometry of the nasal cavity and because currently available actuator devices primarily service target sites that are upwardly and posteriorly relative to distal ends of the actuator section of the delivery device.

Intranasal drug administration has historically been used to topically and locally treat symptoms of sinonasal conditions, such as chronic rhinosinusitis (CRS), by delivering drugs directly to sinuses and the opening of the sinuses in the turbinates via IN squeeze bottles, spray pumps, powered nebulizers, and breath-powered bidirectional nasal devices. More recently, the trans-cribriform delivery route has become more prominent as an alternative approach to delivering certain drugs to where direct penetration into the brain is required for therapeutic activity. As discussed herein, the present disclosure contemplates the optimized delivery of pherine to avoid any systemic update or direct transport into the brain.

Despite these advances, effective deposition of IN drugs to the dorsal olfactory cleft (OC) remains challenging due to its anatomic seclusion, as well as variations in sinonasal anatomy, devices, drug formulations and administration techniques. For example, while one of the primary functions of the anterior portion of the nasal cavity is to filter out inhaled particles, it also prevents effective delivery of drugs in particular form to the OC and VNO. Common anatomic variations of the middle turbinate (MT), such as concha bullosa, concha lamella and paradoxical middle turbinates, can also serve as obstructions preventing therapeutic particle distribution to the OC.

To overcome these challenges and enable a more targeted and controlled delivery of compounds, particularly pherine drugs, to predetermined sites within the nasal cavity, the proposed actuator apparatus, its cap and actuator, and associated delivery devices, systems, and methods, include provisions by which an intranasal delivery device can, upon insertion into the nasal cavity of a person, deliver a pharmaceutical compound to the preferred target areas that are the most relevant and appropriate for a given therapeutic product, particularly pherines.

As will be described below, the disclosed devices offer improved targeted application of IN drugs to the OC as well as other regions where NCNs are primarily present such as the vomeronasal organ (VNO). The proposed embodiments improve drug delivery to the olfactory chemosensory epithelium by the inclusion of actuator features that complement the spatial relationships of human sinonasal structures in order to preferentially target NCNs by positioning discharge nozzles along the actuator in accordance with varied spatial distances, geometries, and angles associated with the relative locations of OC and VNO in different people.

The present embodiments generally relate to the delivery of therapeutic nasal sprays to multiple predetermined targets of olfactory chemosensory epithelium in the nasal cavity, by which a therapeutic product is released from multiple discharge orifices in the body of the device's actuator to reach olfactory chemosensory epithelium locations in the olfactory cleft and, optionally, also nearby olfactory areas, while also delivering the drug in a radially outward direction to the chemosensory nasal structures in the septal wall of the nose adjacent to the lateral portion of the actuator. In another embodiment appropriate for drugs other than pherines, the device can also preferentially direct the drug to target non-chemosensory epithelium located on the lateral (or outside wall of the nostril).

In other embodiments and for some therapeutic purposes, various embodiments of the device may be used to preferentially deliver a drug other than pherines to the mucosal lining of the nasal turbinates located on the lateral wall of the nasal cavity, as discussed below.

In the description that follows, Part I discusses the olfactory chemosensory epithelium, Part II discusses certain human factor challenges to the effective use of conventional nasal spray devices, particularly when administering pherines, Part III discusses certain trigeminal nerve nociceptors also present in the olfactory chemosensory epithelium, Part IV describes an illustrative use of an embodiment of the proposed intranasal spray device, Part V presents various illustrative embodiments of the structural features of the intranasal spray device according to the present disclosure, Part VI describes some considerations for drug delivery optimization as related to actuator size and discharge orifice characteristics, Part VII discusses possible variations in the arrangement and/or patterns of the laterally-situated discharge orifices, Part VIII describes the modulation of discharge pressure associated with each orifice in order to equalize impact pressures, Part IX discusses an embodiment in which two actuators are formed on the intranasal spray device, Part X discusses further actuator embodiments that can fine-tune delivery to selected target regions, Part XI discusses examples of various orifice configurations that can be used to achieve the desired range and pressures, Part XII discusses some processes, systems, and device features that can be employed by the proposed embodiments, and Part XIII discusses examples of a flow pathway and pump assembly for the proposed device.

### I. Olfactory Chemosensory Neurons and the Olfactory Chemosensory Epithelium

As a general matter, the apical membrane of olfactory chemosensory neurons is provided with olfactory cilia that project into a thin mucous layer that is about 200 μm thick and covers the nasal epithelium. Diverse chemical compounds, including pherines, bind to and activate olfactory chemosensory receptors present in the membrane of these cilia. The axons of the olfactory chemosensory neurons form the olfactory nerve (Cranial Nerve I) and connect the olfactory neurons with the olfactory bulb, which in turn extends neural connections to other specialized regions of the brain (limbic amygdala, hypothalamus, hippocampus, olfactory cortex). Collectively, these neuronal components provide the sense of smell.

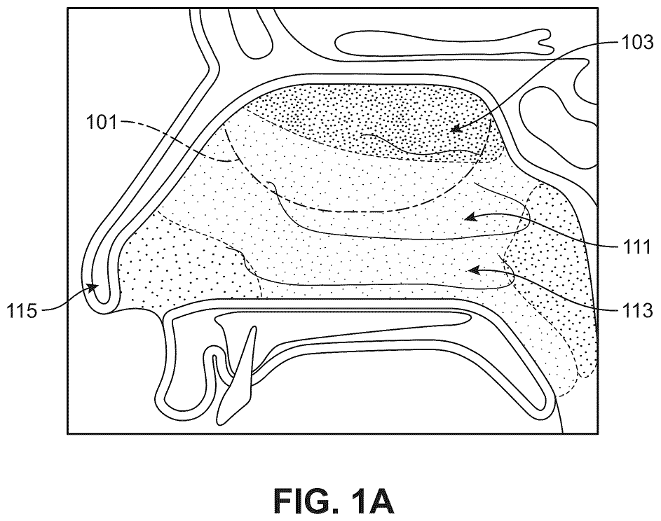

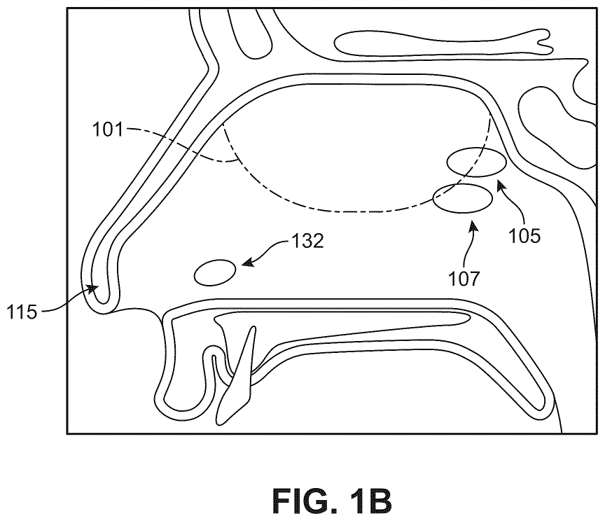

The areas of nasal epithelium where these olfactory chemosensory receptors are found are referred to as the olfactory chemosensory epithelium, although they are also generally considered to be part of the nasal respiratory epithelium. With reference to the schematic internal view of a human nasal cavity depicted in FIGS. 1A and 1B, the olfactory chemosensory epithelium is primarily located in four anatomically distinct areas of the nasal epithelium, each area being an appropriate and preferred target for nasally-administered therapeutics products that selectively activate nasal chemosensory neurons, as discussed above, particularly those containing pherine drugs. It is to be understood that each of these four areas is spaced apart and distinct from the others. In addition, these four areas are present in each of the left-side nasal cavity and right-side nasal cavity, although only the left-side nasal cavity is shown in FIGS. 1A and 1B.

For purposes of simplicity, throughout this description, the term “first nasal cavity” or “second nasal cavity” refers to one of the two compartments of the human nasal cavity, which is anatomically divided into two compartments by the nasal septum (or simply “septum”). The two compartments collectively form the human nasal cavity. Similarly, references to a “right-side” or “right” nasal cavity, or “left-side” or “left” nasal cavity each refer to one of these two compartments of the nasal cavity.

With respect to conventional nasal sprays and particular therapeutic products, there can be a substantial mismatch between, on the one hand, optimizing the delivery of the active pharmaceutical ingredient to the olfactory cleft and, on the other hand, the shape of the expanding plumes generated by mechanical nasal spray pumps, pressurized metered dose inhaler (pMDI)'s and nebulizers. This is because of the gradually constricting dimension of the nasal vestibule, the narrowing barrier of the nasal valve region, and the complex slit-like labyrinthine geometry of the passageway between the nasal valve and the olfactory cleft.

For example, standard conical spray plumes of about 60 degrees typically have a diameter of 2 cm at a distance of only 1 cm from the aperture of the spray nozzle, and at 3 cm from the tip the diameter is greater than 3 cm. Thus, even if a standard spray tip is inserted as much as 10-15 mm into the ellipsoidal-shaped vestibule of the nose there is an obvious mismatch between the dimensions of the narrow nasal valve region and the expanding circular spray plume. The drug particles located primarily in the periphery of the plume will impinge in the non-ciliated mucosal walls of the nasal vestibule, anterior to the valve. Particles that pass beyond the nasal valve will do so primarily in the lower (wider) part of the nasal valve and, thus, will tend to pass along primarily to the lower part of the nasal passages. The proposed actuators and accompanying devices significantly improve delivery to the olfactory cleft for the activation of NCNs by delivering drug molecules directly onto the olfactory epithelium. As will be appreciated by a person skilled in the art, such actuator form factors may be used for other kinds of therapeutic products intended for N2B transport so long as their target areas include the olfactory epithelium described in this specification and their formulations and delivery pressures are consistent with N2B products. For purposes of this application, “delivery pressure”, also referred to as “impact pressure”, refers to the pressure value or pressure range of the device-emitted spray of formulation/drug as it arrives at its target and impacts onto a surface/region of the nasal cavity (e.g., surfaces of the olfactory chemosensory epithelium). The impact pressure is expressed in Pascals. Impact pressure is directly related to the force of the impact and the area over which that force is distributed (Pressure=Force/Area). Thus, references to the “force of impact” can relate to a directionality of the pressure that is applied.

FIG. 1A depicts a lateral wall view of the nasal cavity and FIG. 1B depicts a medial (or septal) side view of the nasal cavity. While the human nasal cavity is highly variable among individuals, it does retain several key features. The nasal cavity is nominally symmetric and is separated into two distinct air passages by a vertical thin wall called the nasal septum. The top part of the nasal cavity is formed by bones and cartilage and is tent-shaped. The floor of the nasal cavity is formed by the palate, which separates the nasal and oral cavities and extends horizontally toward the posterior of the skull.

At the nostril, the entrance to the nose, the shape of the nasal cavity varies between circular and oval. As the nasal passage bends and constricts, the cross-sectional shape of the cavity becomes more elongated and triangular with the narrowest dimensions of the triangle lying superiorly. This narrow constriction is termed the nasal valve region and is located approximately 2-3 cm from the nostril, with a mean cross-sectional area of only about 0.5-0.6 cm2 on each side. The nasal valve is the narrowest segment of the entire respiratory tract and accounting for as much as about 50-75% of the total airway resistance. It represents an often-underestimated hurdle for nasal drug delivery.

As reflected in FIG. 1A, there are three shelf-like structures on the lateral wall known as turbinates or conchae, which serve to increase the surface area exposed to the air, thus increasing heat and moisture exchange. The grooved space below each turbinate is referred to as a meatus. The turbinates and meatuses are each labeled superior, middle, or inferior. In the superior region of the nasal cavity, the walls are covered by olfactory mucosa even though much of this distributed mucosal layer does not contain NCNs, which are found preferentially in the dorsal cleft and VNO. For most humans, the minimum cross-sectional area CSA (MCA) of the nasal cavity occurs in a region about 20-35 mm from the tip of the nostril, where the corresponding airway narrowing is called the nasal valve. In general, the nasal valve occurs at a location roughly about 24 mm from the nostril. The CSA then increases as expected in the region of the turbinates (around about 35-75 mm from the nostril).

The olfactory epithelium (OE), which lines the surface of the dorsal recess of the human nasal cavity, is known to be a portal for external chemo-signals (including olfactory stimuli and odorless external chemosignals) carried by air during the respiratory cycle, that activate the olfactory cortex and limbic system structures via the olfactory nerves. Thus, in different embodiments, the primary target area of the olfactory chemosensory epithelium in pherine administration is the dorsal nasal recess 101 (also referred to herein as the olfactory cleft region), which is believed to contain about 80 to 90% percent of a person's olfactory chemosensory receptors. This area of olfactory chemosensory epithelium spans the dorsal nasal recess 101 from the upper portion of a superior turbinate 103 (represented in the drawing by a curving line in the respiratory mucosa) on each lateral wall of the nose to both sides of septum 115.

There is also a secondary target area of olfactory chemosensory epithelium for pherine administration, referred to as a vomeronasal organ (VNO) 132. The VNO 132 is believed to contain about 10% of a person's olfactory chemosensory neurons. This area of olfactory chemosensory epithelium is a recessed structure in the lining of the nasal mucosa with a central depression called the vomeronasal pit, and is found in the septal wall of the anterior olfactory portion of the nasal cavity. See, for example, Moran, et al., “The vomeronasal (Jacobson's) organ in man: ultrastructure and frequency of occurrence,” J. Steroid Biochem. Molec. Biol. 39(4B)545-552 (1991). Stensaas et al., “Ultrastructure of the human vomeronasal organ”, J. Steroid Biochem. Mol. Biol., vol. 39(4), pp. 553-560(1991), D′Aniello, et al. 2017 Frontiers in Neuroanatomy); Stoyanov et al., “Chapter 20—The vomeronasal organ: History, development, morphology, and functional neuroanatomy,” in Handbook of Clinical Neurology, Vol. 182:283-291 (2021); and Monti-Bloch et al., “The Human Vomeronasal System: A Review”, Ann. N.Y. Acad. Sci., vol. 855, pp. 373-389(1998).

Additionally, there are two tertiary areas (or subsystems) of olfactory chemosensory epithelium: Massaera's organ 107 and Grüneberg's organ 105, which together are believed to contain up to about 5% of a person's olfactory chemosensory neurons and provide tertiary targets for pherine administration. These latter two areas of olfactory chemosensory epithelium are found on the dorsal and posterior olfactory region of the nasal cavity. See, Salazar et al., “The nasal cavity and its olfactory sensor territories,” Frontiers in Neuroanatomy 9:1-3 (2015). Thus, outside of the olfactory mucosal region, there are three additional areas in the nasal cavity that are desirable targets for drug delivery, particularly for administering pherines for chemosensory activation.

Although the description herein will provide description focused on the VNO 132 as a preferred additional target for pherine drug delivery together with the olfactory cleft, it should be understood that, in different embodiments, either or both of the Massaera's organ 107 and Grüneberg's organ 105 can similarly be targeted using the proposed devices and techniques.

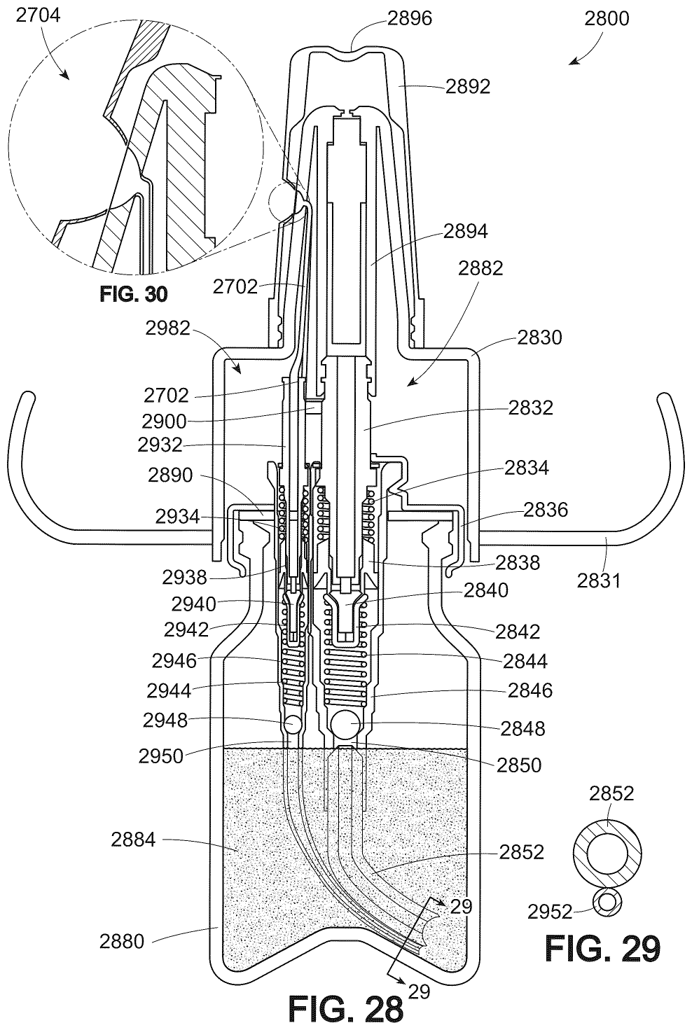

Furthermore, in the following discussion, a person skilled in the art is presumed to be familiar with the architecture, components, and form factors for a typical nasal spray device, including, for example, a drug reservoir, actuator, discharge orifices of various shapes and dimensions, and the channels and conduits that interconnect such components. Drawings of one embodiment of a nasal spray device are provided in FIGS. 28-32 for the purpose of reference to the reader.

### II. Consideration of Human Factors

Although nasal spray devices have been proposed for pharmaceutical use across many different therapeutic applications, complicating the use of nasal spray devices for such purposes is the widely variable shapes and sizes of people's noses and nasal cavities. It is well known that nasal cavity geometries can have significant variations between individuals based on factors such as age, gender and ethnicity, which can create major obstacles for effective nasal drug targeting and deposition. See Warnken, et al., “Personalized Medicine in Nasal Delivery: The Use of Patient-Specific Administration Parameters To Improve Nasal Drug Targeting Using 3D-Printed Nasal Replica Casts,” Mol. Pharm. 15(4): 1392-1402 (2018). Similarly, a study of ten adult males ranging in age from 30 to 57 years showed a range in nasal airway dimensions from 190 to 260 square cm. Cheng et al., “In Vivo Measurements of Nasal Airway Dimensions and Ultrafine Aerosol Deposition in the Human Nasal and Oral Airways,” J. Aerosol. Sci. 27(5)785-801 (1996); Likus, W et al. Nasal region dimensions in children: a CT study and clinical implications. Biomed Res. Internat. 2014, http://dx.doi.org/10.1155/2014/125810; Zalzal, HG et al. Pediatric anatomy: nose and sinus. Operative techniques in Otolaryngology, 23018, 29:44-50; Yamakawa et al. Auris Nasus Larynx 51 (2024) 917-921.

As discussed below, there is significant variability about where the targets for pherine nasal drug administration are to be found on the canvas of the nasal cavity. Accordingly, wherever the nozzle tip of an actuator might be placed inside a nostril, its relative location inside the nasal cavity will vary from person to person relative to the positions of their olfactory chemosensory epithelium.

Conventional nasal spray devices typically have a single discharge orifice at the distal tip of the actuator. Often, the actuator is about 2.5 cm long and extends from a flange that is depressed by the fingers of a person who actuates the nasal spray device. In use, because the thickness of a user's fingers limits the insertion depth of the actuator, the actuator is typically inserted about 1 to 2 cm, a distance that allows the actuator tip to pass through and release a nasal drug product beyond the internal nasal valve, the narrowest part of the nasal cavity, which is located an average of about 1.3 cm from the anterior nares (also referred to herein as the nostrils, or simply “nares”). The drug is released into the nasal cavity in the form of an expanding spray plume from a single location inside the nostril, which, by design, is also the maximum depth that the tip of the actuator penetrates into the nostril. For some conventional nasal spray devices, having a given actuator length, its distal discharge orifice is likely to be positioned closer to or even beyond the location of the vomeronasal organ in some people.

Accordingly, the anatomy of the nasal cavity requires special consideration when using nasal spray devices to intranasally administer pherine drugs to sufficiently activate olfactory chemosensory neurons and neurocircuitry necessary to achieve therapeutic effects. For example, the relative location of the vomeronasal organ's “pit” from the nares generally varies from about 2 to 2.5 cm, as does its size, and is considered to be approximately in the range of about 3 to 8 mm in diameter. Some studies report even more variability, that is, a range of about 5 to 18 mm in diameter and an average of about 9 mm in distance between the nasal floor and the opening to the vomeronasal duct inside the pit. See, for example, Abolmaali et al., “Imaging of the Human Vomeronasal Duct,” Chem. Senses 26:35-39 (2001). In some people, because of the size of their fingers when resting on the flange of the nasal spray device and their personal nasal anatomy, a substantial portion of a pherine nasal spray plume from a conventional nasal spray device could bypass the vomeronasal organ.

Furthermore, even if the distal opening of the actuator (that is, the distal discharge orifice portion) were introduced only slightly into the nasal vestibule, remaining exterior (anterior) to and below the vomeronasal organ, or if a shorter than conventional length actuator were utilized, a substantial portion of whatever section of the nasal spray plume that contacts the vomeronasal organ may do so tangentially and roll or flow across the vomeronasal pit rather than making contact more orthogonally or at an effective angle as would facilitate delivery of the nasal drug into the opening of the vomeronasal duct. For example, U.S. Pat. No. 8,757,146, is also representative of conventional nasal spray devices and discloses a spray being directed upwardly from the distal outlet of an actuator.

Changing the angle of administration of the actuator to aim the distal discharge orifice toward the vomeronasal organ or widening the spray plume as it is emitted from the distal discharge orifice are not optimal solutions. Even though more of the administered drug thereby might impact the vomeronasal organ, the spray plume also would tend to rebound from side to side within the nasal cavity, coating the nasal turbinates while doing so, which are not a target for pherines drugs, and also losing force before reaching the dorsal recess.

Such limitations with regard to targeting pherine drugs to the vomeronasal organ also arise with the use of conventional actuators that are currently available from various manufacturers such as, for example, Aptar Pharma (https://aptar.com/pharmaceutical/delivery-routes/nasal-system-drug-delivery-oindp/), Bespak by Recipharm (https://www.recipharm.com/sites/recipharm-corp/files/recipharm/recourse/fact-sheet/Nasal_spray_device_Unidose_Xtra.pdf), and others. It is understandable why the functionality of such actuators is not optimal when administering pherine drugs, because conventional nasal spray devices were not designed to treat the vomeronasal organ as a significant target area.

Nonetheless, conventional actuators are generally effective for delivering most nasal drugs because the spray plume originates downstream from typical nasal cavity targets, that is, as shown in FIG. 1A, the turbinates (including superior turbinate 103, middle turbinate 111, and inferior turbinate 113), dorsal recess, and posterior nasal cavity 115, regardless of how deeply the discharge orifice is positioned inside the nostril.

A different device form factor, specifically intended for the administration of pherine drugs to the vomeronasal organ, was suggested by PCT Application WO1997/027887 titled “Device and method for delivery of matter to the vomeronasal organ”. The patent application explains that the outside of the housing should be marked to facilitate alignment by the user of the device. It teaches that the actuator's “delivery outlet” (or discharge orifice) should be positioned “over” the vomeronasal organ, and the nasal spray should be directed laterally from the discharge orifice toward it.

However, while potentially improving pherine drug delivery to the vomeronasal organ, the foregoing device may not optimally deliver pherine drugs to the primary target for olfactory chemosensory neurons located in the dorsal olfactory recess and to the tertiary chemosensory epithelial target areas in the posterior olfactory region of the nasal cavity. Moreover, there may have been limited motivation to develop such a device commercially when some recent articles in the medical literature assert that the vomeronasal organ is vestigial and may have no meaningful function in chemoreception. See, for example, Bruintjes, T., et al., “The clinical significance of the human vomeronasal organ,” Surgical and Radiologic Anatomy 45:457-460 (2023). However, in preferred embodiments of the present disclosure, the vomeronasal organ is considered to be an appropriate target for the NCNs that are activated by pherines.

Given the foregoing considerations of actuator design and length of commercially available nasal spray devices, as well as the diversity of nasal anatomy, if a person were instructed to position a nasal spray actuator so that its tip is adjacent to their vomeronasal organ or to align the actuator with the dorsal recess, most people would be unfamiliar with the relevant nasal anatomy. As a practical matter, given the currently available nasal spray devices, the instructions for the administration of a pherine drug candidate product to participants in an ongoing Phase III clinical trial are for the subjects to position the tip of the actuator pointing toward the center (not the side) of their nose with an angle of about 30 degrees and to insert the actuator until the subject's fingers on the flange touch the rim of their nostril. This guidance helps to target the olfactory chemosensory receptor areas of the vomeronasal organ, to reduce the amount of drug that might otherwise be trapped by the mucosal lining of the nasal turbinates rather than reach the dorsal recess, and to discharge the bulk of the nasal spray from the actuator toward the dorsal recess. However, the present disclosure is directed to improvements in nasal spray delivery systems that will optimize the delivery of pherines to their preferred targets in the olfactory epithelium and reduce human factor variability in drug administration.

The foregoing consideration of human factors and ergonomic design is somewhat further complicated by the need for study participants to hold their breath when receiving a pherine drug dose so that the spray plume is more likely to impact the dorsal nasal recess rather than substantially being inhaled through the nasopharynx into the trachea and the lungs. Thus, simplifying the use of the nasal spray device overall for pherine drug administration is desirable.

It is important to note that previous clinical trials for pherine drug candidates, which utilized conventional nasal spray devices, have, more often than not, successfully met contemplated clinical endpoints. Accordingly, a person skilled in the art should understand that the present disclosure and the embodiments described herein relate to certain form factors and spray parameters for nasal spray device actuators to improve targeting of the olfactory chemosensory epithelium by pherines sprays, as well as to the improvement of device design to alleviate certain human factor concerns. The proposed drug delivery systems offer significant advantages with respect to the therapeutic administration of pherines to the OC and the VNO.

### III. Activation of Trigeminal Nerve Mechanoreceptors

In the mammalian nose, the trigeminal nerve provides a network of nociceptors, which are sensory fibers that respond to various stimuli (nociceptive stimuli) that are potentially damaging to the organism. Such stimuli include pressure, temperature, inflammation, and noxious chemicals. See Puopolo et al., “Nociceptors: The Gateway to Pain,” Reference Module in Neuroscience and Biobehavioral Psychology, 2017.

Together with the NCNs described above, the trigeminal nerve also mediates the detection of chemicals. Some odorants are able to activate the trigeminal system, and vice versa; and some trigeminal odorants (menthol, eucalyptol, camphor, diallyl sulphide, propanol, ethanol, cinnamaldehyde, and capsaicin) activate the olfactory system as well. Although these two systems constitute two separate sensory modalities, in some cases, certain molecules can simultaneously activate both the olfactory and trigeminal odor detection systems. If this happens, the trigeminal nerve signal has been reported to modulate the olfactory system's response to these molecules' odors. Such modulation can comprise a graded reduction of the olfactory signal, such that there is potential for trigeminal agonist exposure to influence olfactory sensory neuron activity. Thus, it can be appreciated that trigeminal activation can alter the overall responses to chemicals even at the earliest stage of the olfactory sensory transduction. This would be undesirable for purposes of the present disclosure.

Pherines are odorless compounds that specifically bind to olfactory chemosensory neurons and are not believed to be detected by the trigeminal chemoreceptors. However, the manner in which a nasal spray impacts the target epithelium has the potential to activate other trigeminal nociceptors, particularly those that respond to pressure. Accordingly, it will be appreciated by persons skilled in the art that trigeminal activation may be detrimental to the brain's processing of olfactory signals activated by pherines.

As described herein, in addition to the enhanced and expanded functionality provided by the disclosed embodiments in directing pherines to primary, secondary and tertiary targets in the olfactory chemosensory epithelium, the proposed embodiments further include provisions for reducing the potential activation of certain trigeminal nerves by the discharged spray. Olfactory nerve axons originating in the olfactory bulb penetrate the cribriform plate and extend downwards on both sides of the olfactory cleft. High-speed and high-pressure/force impaction, locally concentrated anterior drug deposition on the septum, as well as direct physical contact with a nasal spray device tip during actuation, may activate trigeminal nerves and cause mucosal irritation and injury, reducing patient acceptance and compliance. Accordingly, the present disclosure is intended, in certain embodiments, to avoid or reduce the trigeminal nerve activation that may be caused by such factors.

As a general matter, the maxillary nerve, which is a branch of the trigeminal nerve (Cranial Nerve V), contains fibers that also innervate portions of the olfactory chemosensory epithelium and other areas of the nasal respiratory mucosa. Ishimaru, et al., “Topographical differences in the sensitivity of the human nasal mucosa to olfactory and trigeminal stimuli,” Neuroscience Letters 493:136-139 (2011). The apical bodies of these trigeminal fibers have receptors for mechanical, chemical, nociceptive, and thermal stimuli and respond to chemical irritants, pain, and pressure. Schiebe, M., et al., “Intranasal trigeminal sensitivity: measurements before and after nasal surgery,” Eur. Arch. Otorhinolaryngol. 271-87-92 (2014).

Trigeminal receptors are relevant to the present embodiments for several reasons: they are found in portions of the olfactory chemosensory epithelium that are targeted by pherine sprays and are also found in portions of the respiratory epithelium (rather than olfactory chemosensory epithelium) that are touched by pherine sprays. The apical bodies of the trigeminal fibers connect to the semilunar sensory ganglion (also known as the trigeminal ganglion or Gasser ganglion), which in turn communicates with various brain and central nervous system structures. See, for example, Yu, M., et al, “Neuroanatomy, Semilunar Ganglion,” NCBI Bookshelf, StatPearls (Internet, 2023). Moreover, it has been reported that trigeminal nerve stimulation can affect the perception of odor by the olfactory nerve. Cain and Murphy Nature 1980 “Interaction between chemoreceptive modalities of odor and irritation,” Nature 284:285-287 (1980).

Among the sensory neurons, mechanosensory neurons respond to a variety of mechanical stimuli or a range of mechanical forces, and then generate tactile or noxious sensations. Furthermore, it is important to appreciate that mechanoreceptor activation thresholds vary along the nasal cavity. In several studies using tactile and electrical stimulation, the anterior region—particularly the nasal vestibule and septum—exhibited lower thresholds (that is, higher sensitivity) than the posterior region. For example, it has been reported that the nasal vestibule required about half the stimulus intensity needed for the nasal cavum, with significantly greater trigeminal sensitivity anteriorly. The trigeminal nerves also follow a clear sensitivity gradient, with the anterior vestibule most responsive, and the highest negative mucosa potentials occurring in the anterior septum relative to the less responsive olfactory cleft. These results indicate that, for many mechanical stimuli, activation thresholds are lower in anterior nasal regions compared with posterior areas. By and large, the threshold concentrations for trigeminal chemoreception are much higher than those for olfaction or taste. In addition, among trigeminal mechanoreceptors, light, innocuous touches are sensed by low-threshold mechanoreceptors (LTMRs), whereas noxious mechanical stimuli are conducted by mechanosensory nociceptors that include high threshold mechanoreceptors (HTMRs). Nociceptors are uniquely tuned to stimuli that cause damage or threaten to cause damage. High-threshold mechanoreceptors (HTMRs) thereby encompass a category of mechano-nociceptive sensory neurons that are optimally excited by noxious mechanical stimuli. This class of neurons responds exclusively to high-threshold mechanical stimuli and are likely critically important for pain and the experience of discomfort and can trigger protective respiratory reflexes that can interfere with the activation of the olfactory neurons.

With respect to the applications of an intranasal spray to the human nose, it can thereby be appreciated that triggering such HTMRs would be highly undesirable, both reducing a person's likelihood of compliance to any associated drug delivery regimen as well as potentially reducing the efficacy of any such drug. Of particular interest for purposes of the present embodiments are these “high threshold” (that is, requiring a higher energy of activation) trigeminal mechanoreceptors, that is, the HTMRs discussed above. If such receptors are triggered by the force of impact of a pherine nasal spray plume, the trigeminal nerve also sends a signal from the nasal cavity into the brain. However, activation of this additional channel of sensory input at or around the same time as the application of the nasal spray plume to the targeted regions in the nasal cavity is believed to be less than optimal when administering pherines. For example, this additional pathway of activity can cause discomfort or pain and complicate and potentially distract or overload the brain's processing of the olfactory signal created by the detection of the pherine compound, diminishing, inhibiting or potentially overriding the transmission of the NCN's signal into the olfactory bulb and beyond into deeper brain structures, and potentially also a subject's clinical response to the pherines.

Accordingly, in addition to and separate from improvements to the targeted delivery of pherine nasal sprays to the olfactory chemosensory epithelium, the present embodiments relate to devices and methods for their use in which the impact pressure of a pherine nasal spray threshold on the nasal epithelium is below the pressure that otherwise would activate a substantial portion (that is, approximately 20-30%) of such high-threshold nasal trigeminal mechanoreceptors. By limiting activation of high-threshold trigeminal mechanoreceptors to less than about 30%, preferably less than about 20-25%, and in preferably below 20%, 19%, 18%, 17%, 16%, 15%, 14%, 13%, 12%, 11%, 10%, 9%, 8%, 7%, 6%, 5%, 4%, 3%, 2%, 1% and substantially none of the HTMRs, the pherines can be delivered with little to no discomfort for the user, and reduce the likelihood of a diminished response to the pherines by the olfactory system resulting from HTMR activation.

To achieve this goal, in different embodiments, the proposed device is configured to restrict the impact pressure of the spray on the nasal epithelium at any target area so that it is less than or equal to about 0.5 to 3.0 Pa, preferably less than or equal to about 1 to 2.5 Pa and more preferably less than or equal to about 1 Pa, and most preferably less than or equal to about 0.9, 0.8, 0.7, 0.6, 0.5, 0.4, 0.3, 0.2 and 01 Pa. Thus, in different embodiments, the proposed intranasal device can be configured to limit the impact pressures of its spray plume to a value within these ranges. In different embodiments, management of this target pressure is of particular importance because the lateral discharge orifices are likely to be less than about 1 cm from the target vomeronasal epithelium, rather than about 2 to 4 cm from the dorsal nasal recess.

The foregoing controls on the impact pressure of the spray plume further reduce any likelihood that administration of the spray plumes would cause discomfort to a user, while also mitigating the potential activation of a potentially distracting or overriding V cranial nerve input to the brain in a manner that might potentially reduce the effectiveness of a pherine drug. In some preferred embodiments, the lateral “impact pressure” or force from the spray that is produced by the discharge orifice(s) should be lower than the upward impact pressure or force because of the respective travel distances, thereby avoiding triggering a substantial portion of the trigeminal mechanoreceptors. As a general description, the lateral sprays will be in the form of a soft mist rather than a jet. In contrast, the distal spray from the distal discharge orifice will be relatively more of a jet shot because it must travel to reach a farther distance of the olfactory chemosensory epithelium.

### IV. Operational Embodiments of the Intranasal Spray Device

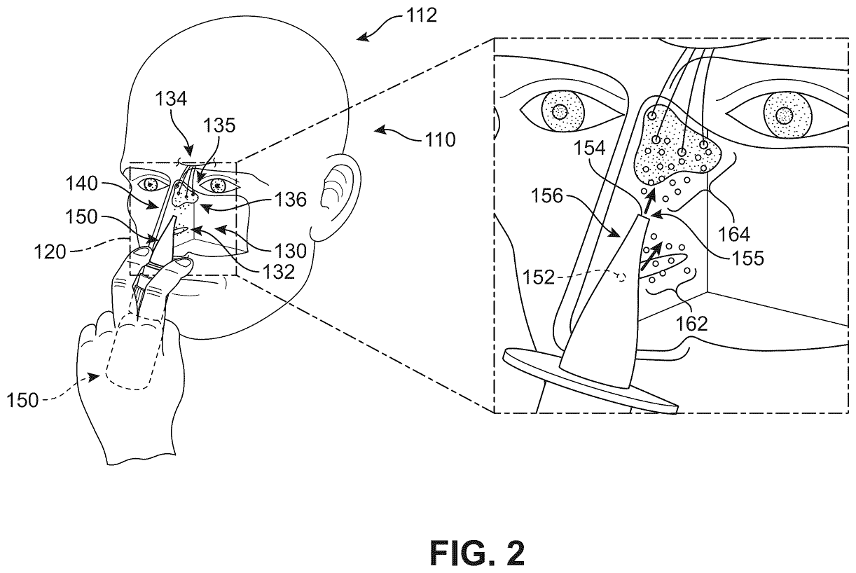

For purposes of further introduction, one example of the proposed embodiments is illustrated in FIG. 2. An isometric view of a head 112 of a first person 110 is depicted in which a first nasal region 120 has been exposed to reveal a first nasal cavity 130 along the left side of the first person 110 (relative to the device user's view).

In FIG. 2, first person 110 is shown during a process by which a pharmaceutical compound is being released into their nasal cavity via an intranasal drug delivery device (“device”) 150. An actuator body (“actuator”) 156 of the device 150 has been gently inserted through a nostril into the anterior and proximal portion of the nasal cavity 130 of their nose 140. The actuator 156 has been guided upward until the actuator 156 is in a pose that corresponds to a distal tip 154 of the actuator 156 being positioned below the olfactory cleft at the roof of the nasal cavity. The actuator 156 can include one or more discharge orifices, or simply “orifices”. For purposes of this application, an orifice refers to a fluid flow path extending through a thickness of the actuator body, where the fluid flow path is defined by an internal chamber having an inlet and an outlet. The orifice can also be referred to interchangeably as a nozzle.

In one example, the distal tip 154 can be positioned so that the spray released through a first (or distal) discharge orifice 155 comes into contact with a region of the olfactory epithelium 136 in the area of an olfactory cleft 135 of nasal cavity 130 where pherine receptors on the dendrites of chemosensory neurons are present. The distal tip 154 can be oriented to provide a distribution of the nasal spray to a first zone of chemosensory olfactory epithelium, which is innervated by chemosensory neurons originating from the olfactory bulb 134. In other words, with this nasal spray device positioned in the nostril, when the first person 110 actuates the device 150, triggering an expelling operation of the drug via the actuator 156, an axially-oriented first discharge orifice 155 provided within the distal tip 154 can direct delivery of a first distribution 164 of the drug into a first region of the olfactory mucosa 134. For clarity, this first region includes olfactory mucosa on both the external (sidewall) and internal (septal) walls of the nasal cavity 130.

As noted earlier, the proposed embodiments can include provisions for drug delivery that target additional secondary regions of chemosensory olfactory epithelium within the nasal cavity 130. For example, chemosensory neurons activated by pherines are also found in the vomeronasal pit on the septum relatively nearer the nares. Targeted delivery to this area can be achieved by including one or more laterally oriented discharge orifices formed along an outermost/external sidewall of the actuator 156. In this case, a second discharge orifice 152 is represented by a dotted line to indicate its position on the posterior side of the actuator in this figure (the side of the actuator facing away from the viewer). Such a dotted line representation has been depicted on the anterior side of the actuator for the reader's convenience, as the opposing side of the actuator is not visible in this perspective.

Through this second discharge orifice 152, a spray plume is emitted during the same drug-expelling operation as described above, and so there is a second distribution 162 of the drug into a second region of chemosensory olfactory epithelium on the nasal septum that is physically spaced apart and anatomically distinguishable from the first region. In the example of FIG. 2, this second region corresponds not to the olfactory cleft region, but instead to the area within the first nasal cavity 130 on the nasal septum where the vomeronasal organ (VNO) 132 is situated.

It can be appreciated that with this arrangement of outlets or discharge orifices from the actuator, the flow of the pharmaceutical compound as it is ejected can be directed toward multiple areas in the nasal cavity. In other words, rather than limiting the application of the drug to the area of olfactory epithelium in the nasal cavity that is adjacent to distal tip 154 of actuator 156, the drug can be applied simultaneously to an anterior portion of the nasal cavity, through a portion of the actuator body sidewall where one or more additional discharge orifices have been formed. For purposes of this description, the use of the term “adjacent” or “near” refers to two structures that are within 0-15 mm of each other. The phrase “directly adjacent” refers to two structures that are within 0-10 mm of each other. In addition, the phrase “abutting” or “abut” refers to two structures that contact one another, while “in close proximity to” refers to structures that are within 0-5 mm of each other.

As will be discussed in greater detail below, and as would be appreciated by persons skilled in the art, physical characteristics associated with the discharge orifice itself, such as discharge orifice size, geometry, shape, and orientation, can be carefully selected to modulate the direction, velocity, and pressure of the spray output dispersed from these laterally oriented discharge orifices. The contributions of various device components to effective delivery are described in greater detail in the technical paper from Aptar Pharma at https://aptar.com/wp-content/uploads/2022/04/Overview-of-Intranasal-drug-administration-using-multi-dose-nasal-spray-pumps.pdf, among others. These include the actuator assembly, dip tube, pump assembly, and other components known to persons skilled in the art.

For purposes of the present disclosure, as discussed below, it is contemplated that two discharge orifices may be provided on opposite sides of the sidewall of actuator 156 so that the VNO 132 will be targeted regardless of which nostril is receiving the administered drug. In this case, a portion of the spray also will be applied to an area on the lateral wall of the nasal cavity that does not preferentially contain chemosensory neurons, where it will cause little or no discomfort but provide no therapeutic benefit. Such a device configuration may facilitate a person's use of the nasal spray device, without requiring special attention to which side and discharge orifice of the actuator should be aligned toward the VNO (that is, on the septal wall).

As will be discussed in further detail below, the proposed embodiments offer significant improvements with regard to conventional nasal spray devices by facilitating a maximized exposure of the drug to the dendrites of nasal chemosensory receptors (NCNs) in the dorsal cleft (dorsal recess) via a spray plume released from its distal (anterior) opening and concurrently or simultaneously also targeting NCNs in the area of the VNO through a lateral opening, thereby promoting a consistent efficacy of the drug delivery operation.

Beyond their role in therapeutic treatment involving the administration of pherines, the olfactory chemosensory neurons located in the nasal cavity provide an alternate pathway for the direct nose-to-brain delivery of drugs into the central nervous system (CNS) without a need for systemic administration. Accordingly, in different embodiments, the proposed devices can be used to administer a pharmaceutical agent to the olfactory chemosensory epithelium that is innervated by the olfactory nerve. As described above, the olfactory neural pathway primarily innervates the olfactory epithelium in the dorsal recess and the vomeronasal pit. Although this route of delivery is not contemplated for pherine drugs, the olfactory neurons that innervate this tissue can provide a direct connection to the cerebrospinal fluid for drugs intended for delivery directly to the brain. As will be understood by persons skilled in the art, applying an appropriate pharmaceutical compound to a tissue innervated by the olfactory nerve thereby can deliver the drug to certain structures and targets within the CNS. After interaction with cell dendrites, the drug moves toward the brain by extracellular and intracellular transport mechanisms and follows the nerve tracts of olfactory cells. Finally, it enters the olfactory bulb and reaches the cerebrospinal fluid (CSF). However, the physicochemical characteristics of the drug, the excipients with which it is formulated and the pressure of the spray exiting the device, as well as the pressure of delivery would need to be considered for such purposes.

### V. Structural Features of the Intranasal Spray Device

Nasal spray device systems generally comprise a formulation, a container, and an actuator. Such devices can rely on the atomization of the formulation to form a plume of droplets as it is emitted from the device. Parameters that can affect the physical characteristics of the plume (for example, droplet size and spray pattern) are known to persons skilled in the art and include, for example, the formulation ingredients, the viscosity and surface tension of the formulation, the design, dimensions, and shape of the actuator, and the technique used to actuate the nasal device. Standard multidose devices include a vial, medication storage unit, or a bottle, and a spray pump with an actuator. In some embodiments, a dip tube goes from the actuator into the formulation to enable it to be expelled up through the actuator when the actuator pump is depressed.

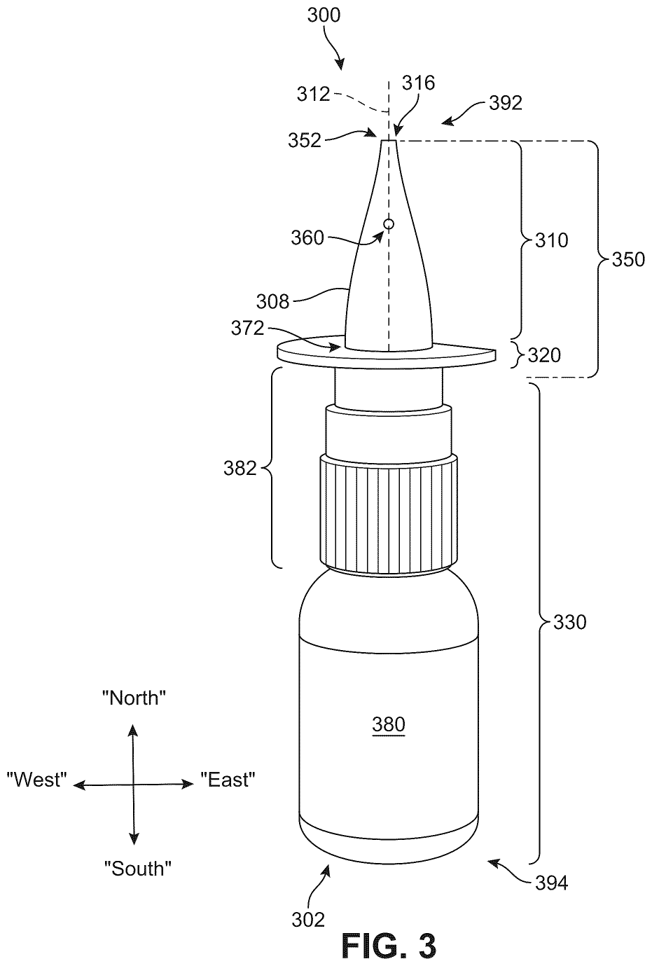

Moving now to FIG. 3, another example of an intranasal spray device (“intranasal device”) 300 that may be capable of simultaneous delivery to first distribution site 162 and second distribution site 164 depicted in FIG. 2 is shown. For purposes of reference, it should be understood that the term “axial” refers to the longitudinal and rectilinear direction of the device's output, such as it is expelled through the primary/distal-most orifice of the device actuator, aligned with a dotted-line longitudinal axis 312 in FIG. 3. Similarly, the term “lateral” refers to the lateral-medial axis and device outputs that are substantially directed to sites from along this axis. In other words, additional orifice(s) formed through the sidewalls of the actuator body of the proposed device will be referred to as laterally oriented orifice(s). In addition, although the term “spray” may be used throughout this disclosure, it is to be understood that the proposed embodiments can be implemented with any suitable intranasal treatment devices that can be used to deliver a range of forms of pharmaceutical compositions, including aerosol, mist, vapor, liquid, solid, powder or semisolid forms. Some examples of pharmaceutical compositions in liquid form include solutions, emulsions, suspensions, colloids, etc. In liquid form, the pharmaceutical composition can be administered as nasal drops or nasal spray. Some examples of pharmaceutical compositions in semi-solid form include ointments, salves, gels, or creams.

For clarity, the description makes reference to distal and proximal directions (or portions) in the context of the intranasal device 300. As used herein, the distal direction is a direction oriented away from a bottom portion 302 of the intranasal device 300 and toward the top of the actuator, while the proximal direction is an opposing direction that is oriented away from the top of the actuator, and toward the bottom portion 302. The proximal and distal directions can also be understood to refer to opposing directions relative to longitudinal axis 312. In addition, the actuator 310 can be understood to have both a proximal and distal direction. Proximal direction can also be referred to as an “upstream” or cranial direction when in the context of a human body, and distal direction can be referred to as “downstream” direction or caudal direction. Thus, the term “longitudinal” as used throughout this detailed description and in the claims refers to a direction extending between a proximal end 394 and a distal end 392 of the intranasal device 300. Additionally, the term “inner” refers to a portion of the intranasal device 300 disposed or enclosed by an outer surface, such as the interior compartment of a reservoir 380 (for example, the vial or reservoir storing the drug formulation), and the interior chamber of the actuator. Likewise, the term “outer” refers to a portion of a component disposed further from the interior or along the exterior surface of the intranasal device 300.

As shown in FIG. 3, for purposes of reference, intranasal device 300 can be seen to include a vial, bottle, or reservoir 380 to which is attached a pump assembly (“pump”) 382 (e.g., connected to a fluid opening of the reservoir 380). Collectively, for purposes of reference, the pump 382 and the reservoir 380 can comprise a main body or “intranasal device main body” or simply “main body” 330.

In different embodiments, an actuator assembly 350 is connected to the main body 330. An interior of the actuator assembly 350 is in fluid communication with the reservoir 380. In some embodiments, the actuator assembly 350 can include an actuator body (“actuator”) 310 and a flange component (“flange”) 320. The pump 382 can include a dispenser mechanism in fluid communication with the reservoir 380. Additional or alternate nasal spray device structural features can be provided using the proposed actuators and flanges, such as those known by persons skilled in the art.

In different embodiments, the intranasal device 300 includes two or more discharge orifices and related flow and delivery control components, such as internal conduits or channels, for depositing a quantity of formulation in form of a nasal spray in various areas of the nasal cavities, in particular to the olfactory cleft area and the VNO (for example, see FIGS. 1A and 1B). In some embodiments, this quantity may be metered. The intranasal device 300 thereby can include a dispenser system or other mechanism that-upon actuation--dispenses a metered dose of fluid through these opening(s) provided in the actuator body 310.

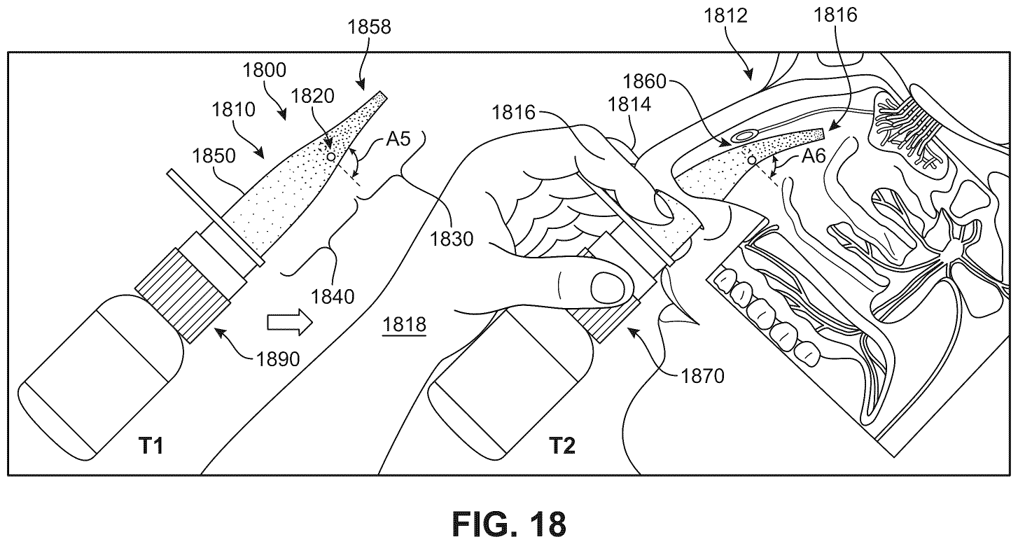

As depicted in FIG. 3, in different embodiments, the actuator body 310 can include a hollow tubular chamber of varying diameter bounded by a substantially continuous, semi-curved/semi-conical outer sidewall 308. The sidewall 308 can substantially enclose a fluid delivery assembly within. In different embodiments, the sidewall 308 that serves as the outermost surface of actuator 310 can comprise varying regions of flexibility and rigidity, as will be discussed with reference to FIG. 18.

In the embodiment shown in FIG. 3, the intranasal device 300 can include at least two delivery outlets on actuator 350: a first outlet 316 (that is, an opening or discharge orifice) for axially-oriented fluid streams or sprays, and a second outlet 360 (that is, an opening or discharge orifice) for laterally-oriented fluid streams or sprays, where each outlet can receive a metered portion of the dose as it is ejected into the actuator chamber and direct the spray to the external environment as it emerges from the respective outlet. In this case, the first outlet 316 is provided at a distal tip portion 352 (topmost region) of the actuator body 310 (facing generally upward or “North”), while the second outlet 360 is formed in a first lateral region (facing out to the side radially or “East/West”) of the actuator body 310.

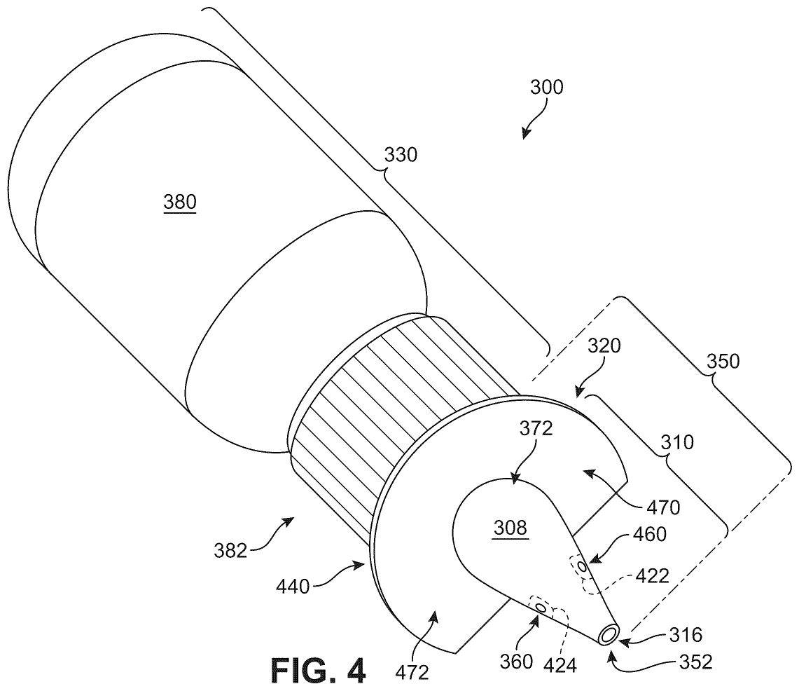

Thus, in one embodiment, the first outlet 316 is formed at an end of the distal tip portion 352 of the actuator body 310. In addition, in some embodiments, the second outlet 360 is formed as a through-hole channel passing or extending through a thickness of the actuator sidewall 308. In different embodiments, the intranasal device 300 can also include a third outlet (not shown in FIG. 3) that is formed on a side opposite to the side with the second outlet 360. The presence of the second outlet along with this optional third outlet (for example, see FIGS. 4, 7, and 10) can allow for the intranasal device 300 to be used for drug delivery to the VNO when inserted into either the left-side nasal cavity or the right-side nasal cavity, as noted above. In some embodiments, the second outlet 360 and the third outlet can be disposed symmetrically relative to the longitudinal axis 312.

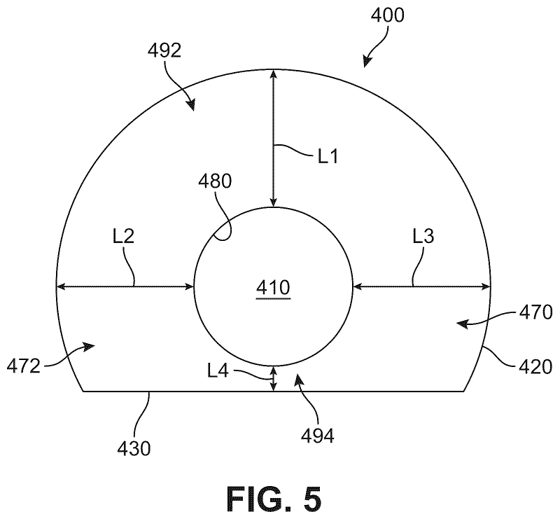

In different embodiments, the device 300 can include provisions for guiding insertion of the actuator 310 to facilitate the correct and desired alignment and orientation of each outlet relative to the chemosensory receptors. For example, as shown in FIG. 3, intranasal device 300 includes of a finger-grip actuator flange (or “flange”) 320 that is disposed around a base portion 372 of the actuator (that is, toward the bottom). In some embodiments, the flange 320 can include an opening or hole through which the actuator base snugly passes through, so that the flange 320 can be understood to surround the base portion 372 of actuator body 310 while resting on a surface of a pump 382 below. In other embodiments, the flange 320 can be integrally formed with the actuator body 310. In different embodiments, the flange 320 is shaped so that the actuators can only be inserted into the correct left and right nostrils, for example, via a bulge-shape on one side that ensures the actuator cannot be inserted into the nares if that would result in the lateral discharge orifices being oriented the wrong way (that is 180 degrees in rotation) to effectively target the vomeronasal pit and surrounding VNO area. The flange 320 will be discussed in greater detail below with reference to FIGS. 4 and 5.

In different embodiments, the intranasal device 300 can include reservoir 380 containing one, two, or more doses of fluid or other pharmaceutical dosage forms (for example, powder(s)) that can be dispensed in response to the actuation of the device. For many of the embodiments described herein, a person skilled in the art will understand that the drug to be delivered is formulated in a liquid or fluid that further contains various excipients. A skilled artisan will understand that excipients may be varied in order to adjust viscosity, adhesion, particle size and other parameters of nasal spray plumes. In some embodiments, the formulation comprises a liquid, gel, solid, powder, nanoparticles, vesicles or any combination thereof.

In some embodiments, the intranasal device 300 includes pump 382, which may be a pump, atomizer (including electronic atomizers), an aerosol valve, a piston sliding in the reservoir 380, or an air expeller, that is generally used to transfer a dose, on each actuation, towards an output orifice that-for example-is arranged at the axial end of an intranasal delivery device actuator as well as to one or more additional output orifices arranged along the lateral sides of the actuator. As will be understood by persons skilled in the art, certain embodiments will require duplicative reservoirs, dip tube/s, separate (or connected) pistons, and different internal geometries of the swirl chambers of the discharge orifice because of the different pressures, forces, and resulting spray patterns required for optimal targeting of the receptors in and around the vomeronasal pit and dorsal recess.

In other embodiments (not shown) the reservoir 380 may be connected separately by distinct conduits or channels that terminate for drug product release at each of the discharge orifices, for example as depicted in FIGS. 28-32. These pathways, together with the size and geometry of the discharge orifices, permit the predetermination of specified average droplet sizes, ejection pressure, and subsequent delivery pressure of the pharmaceutical product to its intended target area of chemosensory epithelium.

In this description, the portions of the intranasal device 300 that exclude the actuator assembly 350 (that is, the actuator body 310 and optionally the flange 320) will be referred to as the intranasal device main body 330. In some embodiments, the intranasal device main body 330 can include provisions and features of intranasal device devices known by those skilled in the art. In FIG. 3, the intranasal device main body 330 includes at least the reservoir 380 and the pump 382, which are fluidly connected and secured/coupled to one another. In some embodiments, the pump 382 is also in fluid communication with and coupled to the actuator body 310, thereby permitting the flow of the drug formulation from the reservoir to the actuator body 310. In some embodiments, the actuator body 310 and/or the flange 320 may be permanently or integrally attached or joined to the intranasal device main body 330. In other examples, the actuator body 310 and/or the flange 320 may be removably mounted or secured to the intranasal device main body 330.

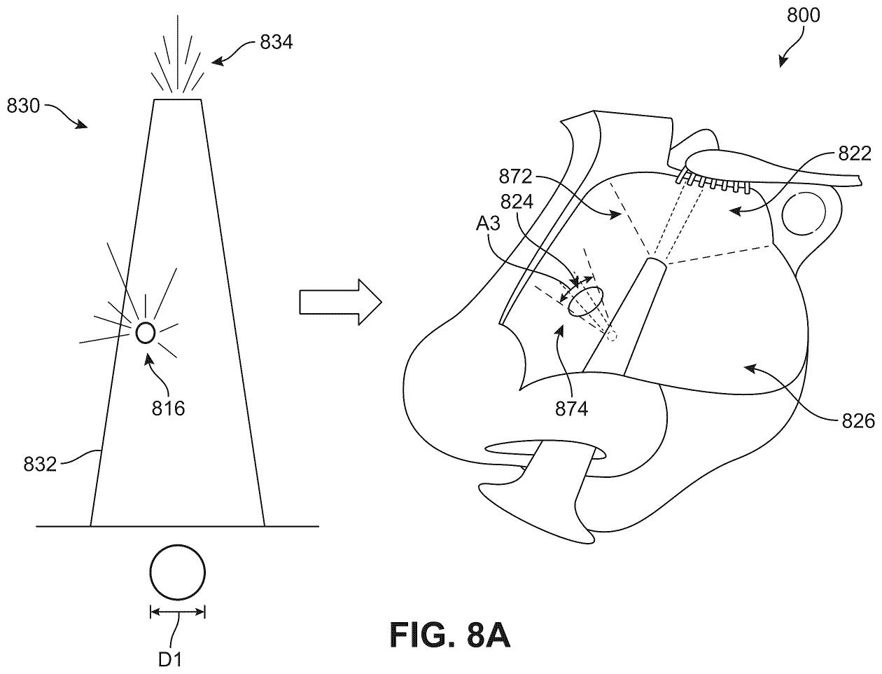

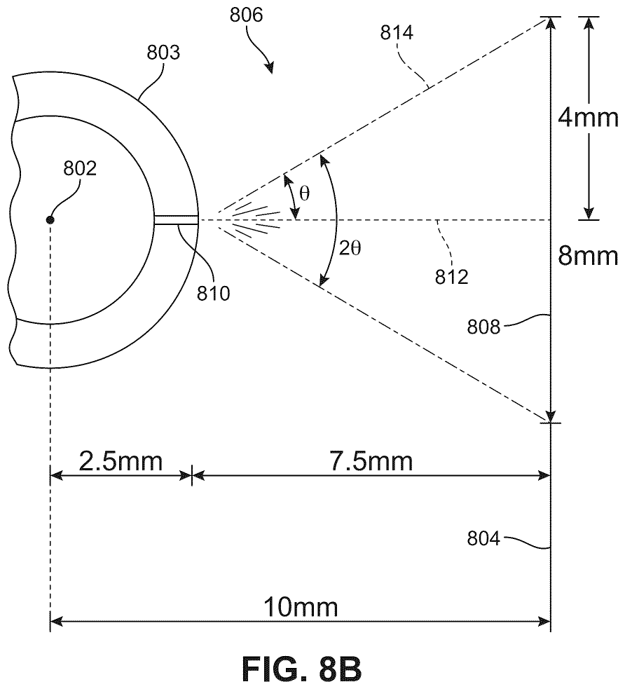

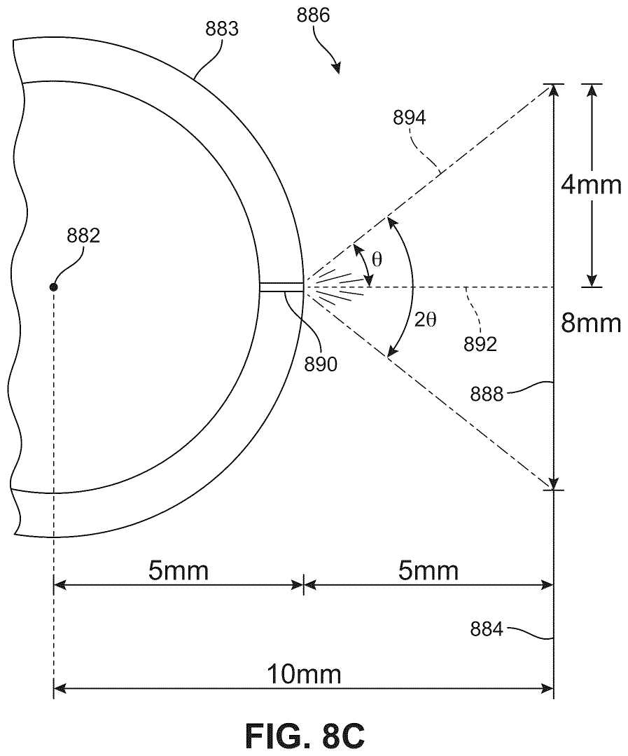

In different embodiments, the pump 382 in cooperation with actuator 350 may generate the output, for example, by generating an aerosol of a powder or a liquid in a flow of air or of gas. In different embodiments, upon actuation, a pre-measured dose of the drug stored in reservoir 380 can travel from the reservoir 380, through pump 382, and into the actuator body 310. Once actuation causes a dose to enter into an interior chamber of actuator body 310, the geometry, orientation, and location of each of first outlet 316 and second outlet 360 of actuator body 310 can define a drug dispersal pattern that can target both the primary chemosensory site associated with the olfactory region, and the selected secondary chemosensory site associated with the VNO. Thus, in different embodiments, the spray profile for intranasal device 300 can include a first spray plume that emerges from the first outlet 316, as well as a substantially simultaneous second spray plume that emerges downstream from the first outlet 316, exiting via the second outlet 360. Further details regarding the spray plume characteristics will be provided with reference to FIGS. 8A-8C below. It should be understood that the components and process described above will be duplicated in order to provide the separate discharge orifices and spray plumes.

In addition to the size, orifice geometry and configuration of the actuator as discussed above, additional elements of the device are relevant to achieving a favorable human factor design. In this regard, information regarding the flange 320 is now provided with respect to FIGS. 4, 5, 6, and 7. In FIG. 4, the intranasal device 300 is depicted in an orientation that reveals not only the axially-oriented first outlet 316 and the laterally-oriented second outlet 360, but also an (optional) additional laterally-oriented third outlet 460. In this example, the second outlet 360 is formed as a channel extending through/within a thickness of a first lateral region 424 of the sidewall 308 comprising the actuator body 310, and the third outlet 460 is formed as a channel extending through/within a thickness of a second lateral region 422 of the sidewall 308 comprising the actuator body 310.

The surface area and shape of flange 320 can be more clearly observed in both FIGS. 4 and 5. For example, in FIG. 4, the flange 320 is shown as disposed between the actuator body 310 and the pump 382, directly adjacent to and surrounding base portion 372 of the actuator body 310. The flange 320 can include a first finger grip portion 470 on one side of the actuator body 310, and a second finger grip portion 472 on the opposite side of the actuator body 310. Each of these portions is sized and dimensioned to comfortably receive the grip associated with one of an index finger, ring finger, or middle finger for purposes of actuation. The flange 320 can include a peripheral thickness 440 that is sufficient to ensure the flange 320 maintains its shape, that is, remains sufficiently flat or unbending when a force is applied by two human fingers in a downward direction.

In different embodiments, flange 320 can include provisions for ensuring the orientation of the actuator body 310 as it is inserted into a human nostril is correct and appropriate for the desired administration of the drug. For example, referring to FIG. 5, in some embodiments, the flange 320 has a symmetric shape relative to a sagittal-oriented midline that can serve to guide the user as they move the intranasal device 300 toward their face. In this isolated top-down view 400, the flange 320 can be seen to include a substantially continuous semi-circular shape in which an upper peripheral edge (“upper edge”) 420 is curved (for example, similar to a sunrise shape) and is joined to a lower peripheral edge (“lower edge”) 430 that comprises a flat line shape (for example, similar to a horizon for the sunrise-shape above).

In one example, the flange 320 has a half-circle shape. In another example, the flange 320 has a semi-oval or semi-elliptical shape. In another example, the flange 320 has an arch shape. In addition, in some embodiments, the flange 320 includes an opening 410 that is configured to surround and fixedly adhere to the outer surface of the base portion of the actuator body 310. For example, a thickness of an inwardly-facing edge 480 can be attached to the outwardly-facing surface of the actuator body 310. In other words, the flange 320 is positioned to wrap around and extend outward (radially) from the actuator's base. In other embodiments, the flange 320 can be integrally formed with the actuator's base as a substantially continuous material.

Dimensions associated with the flange 320 can facilitate user compliance with the intranasal device. For example, the flange 320 includes a first length L1 between the opening 410 and a topmost point of the upper edge 420, a second length L2 between the opening 410 and a leftmost point of the upper edge 420, and a third length L3 between the opening 410 and a rightmost point of the upper edge 420, where each of these three lengths are substantially similar or the same. However, a fourth length L4 extending between the lowest part of the opening 410 and the periphery of lower edge 430 is significantly smaller than any of L1, L2, and L3. In some embodiments, L4 is at most ¼ the size of L1, L2, or L3. The difference in lengths between the upper edge 420 and lower edge 430 from the opening 410 can help ensure that the device can only be fitted or inserted in one orientation relative to the human nose, thereby improving user comfort and compliance.

In other words, in different embodiments, the variation in the flange's dimensions around the base portion of actuator body 310 can serve as a tangible guide for the user so that, during application, the user must always orient the lower edge 430 on the side closest to the user's face, while the upper edge 420 is oriented away from the user's face. This is achieved by the relatively narrow “lip” portion that extends from the actuator body 310 to the lower edge 430, while the upper edge 420 extends much further away from the actuator body 310, thereby preventing the actuator assembly 350 from being insertable into a nostril from any orientation but the one where the lower edge 430 is brought into close proximity or contact with the user's face.

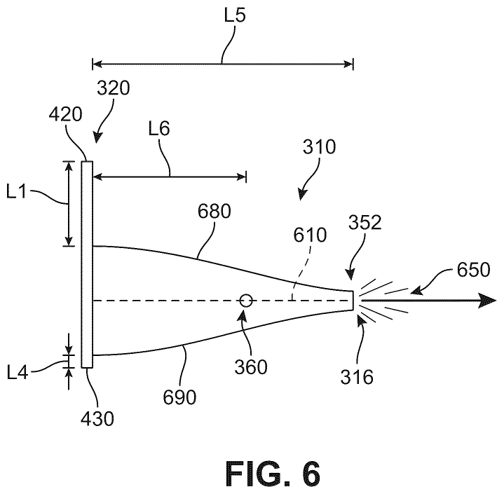

For purposes of clarity to the reader, FIG. 6 presents a side view of the actuator body 310 and flange 320 in isolation. In this drawing, the greater distance of length L1 of the flange 320 toward the “top” relative to smaller length L2 along the bottom can be better observed. Furthermore, FIG. 6 depicts the relative arrangement of the distal discharge orifice with the lateral discharge orifice(s). As noted earlier, in different embodiments, the dispenser mechanism for the proposed device may generate the output, for example, by generating an aerosol of a powder or a liquid in a flow of air or of gas. Actuation causes a dose to enter into an interior chamber of actuator body 310, and the geometry, orientation, and location of each of the outlets can define a drug dispersal pattern that can target both the primary chemosensory site associated with the olfactory region, and the selected secondary chemosensory site associated with the VNO. Thus, in different embodiments, the spray profile for intranasal device 300 can include a first spray plume 650 that emerges from the first outlet 316, as well as a substantially simultaneous second spray plume that emerges downstream from the first outlet 316, via the second outlet 360, in this case directly towards the viewer.

In order for each spray plume to accurately target its intended olfactory chemosensory epithelium sites, in different embodiments, the device includes an actuator with a distal discharge orifice (first outlet 316) having a length that brings the distal discharge orifice within about 1 to 3 cm, most preferably about 4 cm from the nostril in most adult humans. This distal discharge orifice can therefore administer the drug spray to the primary target area of chemosensory neurons (i.e., the olfactory cleft) without the need to also target the vomeronasal organ (which is targeted by the second outlet 360) and without as much loss of spray below the dorsal cleft as is the case with conventional actuators. As noted earlier, actuator dimensions and form factor considerations can reduce the need for patient training in the proper placement of an actuator in their nostrils while also improving the consistency of the administration of drugs to predetermined target areas among a diverse population.

This can be observed in FIG. 6, where the actuator has a fifth length L5 from flange 320 to first outlet 316 at distal tip 352, and a smaller sixth length L6 from flange 320 to second outlet 360. In this example, the actuator's length L5 can be substantially longer than a conventional length of about 2.5 cm, extending to a length in the range of about 3.5 to 4.5 cm, preferably around 4 cm, to release the upward spray plume closer to the dorsal recess near the olfactory cleft. Such proximity would not be otherwise possible using conventional actuators. As will be appreciated by persons skilled in the art, actuators of various lengths are also contemplated, including about 2.6 cm, 2.7 cm, 2.8 cm, 2.9 cm, 3.0 cm, 3.1 cm, 3.2 cm, 3.3 cm, 3.4 cm, 3.5 cm, 3.6 cm, 3.7 cm, 3.8 cm, 3.9 cm, 4.0 cm, 4.1 cm, 4.2 cm, 4.3 cm, 4.4 cm, 4.5 cm, 4.6 cm, 4.7 cm, 4.8 cm, 4.9 cm and 5.0 cm. These increased dimensions offer the advantage of relatively less dispersion and rebounding of spray from side to side of the nasal cavity, which reduces the loss of drug that would otherwise be wasted when administering pherines in coating the turbinates, which lack olfactory chemosensory neurons. With this longer actuator, as shown in FIG. 6, the lateral discharge orifices (for example, second outlet 360 and optionally a third outlet on the opposing side) would be placed about 1.5 to 2.5 cm from the flange 320, and preferably around 2 cm.

In other embodiments not shown here, the actuator can be about conventional length, that is, about 2.5 cm long, and there is a discharge orifice at the distal tip as described herein. However, in this alternate embodiment, the lateral discharge orifice(s) can be moved “up” in the distal direction such that they are directly below or closely adjacent to the distal tip 352. In other words, the lateral orifice would then be in a favorable location relative to the VNO to deliver a spray plume having appropriate dimensions and pressure values as described in various sections of this specification.

Furthermore, in different embodiments, while one lateral discharge orifice can reduce the waste of drug, it is more likely a user will be able to consistently insert the device in a way that correctly targets the desired region when there are two lateral discharge orifices along opposite sides of the distal tip of the actuator, even though one of the sprays would be wasted because the fluid would be directed in the opposite direction (away from medial septum of the nose and the vomeronasal organ). Thus, in some embodiments, it can be preferred to include two lateral discharge orifices on opposite sides of the actuator body sidewall, despite some portion of the drug being routed with less effectiveness out toward a lateral wall of the nose. In one example, the two lateral orifices are around 180 degrees apart from one another along the actuator sidewall so that a plane parallel to the sagittal plane extending between one lateral orifice to the other would cut the body of the actuator in two substantially symmetrical halves. In some embodiments, fluid flow can be managed and regulated so that when the device is inserted into a right-side nostril and the user engages the pump, drug travels out only via the distal opening and a first lateral opening on the left side of the device (septum-side), and when the device is inserted into the left-side nostril and the user engages the pump, drug travels out only via the distal opening and a second lateral opening on the right side of the device (which is now septum-side).

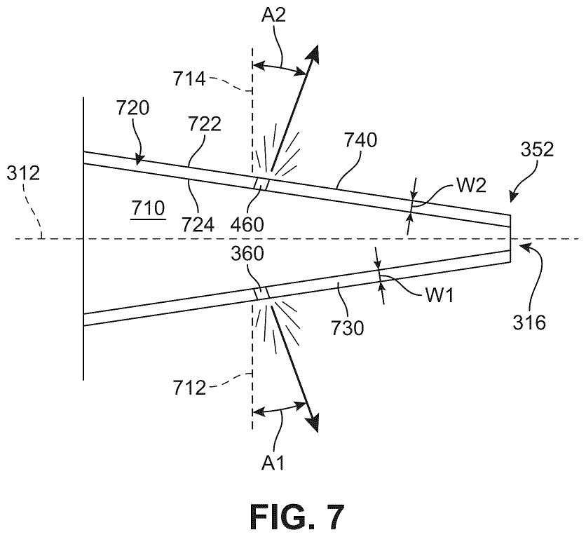

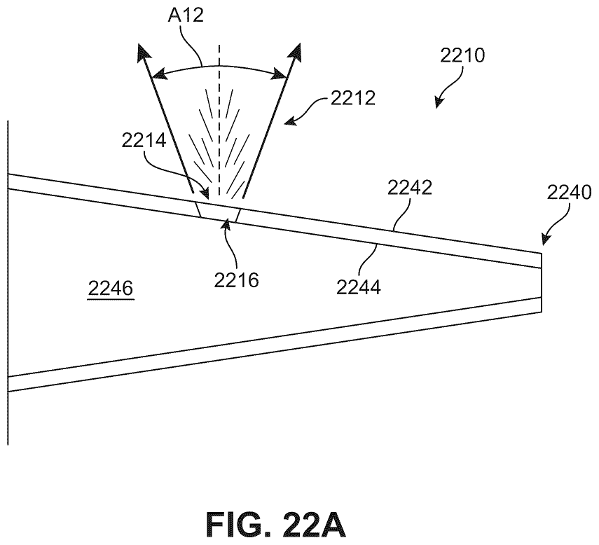

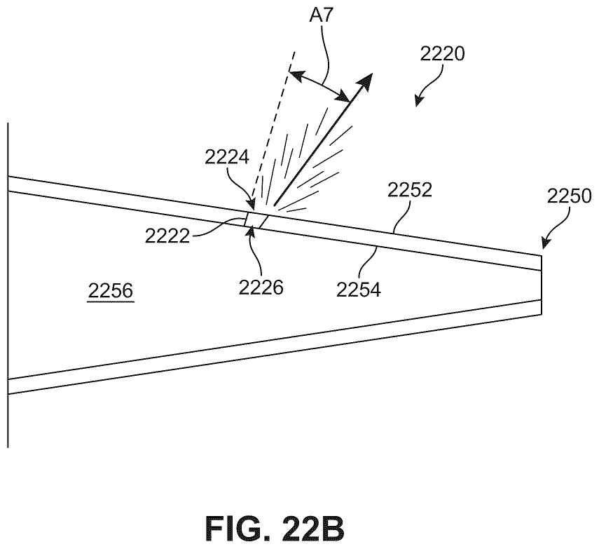

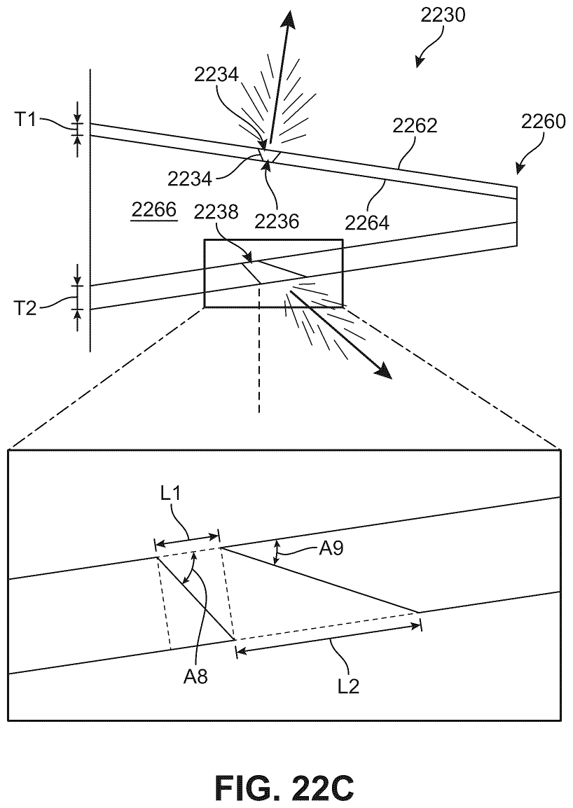

For purposes of clarity with respect to the spray plume orientation of the lateral discharge orifices, FIG. 7 depicts a top-down cross-sectional view of the actuator body 310 that more clearly reveals the discharge orifices for the drug provided via the laterally-facing second outlet 360 and the laterally-facing third outlet 460 that each generate separate sprays to the vomeronasal organ. In this cross-section, an interior chamber 710 of the actuator body 310 is depicted, bounded by a continuous actuator sidewall 720 that serves as the actuator housing or enclosure. The actuator sidewall 720 includes an exterior surface 722 and an opposite-facing interior surface 724, separated by an actuator thickness. In different embodiments, the thickness can be substantially uniform throughout the sidewall 720. For example, a first width W1 on one part of the sidewall 720 (for example, along a first sidewall portion 730 which includes the laterally-oriented second outlet 360) and a second width W2 along a different part of the sidewall 720 (for example, along a second sidewall portion 740 which includes the third outlet 460) can be approximately equal. In this case, the spray angles A1 and A2 are also substantially similar due to the orientation of each of the channels. However, it should be appreciated that in other embodiments (for example, see FIG. 22C), the thickness can vary, particularly with respect to the sections of the sidewall 720 through which an outlet is formed. In other words, in some embodiments, first width W1 and second width W2 can differ from one another, such that one is thicker or wider than the other. This variation can allow for important differences in the depth of the channel that can be formed for each outlet, which can be used to influence the velocity and distance the plume can achieve before beginning to dissipate or disperse.

### VI. Discharge Orifice Considerations for Delivery Optimization

It can be appreciated that, as a general matter, the characterization of spray pattern and plume geometry is important for evaluating, controlling, and managing the performance of an intranasal device and its drug delivery efficacy. For example, various factors can affect the spray pattern and plume geometry, including the size and shape of the actuator body, the design of the pump, the size of the metering chamber, and the characteristics of the formulation. As will be known to persons skilled in the art, parameters that influence and determine the properties of the plume and subsequently the deposition pattern of the particles include the swirl effect, nozzle orifice dimensions, the spray cone angle, and the break-up length. See for example, https://www.proveris.com/wp-content/uploads/2019/09/Spray-Pattern-as-a-Screening-Tool-for-Nasal-Sprays-article.pdf, and https://aptar.com/wp-content/uploads/2022/04/Overview-of-Intranasal-drug-administration-using- multi-dose-nasal-spray-pumps.pdf both of which are incorporated herein by reference in their entirety.

Beyond these factors, the characteristics of the outlet discharge orifices/channels and their relative positions along the actuator body can also affect spray pattern and plume geometry by impacting the fluid dynamics of the drug particles as they exit. Typically, the plume evaluation includes plume angle, width and height, and the spray pattern is evaluated for maximum diameter (Dₘₐₓ) and minimum diameter (Dₘᵢₙ). The desirability of a particular value in these measurements depends on the region where drug delivery is desired.

As noted earlier, the area of the nasal cavity, including the nasal valve, has a particular shape. For adults, on average, the valve extends over about 1 centimeter (cm) in depth, has a vertical longitudinal section of about 3 cm to 4 cm, and a width of about 1 millimeter (mm) to 3 mm. Beyond the nasal valve, the nasal cavity includes a larger cavity (about 7 cm in height by 2 cm to 3 cm in width). The conchae face the nasal valve. The roof of the nasal cavity is situated above the conchae, which roof includes the ethmoid sinuses, the olfactory bulb, and the olfactory nerve. Generally, for nasally delivered substances, the deposition site may also influence the extent and route of absorption along with the target organ distribution.

In other aspects of the embodiments not relevant to pherine administration, the nasal spray device may be used for treatments in which absorption of the drug product is intended. For such purposes, the active ingredients that are absorbed from the anterior regions of the nasal cavity are more likely to drain via the jugular veins, whereas drugs absorbed from the mucosa beyond the nasal valve are more likely to drain via veins that travel to the sinus cavernous, where the venous blood comes in direct contact with the walls of the carotid artery. A substance absorbed from the nasal cavity to these veins/venous sinuses will be outside the blood-brain barrier (BBB), but for substances such as midazolam, which easily bypass the BBB, this route of local “counter-current transfer” from venous blood may provide a faster and more direct route to the brain. Some studies have suggested that there may be a preferential, first-pass distribution to the brain through this mechanism after nasal administration for some, but not all small molecules.

It should be appreciated that, even if pherines did penetrate the BBB, there would be no response (that is, nothing would happen physiologically as they are specific agonists to NCN receptors, and would not bind to any receptors in the brain. Extensive in vitro binding assays have been performed showing lack of binding to steroid, hormone, and neurotransmitter receptors across multiple candidate pherines in our platform (for example, fasedienol, itruvone, and PH80. Moreover, pherines are metabolized locally in the nasal mucosa by CYP 450 enzymes in the nasal epithelia, and completely flushed out of the nasal passages approximately every 20 minutes as the mucosa turns over. Finally, the low spray velocity minimizes the likelihood of penetrating the tight junctions in the nasal epithelia that protect the brain from foreign substances. In some cases, formulations that prolong the time the active drug stays in the nasal mucosa may also reduce the likelihood of BBB penetration.

When selecting an actuator, the challenges imposed by the dimensions of the nasal structures and target areas, as well as the sensitivity of the mucosa in the vestibule and in the valve area, can be critical to how the routing of the drug to the systemic circulation or CNS target sites might occur. In addition, in some cases, direct contact of the distal portion (including the tip) of the actuator with the nasal epithelium during actuation may be preferable, depending on the nature of the product to be delivered. Persons skilled in the art also will recognize that physical contact and the nature of the drug product and its delivery vehicle may create mechanical irritation and injury to the mucosa resulting in nosebleeds and crusting, and potentially erosions or perforation, making direct physical contact between the actuator against the septum wall and the VNO less desirable.

FIGS. 8A-8C provide further description regarding the plume characteristics and dispersal cone angle that can be implemented by the proposed devices for accurately targeting multiple and discrete chemosensory sites in the nose. In FIG. 8A, a first actuator 830 is presented in an isolated side view. The first actuator 830 includes both a first outlet 834 and a second outlet 816. The second outlet 816 includes a through-hole discharge orifice formed in a sidewall 832 of the first actuator 830. In this example, the discharge orifice comprising the second outlet 816 has a substantially circular or round shape. When the first actuator 830 (as part of an intranasal device, not shown here) is inserted by a user 800 into a nasal cavity 826 and actuated, a first plume 872 is ejected axially from the opening at the distal tip, targeting a first region 822 (in this case including the olfactory region/cleft). In addition, a second plume 874 is ejected laterally from the circular discharge orifice on the sidewall corresponding to the second outlet 816, targeting a second region 824 (in this case, targeting the VNO), with a spray angle A3. The position of the second outlet 816 relative to the VNO allows the second plume 874 to deliver a dose of the drug to the VNO with rapid overall clearance while a spray plume geometry angle of A1 permits the spraying of a broad area in a superior and inferior direction around the VNO.

Moving now to FIGS. 8B and 8C, additional details regarding the relationship of the distribution cone angle with the size of the discharge orifice and actuator are provided. It can be appreciated that in many cases, a larger distribution cone angle would be preferable, particularly in light of the variability in the exact site of the VNO from person to person. It should be appreciated that the lateral discharge orifices are positioned relative to the flange so that the spray going in the direction of the vomeronasal pit is optimized to cover it, notwithstanding variability in nasal anatomy. In other words, the spray may need to be “wider” at the point of release than the spray from the distal tip of the actuator or it may need to expand much more rapidly and widely after release before touching the vomeronasal pit.

In FIG. 8B, a schematic drawing is shown. For purposes of this example, after insertion of an actuator 803, a centerline 802 of actuator 803 can be approximately 10 mm from a medial wall 804 (also referred to as the septum of a nasal cavity 806). Due to the variability in human anatomy discussed above, the VNO may be located anywhere within a target region 808 (that is, target region 808 includes an area that most likely encompasses the VNO in most humans). Target region 808 generally can be located diagonally upward about 10 to 30 mm along septum 804 relative to the nostril.

In the embodiment shown in FIG. 8B, target region 808 extends in the anterior-posterior direction along septum 804. As would be appreciated by a person skilled in the art, the location of the VNO may also vary in the superior-inferior direction as well. Because of these variations, a full cone spray pattern may be used to distribute the compound throughout target region 808. In this way, the chance of distributing the compound to the VNO is maximized.

Thus, some embodiments can include provisions to distribute the compound towards target region 808. As described herein, one or more lateral orifices 810 may be provided in actuator 803. Lateral orifices 810 may be configured to distribute the compound throughout target region 808. The location, size, direction and spray pattern of lateral orifices 810 may be modified to enhance the ability of each of the lateral orifices 810 to distribute the compound throughout target region 808.

For purposes of illustration, two examples are provided herein describing a method to calculate the spray angle of lateral orifice 810. Using the principles from these two examples, any given lateral orifice on any sized actuator may be configured to distribute the compound over any desired target region.

In a first example shown in FIG. 8B, the actuator 803 diameter is about 5 mm, with a radius of about 2.5 mm. The center of actuator 803 is about 10 mm from septum 804. Based on these values, the distance from the sidewall of actuator 803 to septum 804 can be estimated to be about 7.5 mm (that is, corresponding to the difference between the 2.5 mm radius and the overall distance of 10 mm). Assuming in this example that target region 808 extends about 8 mm along septum 804, this provides a 4 mm region from the centerline 812 of spray 814.

Defining a spray angle of 2θ, half of the spray angle can be defined as θ. Using trigonometric relationships known to those skilled in the art, tan θequals 4/7.5. From this we can determine θ equals tan-1 (4/7.5), and θ equals 28.1°. Based on these values, 2θ equals 56.2°. Therefore, a 56.2° spray pattern is needed from lateral orifice 810 disposed on a 5 mm actuator 803 to distribute the compound over an 8 mm target region that is located 10 mm away from the center 802 of actuator 803. Repeating this calculation for target region 808 that extends 10 mm along septum 804 yields a spray angle of 67.4°.

In a second example shown in FIG. 8C, an actuator 883 diameter is 10 mm, with a radius of 5 mm. A center of actuator 883 is about 10 mm from septum 884. Based on these values, the distance from the sidewall of actuator 883 to septum 884 can be estimated to be about 5 mm (that is, corresponding to the difference between the 5 mm radius and the overall distance of 10 mm). Assuming in this example that target region 888 extends about 8 mm along septum 884, this provides a 4 mm region from the centerline 892 of spray 894.

Defining a spray angle of 2θ, half of the spray angle can be defined as θ. Using known trigonometric relationships known to those skilled in the art, tan θ equals 4/5. From this we can determine θ equals tan-1 (4/5), and θ equals 38.7°. Based on these values, 2θ equals 77.4°. Therefore, a 77.4° spray pattern is needed from lateral orifice 890 disposed on a 10 mm actuator 883 to distribute the compound over an 8 mm target region that is located 10 mm away from the center 882 of actuator 883. Repeating this calculation for target region 888 that extends 10 mm along septum 884 yields a spray angle of 90°. Thus, the proposed embodiments can thereby incorporate distance-to and size considerations of a desired target region by adjusting actuator dimensions as described herein. Additional context based on conventional actuator dimensions for the reader's reference are also provided Kapadia et al., “Comparison of a short actuator and along actuator spray in sinonasal drug delivery: a cadaveric study,” Ear, Nose & Throat 98(7): E97-E103 (2019).

### VII. Variations in Discharge Orifices Arrangement and Pattern

In different embodiments, given the variability in nostril size among people, in different embodiments, the actuator can preferably include multiple (for example, two, three, four, five, six, or more) of such sidewall discharge orifices. Other appropriate configurations will be readily available to persons skilled in the art. In some embodiments, these discharge orifices can be spaced over a length of about 1 to 3 cm in order for the discharged nasal spray to cover the vomeronasal organ as found in most people. Thus, in some cases, the spray dispersal pattern can also be modulated by the inclusion of multiple discharge orifices or discharge orifice sets along the sidewall of the actuator. One implementation of multiple discharge orifices has already been introduced, where two laterally-facing outlets are provided on the sidewall-each outlet being formed in generally opposing sides of the sidewall to facilitate application of the drug to the VNO in each nostril (that is, where the VNO's location relative to the actuator changes between the left nostril and the right nostril as the actuator is inserted in first one, and then the other, nostril). However, in other embodiments, there may be multiple discharge orifices can correspond to channels provided along the actuator sidewall so that each discharge orifice disposed in a location one atop the other (“stacked”). In one example, this can provide outlets where the individual channels are substantially aligned with or parallel to one another.

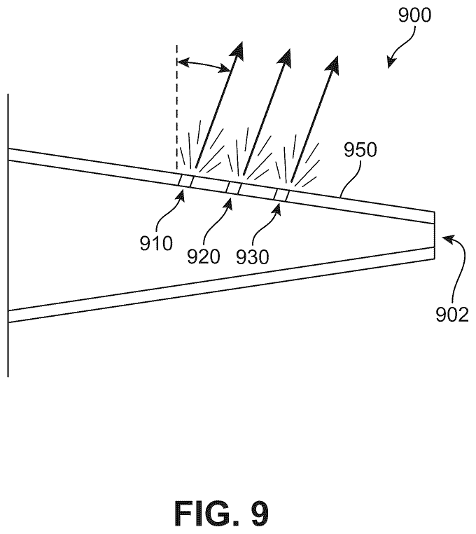

A non-limiting example of this arrangement is shown in FIG. 9. In FIG. 9, a top-down cross-sectional view of a second actuator 900 reveals the presence of a sequence of three laterally-oriented passageways for drug delivery comprising a first outlet 910, a second outlet 920, and a third outlet 930, along with an optional opening 902 formed at the distal tip for axial jet formation. Collectively, these three orifices can be referred to as a dispersal unit, in that they may be used to service one particular target region in the nasal cavity. In some embodiments, the use of multiple outlets can allow the drug delivery to maintain a focused plume from each orifice while also enabling the release of the drug across a broader area. In different embodiments, the sidewall 950 can include a plurality of pinhole discharge orifices, or sprinkle or scatter of orifices, that can serve to finely direct the release and delivery of drugs into a desired target area of the nasal cavity. The more the number of orifices that are stacked in such a manner, the wider the dispersal that can be provided.

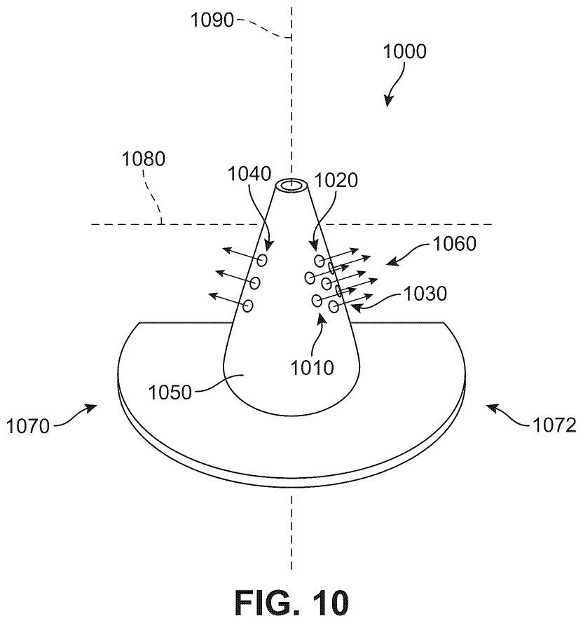

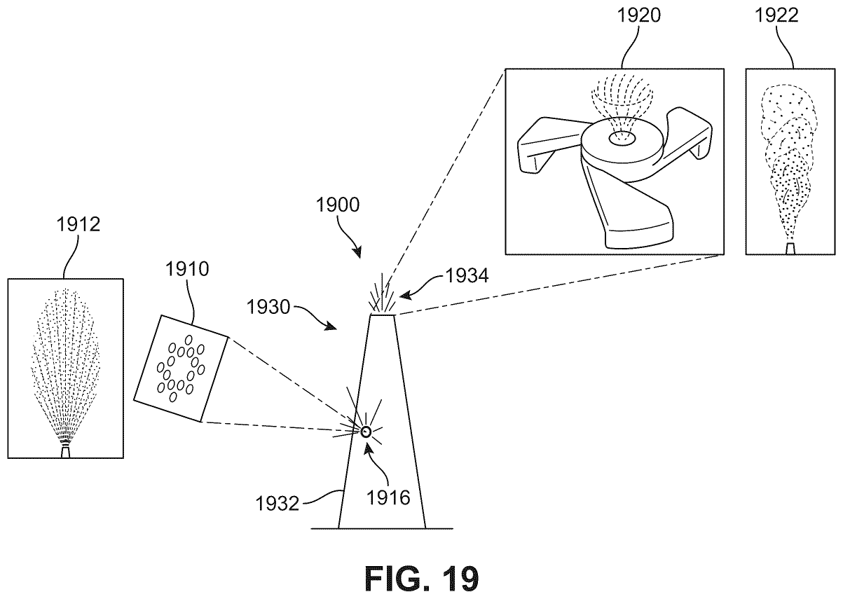

Referring now to FIG. 10, in still other embodiments, there may be multiple orifices formed not only above (distal) and/or below (proximal) one another (for example, with respect to a longitudinal axis 1090) but also side by side with respect to a lateral axis 1080. This approach is similar to the arrangement of two outlets to service both nostrils, but differs in that the additional orifices that are provided are distributed in a micro-pattern (dispersal units) so that each orifice is closely situated/closely proximate to another orifice with respect to either or both axes. These micro-patterns can be symmetric or asymmetric. For example, asymmetric patterns may be useful when drug delivery is needed in only one nostril, and different volumes of the drug need to be routed along different directions in one nostril. Additional details and alternatives related to such arrangements are described below in FIGS. 19-21E.

In FIG. 10, an isometric view of a third actuator 1000 depicts an example of this approach, where an actuator sidewall 1050 can include multiple “sets” of laterally-oriented discharge orifices. For example, along a first side 1070 of the sidewall 1050, a first set 1046 of orifices (including a first discharge orifice, a second discharge orifice, and a third discharge orifice) are arranged in a first portion of the actuator to provide a substantially straight “line” or column of drug delivery outlets, similar to the set illustrated in FIG. 9. However, in this case, the ninth actuator 1000 further includes a second set 1060 of orifices that are disposed along a second side 1072 of the sidewall 1050. In the second set 1060, there are three “lines” or columns of discharge orifices, including a first column 1010 (comprising two discharge orifices), a second column 1020 (comprising three discharge orifices), and a third column 1030 (comprising two discharge orifices). Collectively, this group of discharge orifices can be used to deliver a customized spray pattern to a pre-designated target zone within the nasal cavity. In addition, it should be understood that although the discharge orifices are shown as having substantially similar sizes and shapes in FIG. 10, in different embodiments, two or more discharge orifices may have a shape and/or size that differs from the other, as discussed earlier.

### VIII. Impact Pressure and Force Considerations

In different embodiments, depending in part on the dispenser mechanism selected, there may be variation in the parameters of the pharmaceutical expelled through each of the outlets, such as the speed of the particles and their density. These parameters can be adapted routinely to accommodate the condition being treated. For purpose of the present disclosure, preferred parameters are those that deliver the pharmaceutical compound to its intended target areas and do so with an impact pressure that avoids or minimizes activation of trigeminal mechanoreceptors, particularly the high-threshold trigeminal receptors, as discussed above.

It should be appreciated that both the primary outlet (for example, the distal axially-oriented orifice) and the secondary outlets (for example, the laterally-facing orifices formed in the thickness of the sidewall) are passive outlets configured to allow for consistency in the pressure that is experienced by the user during actuation. In other words, the speed of secondary flows of fluid exiting the lateral openings can generally match the speed of the primary flow of fluid that exits the distal opening, as both provide passive flow paths. However, it can be understood that with the variations in the structural characteristics of each opening as described herein, the resultant speed of the flow can be affected.

In order to avoid the production of secondary flows that are too strong, which could be uncomfortable for users and/or disadvantageously affect high threshold trigeminal mechanoreceptors, the speed of the compound as it exits the secondary openings can be modulated by the size and/or depth of each orifice, as well as the distance between the actuator and the target region, as illustrated earlier. Thus, orifices that re-direct the flow in an anterior (proximal) direction would cause a slowdown in the velocity of the flow, while orifices that permit the flow to continue to move in a generally posterior (distal) direction would allow for a relative increase in speed.

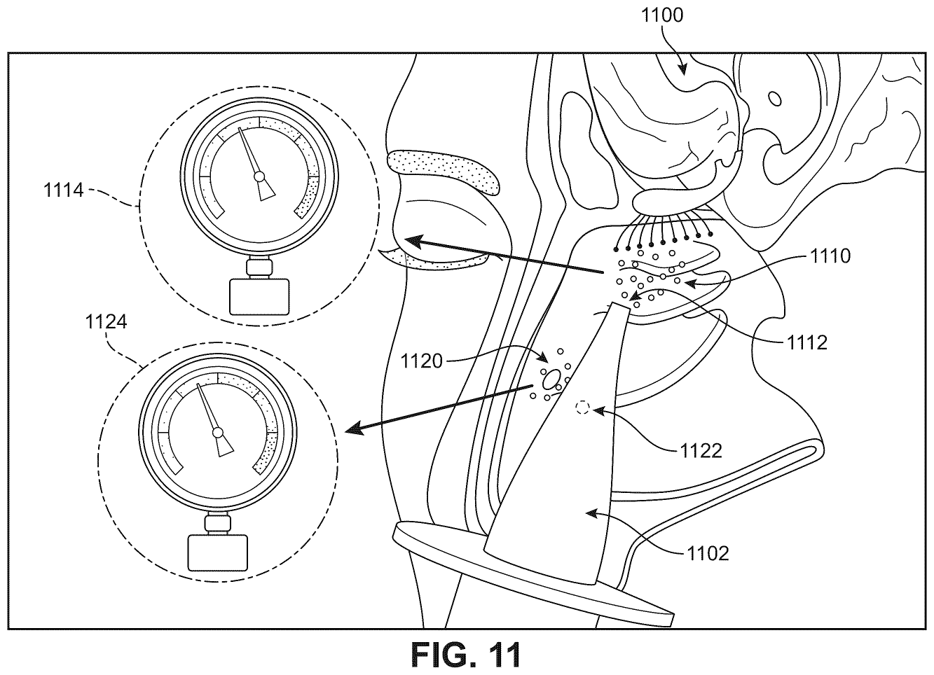

Referring to FIG. 11, it can be understood that in different embodiments, the proposed systems can be configured to balance the load of drug that is delivered through each discharge orifice, and thereby regulate the pressure (impact pressure) that would be experienced by a user. For example, with respect to actuator 1102 being actuated by a user 1100 in FIG. 11, a first spray 1110 is depicted exiting a first outlet 1112 toward a first region (for example, the olfactory region) in a generally upward or superior direction, while a different, second spray 1120 exits a second outlet 1122 toward a second region (for example, the secondary chemosensory site with the VNO) in a generally left-wise direction.

The pressure associated with each of these flows can be managed via custom modulation of each of the shape, size, orientation, and length of the discharge orifices. Thus, as shown in FIG. 11, the first spray 1110 (generated by the distal opening) is associated with a first pressure 1114 and the second spray 1120 (generated by the lateral opening) is associated with a second pressure 1124, where the two pressures are equivalent. In addition, these pressure levels are below the range that might potentially trigger an undesirable experience by the user 1100, for example as might result from the activation of trigeminal pressure receptors.

Thus, as described herein, in different embodiments, the arrangement of any two or more outlets in the actuator can be associated with substantially similar impact pressures. More specifically, the impact pressure of the release of the pharmaceutical substance on the chemosensory epithelium in the vicinity of the nasal cleft via the axially-oriented outlet can be substantially the same as the impact pressure of the release of the pharmaceutical substance on the chemosensory epithelium in the vicinity of the vomeronasal organ when taking into account the variation in distance between each discrete chemosensory target region and the orifice that is directing drugs toward that region. Furthermore, during operation of the intranasal drug delivery device, the actuation mechanism and actuator's physical characteristics can be configured such that the maximum impact pressure that will be exerted is less than the threshold pressure that otherwise would activate a substantial (for example, approximately 20-30%) percentage of the nasal trigeminal mechanoreceptors, thereby significantly reducing any likelihood that the plumes would cause discomfort to a user. In some embodiments, the impact pressure associated with the plume that is ejected from each outlet in an actuator for the intranasal drug delivery device is equal to or less than about 0.8-3.0 kPa.

Furthermore, in different embodiments, upon actuation of the intranasal drug delivery device, a first volume of the drug is released by the actuator, and approximately 10-30% of the first volume is applied to the chemosensory epithelium associated with the VNO. In another example, the laterally-oriented outlet(s) can be modified such that when a first volume of the drug is released by the actuator (upon actuation), only approximately 15-25% of the first volume is applied to the chemosensory epithelium associated with the VNO. In still another example, the laterally-oriented outlet(s) can be further modified such that when a first volume of the drug is released by the actuator (upon actuation), only approximately 20% of the first volume is applied to the chemosensory epithelium associated with the VNO. In some embodiments, the spray device incorporates provisions for pressure controls from all discharge orifices to reduce the likelihood of inadvertent activation of trigeminal mechanoreceptors.

In one preferred embodiment, the lateral discharge orifices are especially controlled since the spray travel distance is quite short, such that the lateral orifices produce a type of “gentle mist” while the distal end produces more of a spray effect. With this approach, the pressure associated with the lateral spray plume is less than that of the distal spray plume, so the resultant impact pressures at each target region are approximately and substantially about the same. In this way, the proposed spray device can ensure that the maximum pressures experienced by the nasal mucosa is less than the activation threshold of trigeminal mechanoreceptors or at least below the activation threshold of a substantial portion of trigeminal mechanoreceptors, particularly the high threshold mechanoreceptors. As described herein, the delivered pressure should preferably be less than the pressure that would activate the trigeminal nerve in a manner that interferes with the brain's processing of the olfactory nerve signal responsive to the administered pherine.

### IX. Dual actuator Embodiments

While the above discussion contemplated structural variations in discharge orifices, in order to improve the likelihood of a positive user experience and compliance, some embodiments can further include provisions for simultaneous bilateral delivery of drug. More specifically, in some embodiments, the intranasal spray device can include a double actuator structure. One non-limiting example of such a double actuator spray device 1200 is depicted in FIG. 12. In this case, double actuator spray 1200 includes standard spray device components as described herein, including but not limited to a reservoir 1280 and a dispenser mechanism 1282. In addition, a flange component (“flange”) 1220 is provided that can be substantially similar to flange 320 discussed earlier. However, it should be appreciated that in this embodiment, the dimensions of the flange 1220 may be adjusted in order to accommodate both a first actuator 1208 and a second actuator 1218 that are formed atop and extend distally upward from the flange 1280 in a direction aligned with a vertical axis 1280. In contrast to the single actuator spray device embodiments described earlier that entail the user to perform a sequence of actions to apply the drug to both nostrils (for example, insert in one nostril and actuate/spray, then flip device orientation and insert in adjacent nostril and actuate/spray), the double actuator spray device 1200 enables a single operation to deliver drug to both nostrils simultaneously. Each of the two actuators are inserted into a respective nostril at the same time, and a single actuation causes drug to be delivered into both nostrils, concurrently. In addition, the first actuator 1208 includes a first lateral discharge orifice 1260 that is facing a medial direction, and the second actuator 1218 includes a second lateral discharge orifice 1210 that is also facing the medial direction, so that the VNO in the left nostril and the VNO in the right nostril are both targets of the drug. In other words, in one example, the first lateral discharge orifice 1260 and second lateral discharge orifice 1210 are facing one another, and are aligned along an axis 1202 that is substantially parallel to a lateral axis 1290. Finally, the standard distal discharge orifice is also provided at the distal tip of the first actuator 1208 and the distal tip of the second actuator 1218, likewise enabling simultaneous drug delivery to the dorsal recess target region in both nostrils.

Such an arrangement can ensure that both nostrils receive actuators at the same depth, and also eliminates variability inherent in a single actuator placement. For example, by maintaining a uniform, consistent depth and angle to both nasal cavities and over multiple uses, as well as ensuring a consistent insertion angle further ensures both the olfactory cleft/dorsal recess and VNO receive the appropriate dose/volume (that is, the uniform dosing in two nostrils simultaneously). In addition, patient compliance can be improved, as it is far easier for a patient to take one spray for delivery to both nostrils simultaneously than administering the spray in two separate events.

### X. Further Actuator Embodiments for Improved Targeted Delivery to the Vomeronasal and Olfactory Cleft Regions

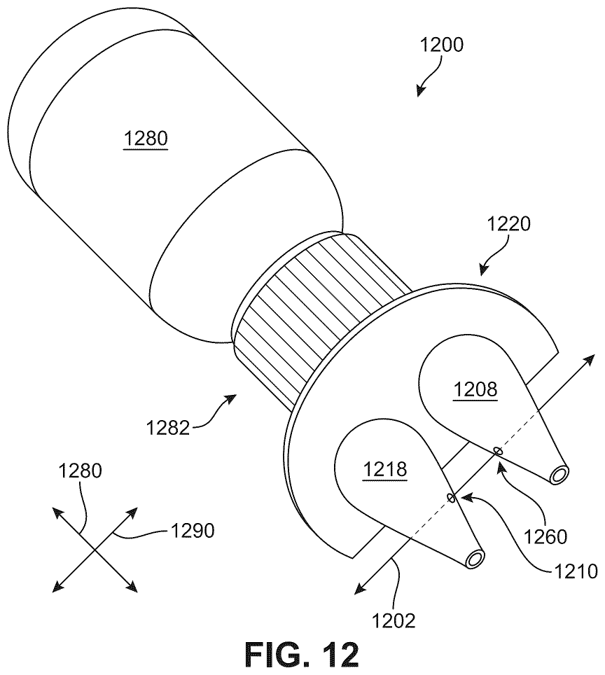

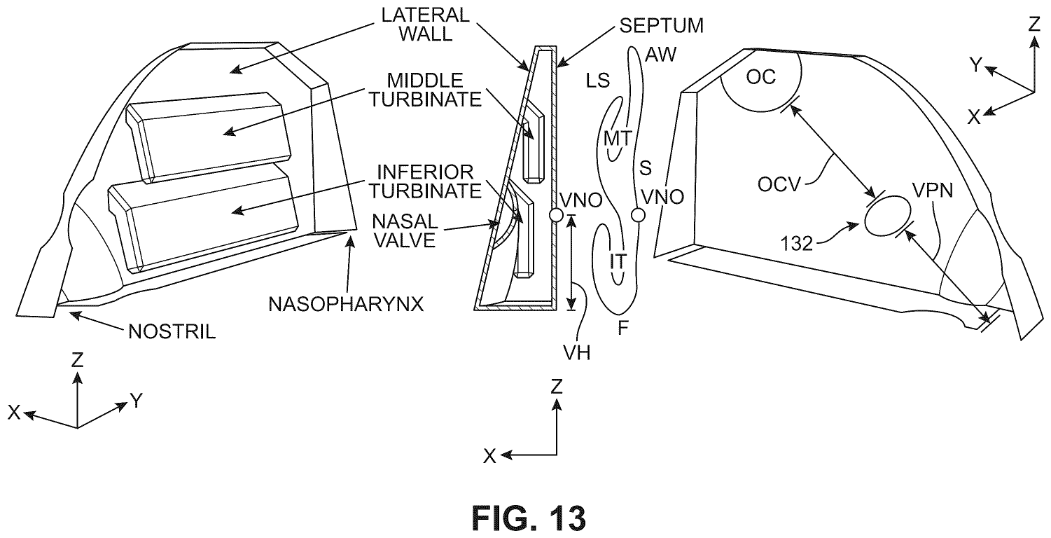

This section offers additional details directed to embodiments in which the device can be further tailored or modified to improve targeting of the desired delivery sites (for example, the VNO and the olfactory cleft). For purposes of context, FIG. 13 depicts an exploded view of a model of a leftward nasal cavity for a human. Toward the center of the drawing, a schematic cutaway view (along the coronal plane) of the nasal cavity is provided, revealing the nasal valve, inferior turbinate, and middle turbinate, bounded on each side by the lateral wall and septum (medial wall). Adjacent to the cutaway view is a schematic view of the flow passage through the nasal cavity (AW: anterior wall, S: septum, F: floor, LI: lateral wall inferior part, IT: inferior turbinate, LML lateral wall middle part, MT: middle turbinate, and LS: lateral wall superior part). For the reader's reference, a region of the septum where the vomeronasal pit (including the VNO) region is identified, at a vertical height of VH above from the level of the nostril opening. In humans, height VH typically ranges between 1 cm and 2 cm. In different embodiments, the vomeronasal pit region can be located on the septal wall near to and/or across from an upper half of the inferior turbinate, and in some cases near to and/or across from a lower end of the middle turbinate. In one example, the vomeronasal pit region is situated immediately above the nasal valve region.

A view of the lateral wall is opened toward the left side of FIG. 13, revealing more of the middle turbinate and inferior turbinate. Similarly, a view of the medial wall is opened toward the right side of the drawing, revealing the septum surface. For the reader's reference, a region of the septum where the vomeronasal pit (including the VNO) region is again identified in this view, as well as that of the olfactory cleft (OC). Axis “X” corresponds to a lateral axis aligned with a medial-lateral direction, axis “Y” corresponds to a sagittal axis aligned with an anterior-posterior direction, and axis “Z” corresponds to a vertical axis running in a superior-inferior direction. In this perspective, it can be better appreciated that the location of the OC is not only above (superior to) the site of the vomeronasal pit but relatively posterior as well. Thus, a distance OCV between the two regions extends in a substantially diagonal orientation. In humans, the distance OCV can range between 1.8 cm and 2.5 cm, and usually 2.1 cm. Similarly, the site of the vomeronasal pit region is not only above (superior to) the nostril, but relatively posterior as well, and a distance VPN between the nostril and vomeronasal pit region extends “back” and slightly “up” in a substantially diagonal orientation. In humans, the distance VPN can range between 1.8 cm and 2.5 cm, and usually 1 cm. With this context, the proposed arrangement and locations of the lateral discharge orifices in the embodiments described herein can be better understood. In addition, the presence of various protruding structures in the nasal cavity greatly influences the route a device must travel to reach different zones for drug delivery.

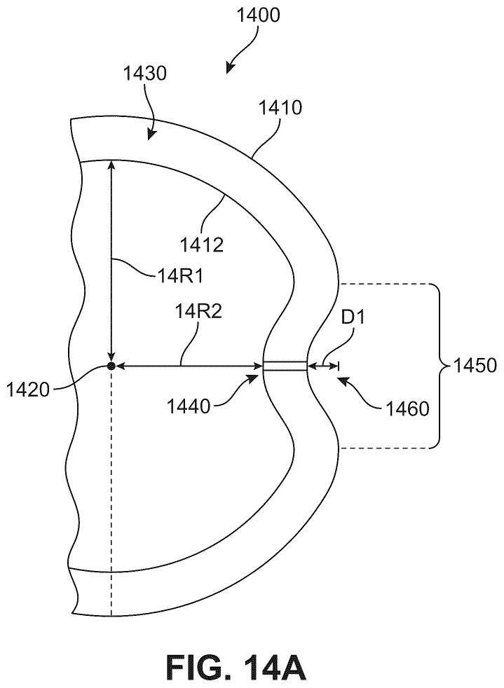

In different embodiments, the proposed device can include provisions for improving positioning of the actuator inside a human nose. For example, referring to FIGS. 14A and 14B, an alternate actuator embodiment whereby the lateral orifice is formed in a recessed portion of the sidewall is presented. In FIG. 14A, a schematic cross-sectional view of a portion of a fourth actuator 1400 is shown. In this view, the cross-sectional shape of the actuator can be observed to be substantially round, as described earlier, but further includes a “dip”, indentation, or recessed portion 1450 that slopes inward toward a central point 1420. Thus, a lateral orifice 1440 is now at the nadir of a curved or sloped region formed in the actuator sidewall. In other words, while a first radial axis length 14R1 extending from central point 1420 within the chamber to an inner surface 1412 of a sidewall 1430 is generally consistent around the chamber, when considering the distance from the central point 1420 to points along the recessed portion 1450, the distances are shorter. For example, a second radial axis length 14R2 extending from the central point 1420 to the deepest or lowest part of the recessed portion 1450 where an opening leading into the lateral orifice 1440 is formed is smaller than first radial axis length 14R1. In other words, the lateral orifice 1440 is now nearer or closer to the central point 1420 than the rest of the inner surface 1412 of the sidewall 1430. Drug spray can now exit at an additional distance of D1 from the nozzle to the target region, allowing for a wider coverage of mist around the vomeronasal pit region. In some embodiments, distance D1 can be between ⅓ and 1/12 the length of 14R1, and preferably around 1/6 and 1/10 the length of 14R1.

For clarity, FIG. 14B shows a schematic side-view of the fourth actuator 1400 that better represents the way the sidewall 1430 can include a concave region where the orifice can be formed. The recessed portion 1450 can gradually curve inward in a longitudinal direction from the distal end to the proximal end, so that it has a substantially elongated elliptical shape. This narrowed shape can accommodate, on the opposite side of the sidewall 1430, another recessed portion for the opposite-facing lateral orifice (not shown in FIG. 14B), and the narrowing of the actuator body that leads to distal tip 1452 which includes distal orifice 1416 from which a plume 1454 can be expelled.

From this new position, the lateral orifice 1440 can emit a mist 1460 that is further spaced apart from the target site (e.g., the VNO) than some of the earlier embodiments depicted, allowing for a significantly improved distribution of the drug. Furthermore, because most human nasal passageways include anatomical features that could obstruct or interfere with the operation of the lateral orifice, the recessed portion 1450 can provide clearance between the lateral orifice and any potential anatomical obstructions.

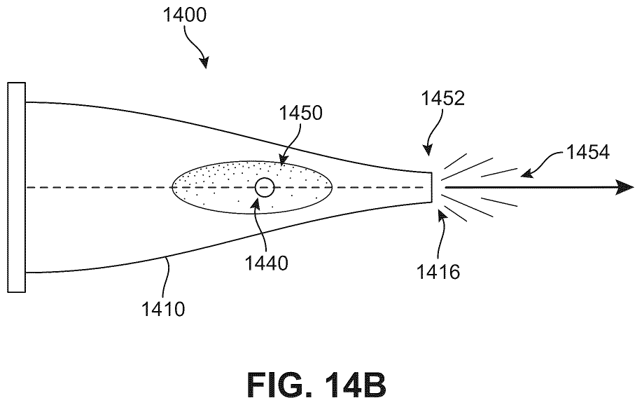

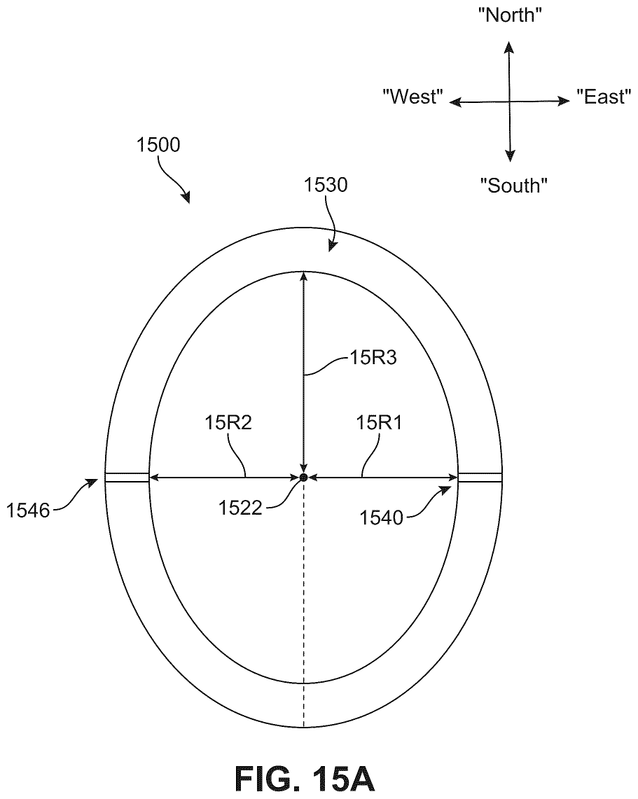

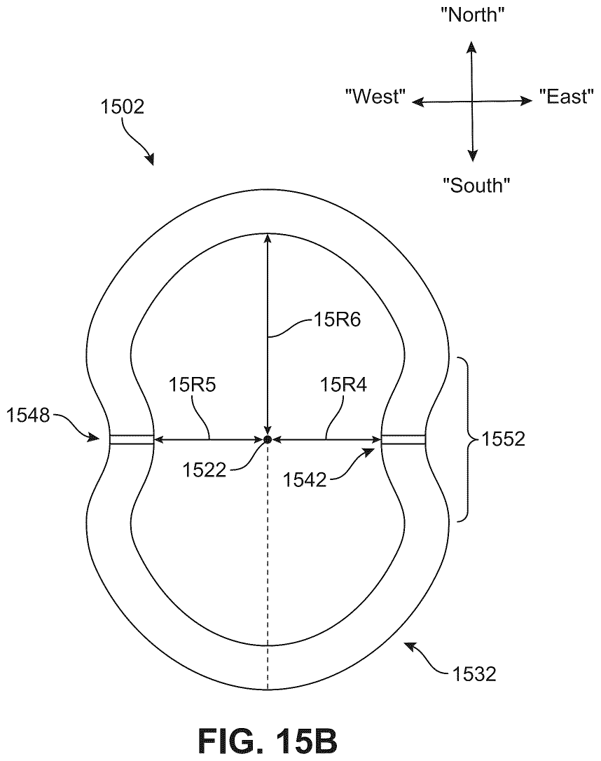

Similarly, in different embodiments, the actuator can include provisions for facilitating comfortable insertion and passage into the human nose. FIGS. 15A and 15B show two examples of actuators configured to complement the structure of a typical human nostril. In FIG. 15B, a schematic cross-sectional view of an embodiment of a fifth actuator 1500 is shown. In this view, the cross- sectional shape as defined by a sidewall 1530 of the actuator can be observed to be substantially oval, oblong, or elliptical. This again brings a first lateral orifice 1540 located at the “Easternmost” position closer to a central point 1520 of the interior chamber (relative to earlier embodiments), at a first distance 15D1. For clarity, a second lateral orifice 1546 is also depicted at the “Westernmost” position, across from the first lateral orifice 1540, at a second distance 15D2 from the central point 1520, where first distance 15D1 and second distance 15D2 are substantially similar or equal, such that the overall cross-sectional shape is also symmetrical relative to a midline extending North-South (i.e., an ellipse's major axis). As a result of this elongated or compressed oval shape, a third distance 15D3 between the central point 1520 and the northernmost position is greater than either of the first distance 15D1 and second distance 15D2.

It can be appreciated that, from these new positions, the two lateral orifices can emit a mist that is further spaced apart from their target site (e.g., the VNO) in each nostril, allowing for a significantly improved distribution of the drug. Furthermore, because most human nasal passageways are more elongated than circular, the proposed shape would be better accommodated in a nasal cavity.

FIG. 15B presents a schematic cross-sectional view of an embodiment of a sixth actuator 1502. In this view, the cross-sectional shape as defined by a sidewall 1532 of the actuator is also narrowed with a generally oval, oblong, or elliptical outline. However, in this case, the sixth actuator 1502 further includes a first recessed portion 1552 and a second recessed portion 1554 (each similar to the recessed portion 1450 described with reference to FIGS. 14A and 14B). This again brings a first lateral orifice 1542 located at the “Easternmost” position closer to a central point 1522 of the interior chamber (relative to earlier embodiments), at a fourth distance 15D4. For clarity, a second lateral orifice 1548 is also depicted at the “Westernmost” position, across from the first lateral orifice 1542, at a fifth distance 15D5 from the central point 1520, where fourth distance 15D4 and fifth distance 15D5 are substantially similar or equal, such that the overall cross-sectional shape is also symmetrical relative to a midline extending North-South (i.e., an ellipse's major axis). As a result of this elongated or compressed oval shape, a sixth distance 15D6 between the central point 1520 and the northernmost position is greater than either of the fourth distance 15D4 and fifth distance 15D5. In addition, it can be appreciated that the fourth distance 15D4 and fifth distance 15D5 of sixth actuator 1502 are both each smaller than the first distance 15D1 and second distance 15D2 of fifth actuator 1500 in FIG. 15A.

It can be appreciated that, from these new positions, the two lateral orifices can emit a mist that is even further spaced apart from their target site (for example, the VNO) in each nostril than some of the earlier depicted examples, allowing for a significantly improved distribution of the drug. Furthermore, because most human nasal passageways are more elongated than circular, the proposed shape would be better accommodated in a nasal cavity.

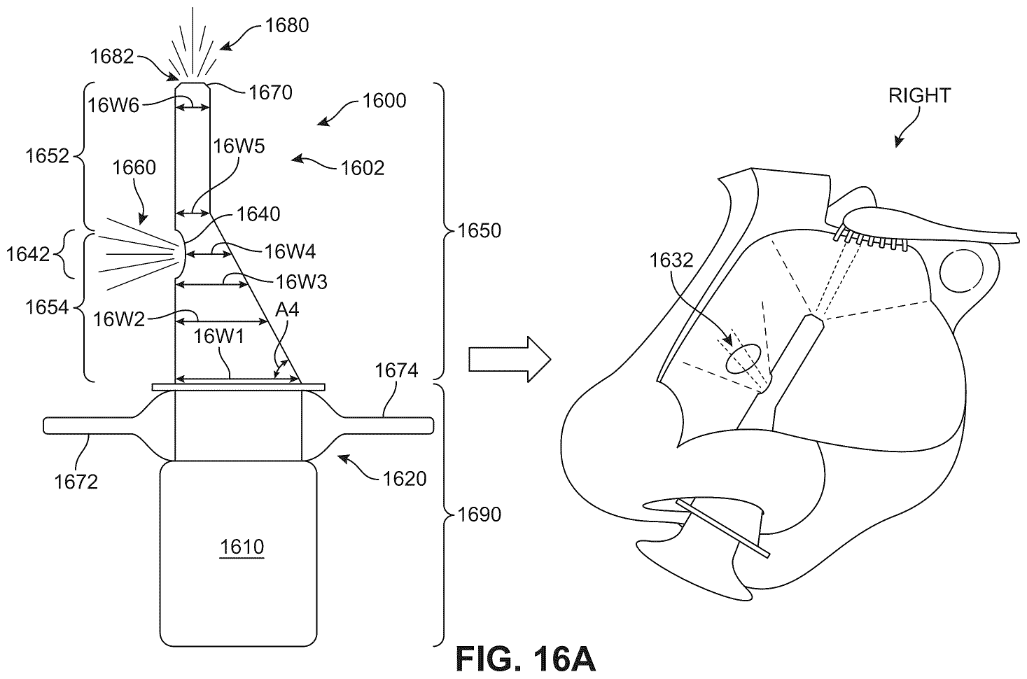

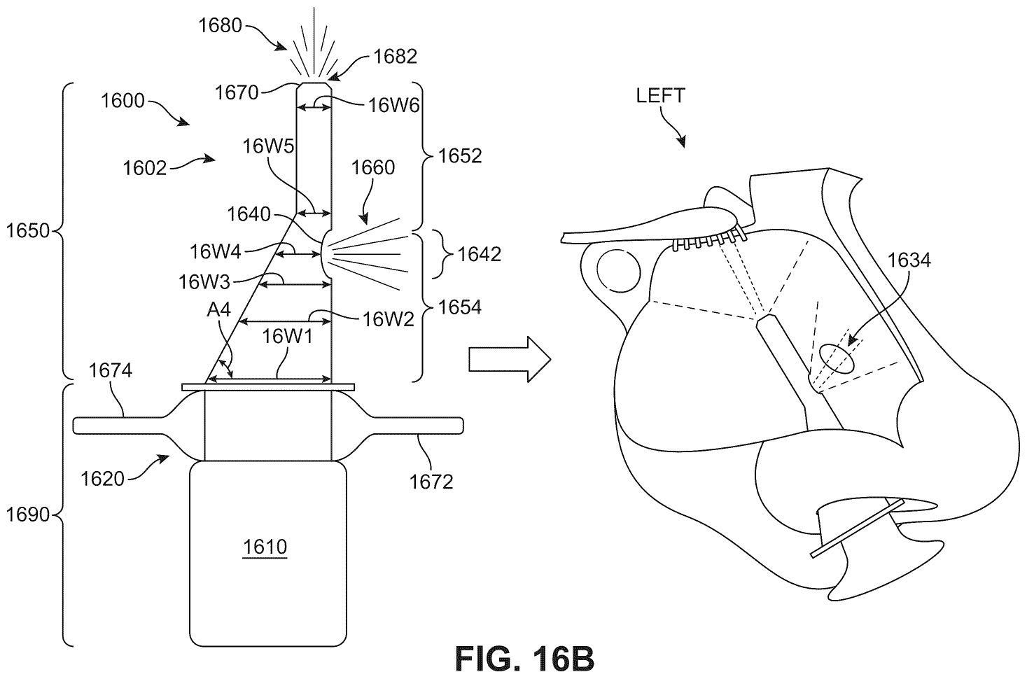

In different embodiments, the proposed device can be configured with a single lateral discharge site that can be used to deliver drugs to the VNO in either nostril, rather than two separate lateral orifices on opposite sides. An example of such an apparatus is presented in FIGS. 16A and 16B. In FIG. 16A, an intranasal device 1600 in a first orientation 1602 is depicted, and in FIG. 16B the same intranasal device 1600 is in a second orientation 1604 that is rotated or flipped 180 degrees relative to a vertical axis (e.g., mirror image). The intranasal device 1600 includes components and features described herein, such as an actuator 1650 (with one lateral orifice 1640 formed in a recessed portion 1642, and one distal orifice 1682 formed at the uppermost end of tip portion 1670) connected to a device assembly 1690 (e.g., comprising pump and vial 1610, a finger-grip push handle 1620, and an optional flange 1630). In this case, the flange 1630 is configured to press or rest against or otherwise come into contact with the nostril once the actuator 1640 is inserted into the nasal cavity, and does in itself not effect or restrict the position in which the device is oriented, in contrast to the embodiments discussed in FIGS. 2-5 above. In FIGS. 16A and 16B, the flange 1630 has a substantially round or oval shape that surrounds a segment of the bottommost portion of actuator 1650, with a wider diameter that prevents a user from pushing the actuator further into the nose than is intended (i.e., the flange is wide enough to bar its entry into the nasal cavity). Furthermore, in different embodiments, the flange 1630 provides a cushioned surface that can abut against the nostril opening to increase comfort for the user.

The intranasal device 1600 also includes a separate component for engaging the drug delivery operations (finger-grip push handlebar 1620). In FIG. 16A, the handlebar 1620 includes two separate prongs, each extending outward on opposite sides of the device, that are sized and dimensioned to receive human fingers. A first prong 1672 protrudes outward from the center of the device assembly 1690 along the same direction as the site of the lateral orifice 1640 (the two are aligned), while a second prong 1674 extends outward in the opposite direction by 180 degrees, thereby restricting placement of the intranasal device 1600 to the only either of the two orientations shown.

In some embodiments, the overall three-dimensional shape of the actuator can be sized and dimensioned to promote its smooth and comfortable to the target depth in each nasal cavity while continuing to target drug delivery to the VNO and olfactory cleft. Rather than a substantially continuous and narrowing tube, the actuator 1650 includes two distinctly shaped sections: a superior portion 1652 and an inferior portion 1654, where the demarcation between the two zones occurs at the upper end of the recessed portion 1642. In different embodiments, the superior portion 1652 is substantially cylindrical in shape, while the inferior portion 1654 is substantially asymmetrical conical or asymmetrical funnel-like shape. One side of the actuator 1650 is substantially linear, while the opposite side is partly linear and partly sloped. This is because, in one embodiment, the inferior portion 1654 can have a substantially right-triangular or sharp letter “D” cross-sectional shape (similar to a shark fin) rising straight up along one side at approximately 90 degrees relative to the horizontal axis, and diagonally upward at an acute angle A1 on its other side, with the actuator base, aligned with the horizontal axis, corresponding to the bottom (third side) of the triangle. In some embodiments, the angle A4 can range between 15 and 40 degrees. In other embodiments, the angle A4 can range between 20 and 30 degrees. In a preferred embodiment, the angle A4 is approximately 25 degrees. This angle has been shown to provide the user with an optimal reference position when orienting the device in the nose. The inferior portion 1654 then merges with the superior portion 1652 at its uppermost end which is also its narrowest region.

As shown in FIG. 16A, the widest part of the funnel is closest to the base of the actuator (e.g., next to flange 1630), with a first width 16W1. Moving upward, the inferior portion 1654 continues to narrow to a smaller second width 16W2. This narrowing continues to a third width 16W3. A fourth width 16W4 is the smallest distance across for the inferior portion 1654, corresponding to the deepest point in the concavity of recessed portion 1642. Upon passing the recessed portion 1642, the inferior portion 1654 ends and the superior portion 1652 continues upward with a fifth width 16W5 until terminating in distal tip 1670. Thus, in one embodiment, fifth width 16W5 and sixth width 16W6 are substantially equal.

It can be appreciated that the first orientation 1602 shown in FIG. 16A is configured for insertion of the actuator into a right-side nostril (from the point of view of the reader), as the “shark fin” or offset funnel shape is unsuitable for insertion into the opposing nostril and can only be accommodated by the geometry of the right-side nostril. This ensures that the lateral orifice 1640 faces toward the septum and mist is correctly directed toward the target region (e.g., a first vomeronasal pit 1632 for the first (right-side) nasal cavity). In FIG. 16B, the intranasal device 1600 has been flipped with respect to a vertical axis. In this second orientation 1604, the actuator 1650 can no longer be inserted into the right-side nostril. Instead, the actuator 1650 can only be inserted into the left-side nostril. Thus, in this orientation, the linear side of the actuator 1650 is again medial with respect to the nasal cavity, and the lateral orifice 1640 faces the toward the septum and mist is correctly directed toward the target region (e.g., a second vomeronasal pit 1634 for the second (left-side) nasal cavity). By this process, the same lateral orifice can be used to deliver the drug to the VNO in the right nostril and the VNO in the left nostril, and the amount of drug that would be wasted is minimized.

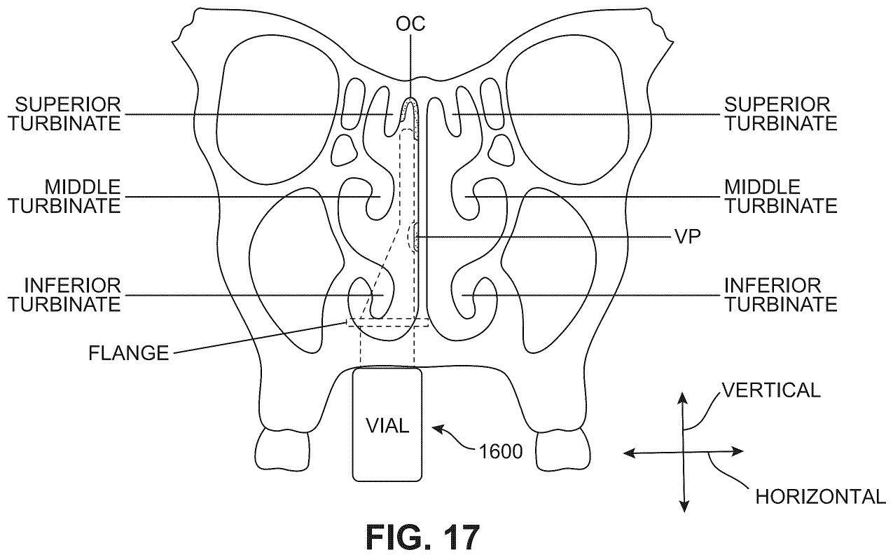

For clarity, FIG. 17 presents a schematic drawing showing an “inserted” coronal view of the intranasal device 1600 into the left-side nostril (S: septum, OC: olfactory cleft, VP: vomeronasal pit within the vomeronasal organ, T: distal tip orifice, and R: lateral orifice formed in the recessed portion of the actuator body). In this example, the lateral orifice (R) is located approximately 20 mm above the actuator's flange, which rests against the opening of the nostril. In this position, drug that emerges from the lateral orifice (e.g., as mist) would cover a 10 mm diameter circle, which corresponds to a known mean size of the vomeronasal pit (VP). Thus, in different embodiments, insertion of the actuator in the nasal passage will position the lateral orifice (R) about the level of the nasal valve (which also represents the lower border of the vomer bone), where the vomeronasal pit (VP) and vomeronasal organ are located. For reference, the nasal septum has a bone upper part (vomer bone) and a cartilage lower part (nasal septal cartilage). The VNO and VNO pit are positioned at the level of the base of the vomer bone (or upper border of the nasal septal cartilage bone). In one example, the mist is outputted with a pressure value of about or no more than 0.7-0.9 Pa.

Moving further upward, FIG. 17 shows the actuator's distal tip orifice (T) being located approximately 20 mm above the lateral orifice (R), thereby positioning the distal tip orifice (T) about 10 mm below the olfactory epithelial lining. This targeted positioning will maximize spraying of the entire olfactory epithelium (both laterally and medially). In different embodiments, the distal tip orifice (T) can emit a spray plume of the selected drug that will have a shape of an inverted short and wide cone. The cone can have a base diameter of around 10 mm, and a height of around 10 mm, thereby covering the medial (septal) region, the top surface, and the lateral surface of the olfactory mucosa. In one example, an impact pressure of about 0.7 Pa is preferable to prevent stimulation of high threshold trigeminal mechanoreceptors, as noted above.

As noted earlier, in different embodiments, the actuator can include materials that facilitate drug delivery and user comfort. In one example, the actuator can comprise of a soft, deformable, and/or flexible material. In some embodiments, the actuator comprises a polymer. In some embodiments, the actuator comprises thermoplastic polyurethane (TPU). In some embodiments, the actuator comprises TPU at grade 65D, 57D, 95A, 90A, 80A, or any combination thereof. In other embodiments, the actuator comprises high-density polyethylene (HDPE). In some embodiments, the actuator includes polyvinyl chloride (PVC). In some embodiments, the actuator comprises a thermoplastic elastomer (TPE). In another embodiment, the actuator comprises styrene-ethylene-butylene-styrene (SEBS). In some embodiments, the actuator comprises low density polyethylene (LDPE). In some embodiments, the actuator comprises silicone (e.g., liquid silicone rubber (LSR)). In some embodiments, the actuator comprises polypropylene. In some embodiments, the actuator includes polytetrafluoroethylene (PTFE), such as for example, Teflon®. In some embodiments, the actuator includes one or more of thermoplastic polyurethane (TPU), high-density polyethylene (HDPE), polyvinyl chloride (PVC), a thermoplastic elastomer (TPE), styrene-ethylene-butylene-styrene (SEBS), low density polyethylene (LDPE), silicone polypropylene, and polytetrafluoroethylene (PTFE). In different embodiments, the actuator can comprise of any combinations of materials thereof, as well as other biocompatible plastics and rubbers.

Furthermore, in different embodiments, the actuator can include provisions for maintaining structural support while accommodating the geometry of the upper nasal passageways. For example, in FIG. 18, an actuator 1820 for an intranasal device 1800 includes a superior portion 1830 and an inferior portion 1840. The actuator 1820 is connected to a device assembly 1890. In this case, the superior portion 1830 can be understood to begin at a site directly above a lateral orifice 1820. At this first time T1, before insertion, the actuator 1810 is in a first configuration 1858, such that the superior portion 1830 continues upward in a generally linear path from the inferior portion 1840, with an angle A5 between a sidewall 1850 and a horizontal axis. In some embodiments, the angle A5 is approximately 90 degrees. In other embodiments, the angle A1 can be between 75 and 90 degrees.

At a second time T2, once a user 1818 inserts the actuator 1810 into their nose 1812, the actuator 1810 can travel through a passageway that is non-linear and, in some cases, rather serpentine. In order to better accommodate its positioning, the actuator 1810 can include different zones of flexibility. This can allow the actuator to deform and flex in order to arrive at the desired insertion distance in a second configuration 1860. For example, in FIG. 18, the superior portion is more curved (at an angle A2) than it was earlier, such that angle A6 is smaller or narrower than angle A5.

More specifically, in some embodiments, the superior portion 1830 includes materials that are more flexible or elastic than the materials of the inferior portion 1840, which are relatively more rigid or hard. In other words, the superior portion 1830 can have a first rigidity that is less than a second rigidity of the inferior portion. Furthermore, in some embodiments, the materials used can be distributed to form a rigidity gradient that is greatest/most rigid toward the base of the actuator and least rigid/softest at the distal tip 1816. This is represented in the drawing by a density of stippling that is heaviest near the distal tip 1816, reflecting a more flexible material, and gradually becomes less dense moving downward toward the base of the actuator 1810 until there is no stippling, representing the greatest region of rigidity in the actuator. Thus, the material around the distal tip 1816 can include a third rigidity, and a part of the superior portion 1830 below the distal tip 1816 can have a fourth rigidity that is greater than the third rigidity. Similarly, the lateral orifice can be formed in a part of the inferior portion 1840 with a fifth rigidity, and a part of the inferior portion 1840 further below can have a sixth rigidity that is greater than the fifth rigidity. This approach allows adaptation of the actuator to the curvature of the nasal septum. In addition, the relatively “softer” or more flexible material in the superior portion can minimize the likelihood that the sidewall itself might put pressure on the nasal tissues and stimulate trigeminal pain receptors that might trigger an undesirable defensive reflex reaction.

### XI. Actuator Discharge Orifices

The following section offers additional details directed to embodiments in which the discharge orifices of the actuator can be modulated to influence drug delivery performance. Turning to FIG. 19, a seventh actuator 1900 is depicted including a lateral orifice 1916 provided in a sidewall 1932 and a distal orifice 1930 provided at an opening of the distal tip (not to scale). In different embodiments, each orifice can be associated with different structural characteristics that impact its output of drug. In this example, the distal orifice 1930 includes a nozzle swirl chamber 1922 which is configured to emit a spray plume 1922. In some embodiments, for a dose of 100 microliter per actuation, the spray of the swirl chamber 1922 can have an initial velocity of about 13 m/s, spraying a total volume of 100 microliters in approximately 100 ms.

In contrast, the lateral orifice 1916 includes a soft mist nozzle 1910 (e.g., Medspray® soft mist nozzle) that comprises a plurality of micro-pores, also referred to herein as a “micro-grid” (e.g., see FIGS. 28-32 below). In some embodiments, where there are 48 pores that are each 4 micrometers in diameter, for a dose of 45 microliters per actuation, the spray from the soft mist nozzle 1910 is produced at a much lower initial velocity of about 0.1, 0.2, 0.3, 0.4, 0.5, 0.6, and 0.7 m/s, (for example with sprays of 45 microliters in 500 ms). In some embodiments, the initial velocity can be lower than 0.4 m/s to further reduce the likelihood of activating trigeminal nerve nociceptors, which as noted herein is undesirable. Furthermore, the soft mist nozzle 1910 produces much smaller droplets than the standard swirl nozzle. In addition, while the standard swirl nozzle produces a short burst of droplets, all traveling upwards in straight trajectories, the soft mist nozzle produces droplets in a turbulent droplet cloud. In some embodiments, the emission of the drug as a soft mist may also extend the duration of action due to the delivery of the drug at lower velocities.

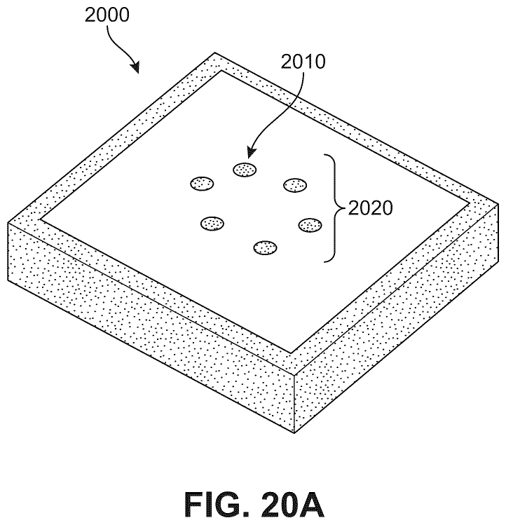

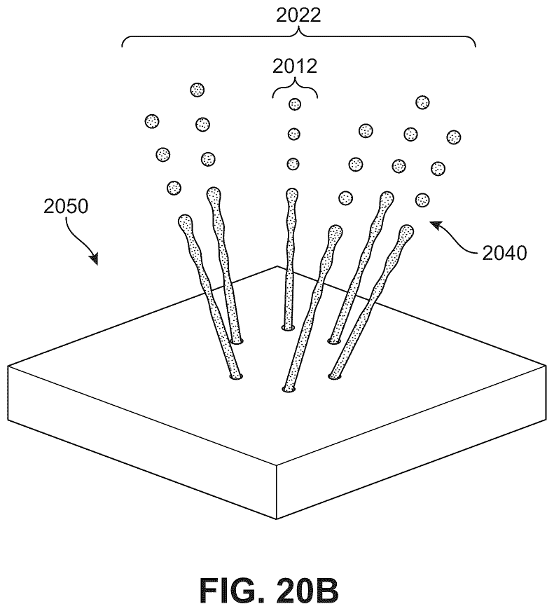

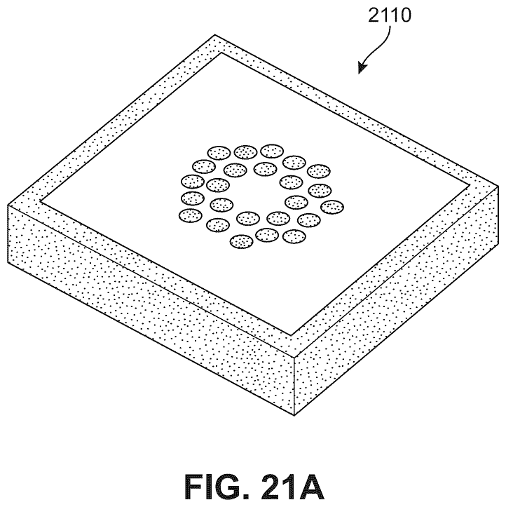

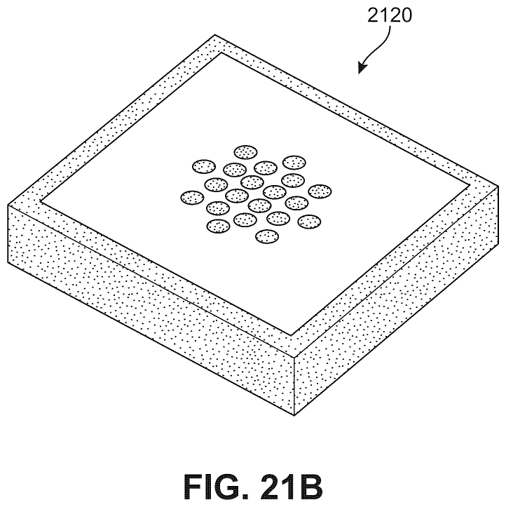

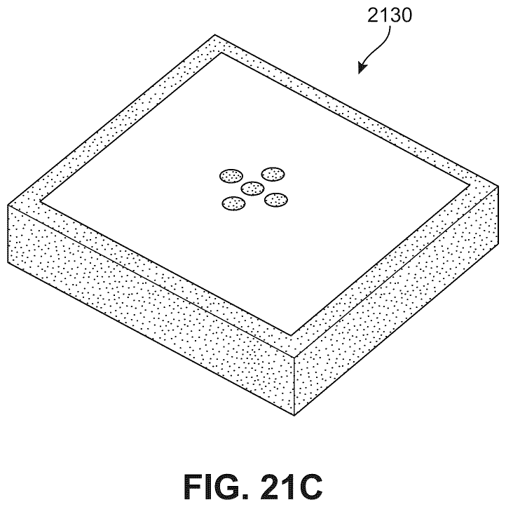

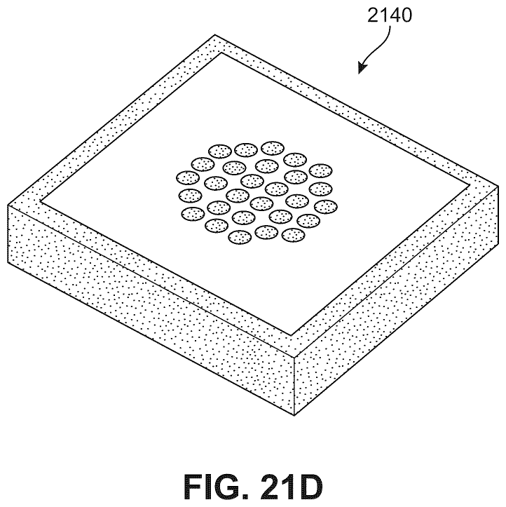

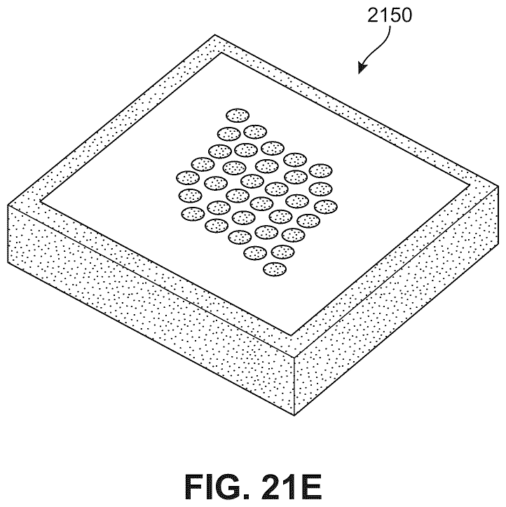

In different embodiments, the proposed intranasal devices can incorporate lateral orifices with varying properties to modulate the output of the soft mist, the coverage of the spray, and its range. Referring to FIGS. 20A-21E, for purposes of illustration, several examples of lateral orifice soft mist nozzle micropore configurations that can be used in the actuator are shown. In FIG. 20A, a first micropore configuration 2000 is depicted, including a first set of micropores 2010 (six micropores) in a first arrangement 2020 (circular). FIG. 20B is a schematic diagram showing how fluid traveling through the micropores can be transformed into mist particles 2040. Each pore generates an individual exiting fluid stream 2012, which collectively form a mist spray 2022. Other non-limiting variations of the micro-pore arrangements that can be used are presented in FIGS. 21A-21E, including a second configuration 2110, a third configuration 2120, a fourth configuration 2130, a fifth configuration 2140, and a sixth configuration 2150. As illustrated, the number of micro-pores can be modulated to adjust the output coverage, volume, pressure, and volume of the drug.

In different embodiments, the proposed devices can also include provisions for fine-tuning the alignment of the drug delivery plume route relative to the actuator's sidewall. Some examples of this approach are presented in FIGS. 22A-22C. In previous examples, the distal boundary and the proximal boundary of each channel were parallel to one another. In other words, the opening along the sidewall could be cut to route the plume in a generally transverse “cut” relative to the sidewall's orientation. However, in some other cases, it may be desirable to “funnel” the drug through the thickness of the sidewall using other orientations. For example, in FIG. 22A, a top-down cross-sectional view of a portion of an eighth actuator 2210 is depicted. In this cross-section, an interior chamber 2246 of the eighth actuator 2210 is shown, bounded by a continuous sidewall 2240 that serves as the actuator's laterally oriented exterior boundary and substantially encloses the chamber 2246. The actuator body sidewall 2240 includes an exterior surface 2242 and an opposite-facing interior surface 2244, separated by a substantially uniform thickness.

As shown in FIG. 22A, upon actuation and the release of drug into the chamber 2246, the drug can travel in a distal direction. Fluid traveling along the length of the first actuator 2210 arrives near to interior surface 2244 of the sidewall 2240. During this process, some fluid can enter a first end 2216 of a first outlet 2212 formed in the interior surface 2244 and travel outward until exiting a second end 2214 of the first outlet 2212 provided in the exterior surface 2242. This process also occurs throughout several of the embodiments that have been earlier described. However, in FIG. 22A, the diameter or size of the first end 2216 is smaller than the diameter or size of the second end 2214, producing an inverse funnel effect whereby the drug is first concentrated and then distributed across a wider array (per a relatively large spray plume geometry angle A12). Furthermore, beyond this variation in size, the orientation of the “cuts” for the ends can differ. For example, in this case, a proximal cut of the first outlet 2212 is oriented “backward” or toward a proximal direction as it moves from the interior chamber 2246 to the external environment, while a distal cut is oriented in an opposing direction, such that both sides of the channel are generally symmetrical relative to a central lateral axis (shown by a dotted line in FIG. 22A) even though the two edges extend in different directions.

As a second example, FIG. 22B depicts a top-down cross-sectional view of a portion of a ninth actuator 2220. In this cross-section, an interior chamber 2256 of the ninth actuator 2220 is shown, bounded by an actuator body sidewall 2250 that serves as the actuator's exterior boundary and substantially encloses the chamber 2256. The actuator body sidewall 2250 includes an exterior surface 2252 and an opposite-facing interior surface 2254, separated by a substantially uniform thickness.

As shown in FIG. 22B, upon actuation and the release of drug into the chamber 2256, the drug can travel in a substantially distal direction. Fluid traveling along the length of the ninth actuator 2220 arrives near to interior surface 2254 of the sidewall 2250. During this process, some fluid can enter a first end 2226 of a second outlet 2222 formed in the interior surface 2254 and travel outward until exiting from a second end 2224 of the second outlet 2222 provided in the exterior surface 2252. Similar to FIG. 22A, the diameter or size of the first end 2226 is smaller than the diameter or size of the second end 2224 in FIG. 22B. However, the orientation of the sides of the channel have been modified to allow for a narrower spray plume geometry angle of A7 that is pushed forward, or in a distal direction. For example, in this case, a proximal cut of the second outlet 2222 is oriented or tilted forward, or toward a distal direction as it moves from the interior chamber 2256 to the external environment. In addition, similar to FIG. 22A, the distal cut is oriented in a direction that is also angled forward. This leads to a diagonal tunnel where the two edges extend outward and up, causing the drug to be routed along a more anterior and superior path than the example that was shown in FIG. 22A. In different embodiments, this structural arrangement for the lateral orifice of the actuator will deliver a mist to the vomeronasal organ (VNO) that is diagonal instead of normal to the septum wall. This will also necessarily change the shape of the VNO sprayed area from a circle to an ellipsoid, which will improve the likelihood of spraying the entire vomeronasal pit in a larger range of human noses that can differ according to sex and ethnicity. In such embodiments, the lateral orifice can then be situated slightly lower along the actuator length (that is, further spaced apart from the distal tip), so that its target (diagonally upward) remains the VP. In some embodiments, where the lateral orifice is directed upward around 10-20 degrees, the lateral orifice(s) can be re-located to between approximately 15-19 mm from the nostril and/or flange, and 21-25 mm from the distal tip.

An additional example is presented in FIG. 22C by an illustration of a top-down cross-sectional view of a portion of a tenth actuator 2230. In this cross-section, an interior chamber 2266 of the tenth actuator 2230 is revealed, as bounded by a continuous actuator body sidewall 2260 that serves as its exterior boundary and substantially encloses the chamber 2266. The actuator body sidewall 2260 includes an exterior surface 2262 and an opposite-facing interior surface 2264, separated by a thickness that in this case can vary throughout different regions of the actuator body.

As shown in FIG. 22C, upon actuation and the release of drug into the chamber 2266, the drug can flow in a substantially distal direction. Fluid traveling along the length of the tenth actuator 2230 arrives near to interior surface 2264 of the sidewall 2260. During this process, some fluid can enter a first end 2236 of a third outlet 2232 formed in the interior surface 2264 and travel outward until exiting from a second end 2234 of the third outlet 2232 formed in the exterior surface 2262.

As noted with respect to FIG. 22A, the diameter or size of the first end 2236 is smaller than the diameter or size of the second end 2234 in FIG. 22C, producing a funnel effect. In part, this funnel effect is created by the relative orientation of the sides of the channel that dip toward one another (e.g., forming a volcano-like geometry), and resulting in a wider spray plume geometry angle. For example, in this case, a distal cut of the third outlet 2232 is oriented or tipped “forward” or toward a distal direction as it moves from the interior chamber 2266 to the external environment. In addition, the proximal cut is oriented in “backward” or toward a proximal direction as it moves from the interior chamber 2266 to the external environment.

Thus, it can be appreciated that the actuators described herein can offer custom spray configurations that can target specific regions and drug dispersal patterns in the nasal cavity. This customization can be realized in part by variations in the angle and orientation of each channel. Furthermore, in some embodiments, there may be a need to extend or magnify the effects of the channel's structural characteristics on the emerging fluid. In such cases, a length of the “tunnel” provided by one or more of the apertures can be extended to allow the orientation and/or shape of the aperture to more strongly influence the fluid dynamics of the drug that passes through the outlet. As one non-limiting example, in FIG. 22C, a different, second portion of the sidewall 2260, in this case across from the portion through which the third outlet 2232 was formed, can be seen to also include a channel, referenced here as fourth outlet 2238. However, in contrast to the third outlet 2234 which was cut through a sidewall portion with a first thickness T1, the fourth outlet 2238 has been cut through a sidewall portion with a second thickness T2 that is larger than first thickness T1. In some embodiments, T2 can be 2-3 times as thick as T1. As a result, the channel that extends through the sidewall here has a significantly greater depth, and so can influence the fluid dynamics of the drug passing through to a greater extent than the channel with the shallower depth. In some embodiments, the thickness can be varied in a more nuanced fashion, such that the actuator includes one or more “bumps” where the thickness is increased in only those localized sidewall regions that surround one of the outlets, and/or regions where the material is thinner so that the channels are shorter, thereby reducing the amount of material and weight required by the actuator while modulating the length of the channel.

For clarity, a magnified view is included depicting the fourth outlet 2238 in greater detail. The funnel shape is more apparent, whereby a first length L1 on the interior side is less than a second length L2 of the exterior side. In some embodiments, first length L1 can be between ⅙ and ⅚ the size of second length L2, and preferably around ⅓. In addition, it can be appreciated that the angle at which a channel is cut can be varied to modulate the direction of the outputted spray. In this example, the proximal cut extends from the interior surface 2264 at an acute angle A8, and the distal cut extends from the interior surface at an acute angle A9. In some embodiments, the angles A8 and A9 can be equal, so that the channel width is consistent across its length. In different embodiments, the angles A8 and/or A9 can be approximately 10-50 degrees. In other embodiments, as shown in FIG. 22C, the angles A8 and A9 can differ. For example, in FIG. 22C, angle A8 is larger than angle A9. As a result, the length of the proximal cut is also shorter than the length of the distal cut.

In different embodiments, depending in part on the dispenser mechanism selected, there may be variation in the parameters of the pharmaceutical expelled through each of the outlets, such as the speed of the particles and their density. These parameters can be adapted to accommodate the condition being treated. It should be appreciated in different embodiments, that both the primary outlets (for example, the distal outlet) and the secondary outlets (for example, the lateral openings formed in the thickness of the sidewall) can be configured passive outlets configured to allow for consistency in the pressure that is experienced by the user during actuation. In other words, the speed of secondary flows of fluid exiting the lateral openings can generally match the speed of the primary flow of fluid that exits the distal opening, if both provide passive flow paths. However, it can be understood that with the variations in the structural characteristics of each opening as described herein, the resultant speed of the flow can be affected.

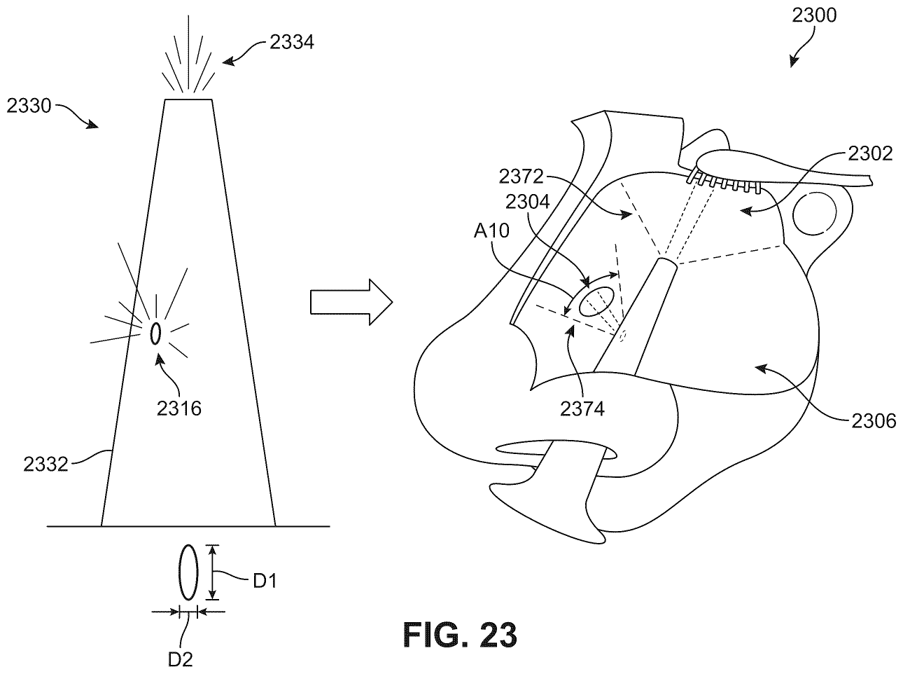

In some embodiments, the shape of the lateral orifice can also be modified to optimize or refine delivery to the VNO or other target area. For example, in FIG. 23, an eleventh actuator 2310 is presented in an isolated side view. The eleventh actuator 2310 includes both a first outlet 2314 and a second outlet 2316. The second outlet 2316 includes a through-hole aperture formed in a sidewall 2312 of the actuator body. In this example, the aperture comprising the second outlet 2316 has a substantially elliptical or oval shape, with a major axis-diameter (D1) that is greater than a minor axis-diameter (D2). When the eleventh actuator 2310 (as part of an intranasal device, not shown here) is inserted by a user 2300 into a nasal cavity 2306 and actuated, a first plume 2372 is ejected axially from the opening at the distal tip, targeting a first region 2302 (in this case the olfactory cleft). In addition, a second plume 2374 is ejected laterally from the oval aperture on the sidewall corresponding to the second outlet 2316, targeting a second region 2304 (in this case, the vomeronasal pit). The position of the second outlet 2316 and its elongated shape allows the second plume 2374 to deliver a dose of the drug to the VNO with rapid overall clearance while a spray plume geometry angle of A10 permits the spraying of a broader area.

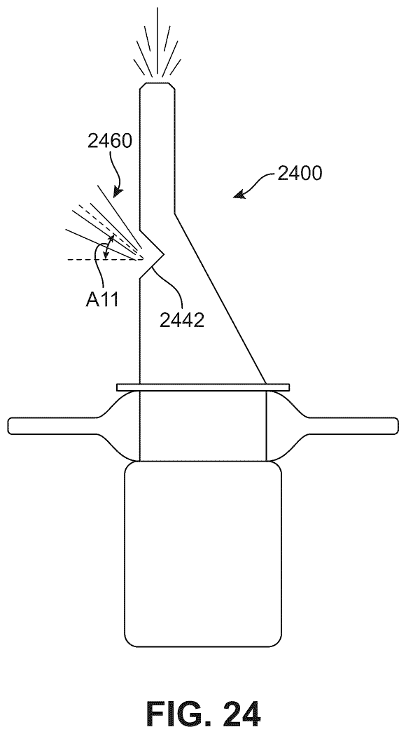

In different embodiments, the oval/elliptical shape can be manifested not only as a single opening, but by the spatial arrangement of micro-pores in the cases where a soft mist nozzle is employed (e.g., see FIG. 21E). Furthermore, as noted earlier with respect to FIGS. 22A-22C, the orientation of the passage can be changed, and in the case where the lateral orifice is comprised of micro-pores, this can be provided by each pore having diagonally oriented cuts and/or the pores being incorporated at different parts of the recessed portion. One example of this is shown in FIG. 24, where lateral orifice 2440, made up of a plurality of micro-pores, is tilted in an upward direction. Thus, when the soft mist spray is generated, the mist will travel out in a diagonally upward direction tilted by an acute angle A11 relative to a lateral axis (axis that would come straight out). In some embodiments, angle A11 is between 5 and 40 degrees, and preferably around 10 degrees. This technique can be used to further refine the aim of the spray to a selected target area.

### XII. Example Methods, Systems, and Devices

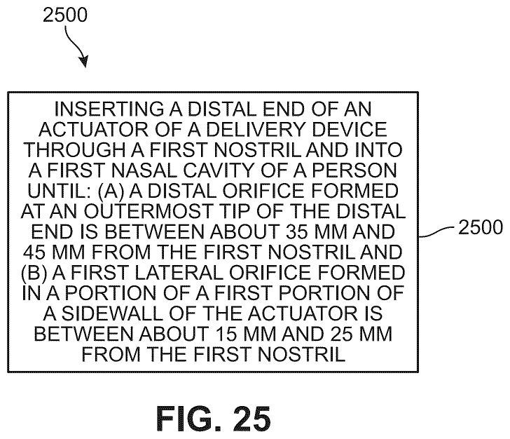

FIG. 25 is a flow chart illustrating an embodiment of a method 2500 of delivering drugs to multiple regions of a subject's nasal cavity including the olfactory cleft and the vomeronasal organ (VNO). The method 2500 includes a step 2510 of inserting a distal end of an actuator of a delivery device through a first nostril and into a first nasal cavity of a person until: (a) a distal orifice formed at an outermost tip of the distal end is between about 35 mm and 45 mm from the first nostril; and (b) a first lateral orifice formed in a portion of a first portion of a sidewall of the actuator is between about 15 mm and 25 mm from the first nostril.

In other embodiments, the method may include additional steps or aspects. In one example, the method 2500 further includes a step of positioning the delivery device so that a flange attached to a lowermost portion of the actuator abuts the first nostril. In some embodiments, the method 2500 can include a step of delivering, to the VNO, a first metered dose of a first pherine compound via the first lateral orifice.

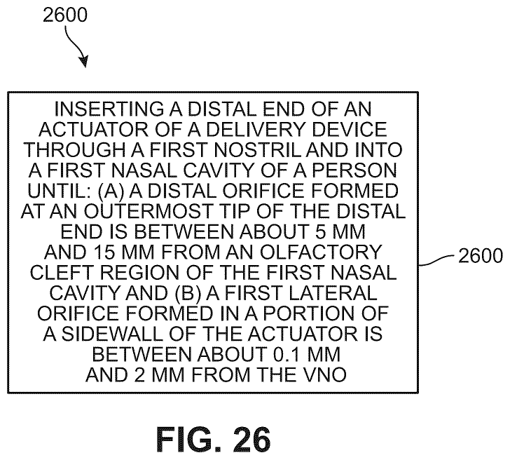

Other methods may be contemplated within the scope of the present disclosure. For example, FIG. 26 is a flow chart illustrating an embodiment of a method 2600 of delivering drugs to multiple regions of a nasal cavity including the olfactory cleft and the vomeronasal pit (VP). The method includes a step 2610 of inserting a distal end of an actuator of a delivery device through a first nostril and into a first nasal cavity of a person until: (a) a distal orifice formed at an outermost tip of the distal end is between about 5 mm and 15 mm from an olfactory cleft region of the first nasal cavity; and (b) a first lateral orifice formed in a portion of a sidewall of the actuator is between about 0.1 mm and 2 mm from the VP.

In other embodiments, the method may include additional steps or aspects. In one example, the method 2600 further includes a step of orienting the actuator so that the first lateral orifice is facing toward a septum of the first nasal cavity. In some embodiments, the method 2600 also includes a step of delivering, to the VP, a first metered dose of a first pherine compound via the first lateral orifice.

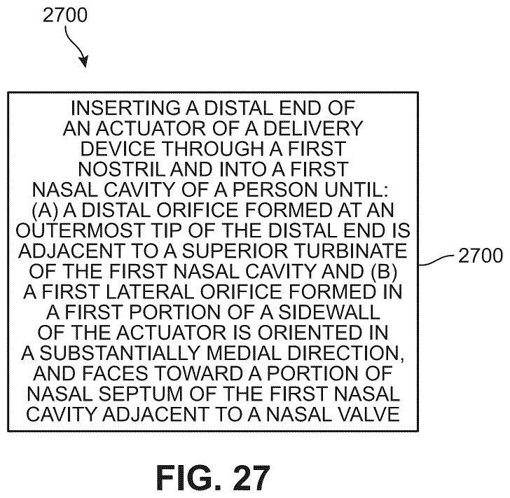

Other methods may be contemplated within the scope of the present disclosure. For example, FIG. 27 is a flow chart illustrating an embodiment of a method 2700 of delivering drugs to multiple regions of a nasal cavity including the olfactory cleft and the vomeronasal organ (VNO). The method 2700 includes a step 2710 of inserting a distal end of an actuator of a delivery device through a first nostril and into a first nasal cavity of a person until: (a) a distal orifice formed at an outermost tip of the distal end is adjacent to a superior turbinate of the first nasal cavity; and (b) a first lateral orifice formed in a first portion of a sidewall of the actuator is oriented in a substantially medial direction, and faces toward a portion of nasal septum of the first nasal cavity adjacent to a nasal valve.

In other embodiments, the method may include additional steps or aspects. In one example, the first portion is recessed relative to second portion of the sidewall surrounding the first portion. In some embodiments, the method 2700 also includes a step of delivering, to the VNO, a first metered dose of a first pherine compound via the first lateral orifice.

Other methods may be contemplated within the scope of the present disclosure. For example, in some embodiments, a method of delivering pherines to multiple regions of a nasal cavity including the olfactory cleft and the vomeronasal organ (VNO) is disclosed. The method includes a step of delivering, to the VNO, a first metered dose of a first pherine compound through a first lateral orifice provided in an actuator body of a delivery device, the first pherine compound exiting the first lateral orifice as a mist. In other embodiments, the method may include additional steps or aspects. In one example, the method can also include a step of delivering, to the distal cleft, a second metered dose of the first pherine compound through a distal orifice formed at a distal tip of an actuator body of a delivery device, the first pherine compound exiting the distal orifice as a plume. In some embodiments, the first lateral orifice comprises a plurality of micro-pores.

Other methods may be contemplated within the scope of the present disclosure. For example, in some embodiments, a method of delivering drugs to multiple regions of a nasal cavity including the vomeronasal organ (VNO) is disclosed. The method includes a step of delivering a first metered dose with an impact pressure of no more than about 0.8 Pascals via a lateral orifice provided in an actuator body of a delivery device to the VNO of a first nasal cavity, thereby covering surfaces of the vomeronasal organ of the first nasal cavity while preventing activation of a majority of high threshold trigeminal mechanoreceptors in the first nasal cavity. In different embodiments, embodiments, the impact pressure is no more than about 0.1, 0.2, 0.3, 0.4, 0.5, 0.6, 0.7, 0.8, 0.9, and 1 Pascal. In other embodiments, the method may include additional steps or aspects. In one example, the method can also include a step of delivering a second metered dose with an impact pressure of no more than 0.8 Pascals via a distal orifice formed at an outermost tip of the distal end to an olfactory cleft region of a first nasal cavity, thereby covering surfaces of an olfactory mucosa region of the first nasal cavity while preventing activation of a majority of high threshold trigeminal mechanoreceptors in the first nasal cavity. In some embodiments, the first metered dose is emitted as a mist, and the second metered dose is emitted as a plume.

As described herein, an actuator for an intranasal spray device can include a plurality of orifices, where the relative position of each orifice along the body of the actuator is carefully selected to align with a target region in the nasal cavity. For example, the distal orifice can be used to deliver drugs in a plume to the olfactory cleft, while the lateral orifice can direct drugs via mist to the vomeronasal organ (VNO). In some embodiments, the lateral orifice can be located on the actuator so that when the actuator is inserted correctly into a human nostril, the lateral orifice is directly aligned with and facing the VNO area of the septum, and any drug emitted from the lateral orifice is laterally oriented to cover that site. In some embodiments, the lateral orifice alternatively can be located on the actuator so that when the actuator is inserted correctly into a human nostril, the lateral orifice is a few millimeters lower than the VNO area (for example, 0-5 mm, preferably 2-4 mm) and includes structural features that orient the outlet diagonally relative to the septum, so that the output is pushed upward and medially toward the VNO area for coverage at the VNO. In some embodiments, insertion of the device actuator is facilitated by a flange grip that only permits insertion of the actuator into the first nasal cavity while the actuator is in the first orientation. In another embodiment, the actuator includes a substantially “D” cross-sectional shape so that only insertion of the actuator into the first nasal cavity can only occur while the actuator is in the first orientation. In some embodiments, the actuator is provided with an outermost/exterior sidewall that substantially encloses an interior chamber. An upper portion of the sidewall includes a first material, and a lower portion of the sidewall includes a second material, and the first material is more elastic than the second material. In some embodiments, this difference in material elasticity can occur gradually down the length of the actuator so that there is a gradient of rigidity in the material. In different embodiments, the lateral orifice is situated above a majority of the inferior turbinate when properly/correctly positioned within a nasal cavity. In some embodiments, the lateral orifice is situated below the middle turbinate when properly/correctly positioned within a nasal cavity. In different embodiments, the lateral orifice is situated near and/or across from the nasal valve when properly inserted into a nasal cavity. In some embodiments, the lateral orifice is oriented with a spray angle between 5 and 25 degrees relative to the distal orifice. In some embodiments, the lateral orifice is formed in a section and/or portion of the actuator that is dipped, shallow, concave, recessed, and/or otherwise curves inward relative to the majority of the sidewall surface that surrounds the recessed section. In some embodiments, the actuator includes two lateral orifices, and one lateral orifice faces medially toward the septum and the other lateral orifice faces laterally toward the lateral wall when the actuator is properly/correctly positioned within a nasal cavity. In some embodiments, the actuator includes a round cross-sectional shape in a horizontal plane, and the two lateral orifices are arranged at or about 180 degrees apart from one another. In some embodiments, the lateral orifice is made up of a plurality of micro-pores (soft mist nozzle). In some embodiments, the micro-pores can include 2 or more openings that direct fluid flow toward the VP.

Other methods may be contemplated within the scope of the present disclosure. For example, in some embodiments, a method for treating a mental disorder is disclosed. The method includes a step of intranasally administering to an individual in need thereof an effective dose of a pherine compound, where the pherine compound is delivered to a region of a nasal cavity that includes the vomeronasal organ (VNO). In other embodiments, the method may include additional steps or aspects. In different embodiments, the pherine compound is selected from one of fasedienol, itruvone, PH80, PH15, and PH284. In some embodiments, the mental disorder is one of social anxiety, separation anxiety, generalized anxiety, obsessive-compulsive symptoms, sound phobias, dysmenorrhea, and depression.

In different embodiments, the disclosure also provides for a therapeutic device for treating disorders, comprising an intranasal spray device with an actuator configured with two orifices for directing delivery of a pherine-based formulation to both an olfactory cleft and a vomeronasal organ in a subject's nasal cavity.

In different embodiments, the disclosure also provides for a kit of parts for treatment of one or more disorders, the kit comprising: a vial including a pherine-based formulation; and an actuator in fluid communication with the vial, the actuator including two orifices, where the actuator is configured to deliver a first metered dose of the pherine-based formulation to a vomeronasal organ in a subject's nasal cavity, and a second metered dose of the pherine-based formulation to an olfactory cleft in the subject's nasal cavity.

In different embodiments, the disclosure also provides for a kit that includes at least one pherine in a labeled package, wherein application of the at least one pherine occurs by an intranasal spray device inserted into a nasal cavity, and the label on the package indicates that the at least one pherine can be used in treatment of at least one disorder.

In different embodiments, the disclosure also provides for an actuator for an intranasal drug delivery device. The actuator can include: a base portion that is in fluid communication with a reservoir of the intranasal drug delivery device; an axially-oriented distal orifice provided in a distal tip portion of the actuator that is in fluid communication with an environment external to the interior chamber; and a first lateral orifice formed through a thickness of a first portion of a sidewall of the actuator, where the first lateral orifice is also in fluid communication with the environment external to the interior chamber. In different embodiments, the first portion is recessed relative to a second portion of the sidewall that surrounds the first portion. In some embodiments, the first lateral orifice comprises a plurality of micro-pores.

In different embodiments, an intranasal nasal drug delivery device is disclosed. The intranasal drug delivery device can include: a reservoir that includes a pherine composition selected from the group consisting of one or more of fasedienol, itruvone, PH80, PH15, and PH284; and an actuator in fluid communication with the pherine composition. The actuator includes an actuator body comprising of a tubular sidewall extending from a base portion to a distal tip portion. In addition, the distal tip portion can include a distal orifice configured to direct a portion of the pherine composition onto nasal chemosensory receptors associated with the mucosa in the olfactory cleft, and the tubular sidewall can include a lateral orifice configured to direct a portion of the pherine composition onto nasal chemosensory receptors associated with the vomeronasal organ (VNO). In some embodiments, the actuator body includes an inferior portion and a superior portion, and the inferior portion includes a substantially D-shaped cross-section that constrains insertion of the actuator into a nasal cavity to one of two orientations. In another embodiment, the lateral orifice comprises a plurality of micro-pores configured to generate a mist when emitting the pherine composition.

In some embodiments, the disclosure also provides for an actuator assembly for an intranasal drug delivery device. The actuator assembly can include: a first actuator including a first axially-oriented distal orifice provided in a first distal tip portion of the first actuator, and a first lateral orifice formed through a first thickness of a first sidewall; and a second actuator including a second axially-oriented distal orifice provided in a second distal tip portion of the second actuator, and a second lateral orifice formed through a second thickness of a second sidewall.

In some embodiments, the first actuator and the second actuator are both in fluid communication with a single reservoir. In another embodiment, the first lateral orifice and the second lateral orifice face toward one another.

In one embodiment, the disclosure provides an actuator for a nasal spray device, including an actuator having multiple discharge orifices, that is capable of controlling both the directionality and pressure of a nasal spray in order to optimally deliver a pherine drug to at least the primary and secondary target areas of the olfactory chemosensory epithelium, and preferably, also to the tertiary target areas of the olfactory chemosensory epithelium in the nasal cavity.

In another embodiment, by utilization of the disclosed nasal spray devices, the present disclosure contemplates ready-for-administration pherine drug compositions in the reservoir of the device, these pherines including those mentioned in the patents described in the BACKGROUND, particularly including fasedienol (PH94B), itruvone (PH10), 16α, 17α epoxyestr-4 en-10β ol-3 one (PH80), estra-1,3,5(10), 16-tetraen-3-yl acetate (PH15), and 19-norpregna-1,3,(10)-trien-3-ol (PH284). The disclosure further contemplates a package or kit that combines such a ready-for-administration device together with appropriate packaging and instructions for its use.

In different embodiments, the disclosure provides an intranasal drug delivery device comprising a dispenser assembly including a reservoir; an actuation mechanism including an actuator, the actuator including a base portion, a distal tip portion, and an interior chamber enclosed by a sidewall. In some embodiments, the base portion is attached to the dispenser assembly and is in fluid communication with the reservoir, and the distal tip portion includes a first axial discharge orifice that is in fluid communication with an environment external to the interior chamber. In one embodiment, the sidewall includes a first lateral discharge orifice formed through a thickness of the sidewall that is in fluid communication with the environment external to the interior chamber.

In one embodiment, the disclosure provides an actuator assembly for an intranasal drug delivery device. The actuator can include: an actuator body that extends longitudinally from a base portion to a distal tip portion, the actuator body including a sidewall; a first discharge orifice formed in the distal tip portion; and a second discharge orifice formed in a first laterally-oriented region of the sidewall, each of the first discharge orifice and the second discharge orifice providing fluid communication between an interior of the actuator body and an environment external to the interior.

In another embodiment, the disclosure provides an intranasal nasal drug delivery device that includes: a reservoir that includes a pherine composition selected from the group consisting of one or more of fasedienol, itruvone, PH80, PH15, and PH284; and an actuator including a lateral sidewall and a distal tip. In some embodiments, the distal tip of the actuator includes or more distal orifices configured to direct a portion of a pharmaceutical substance onto nasal chemosensory receptors associated with the mucosa in the dorsal recess nasal cleft, and the lateral sidewall of the actuator includes one or more lateral orifices configured to direct a portion of the pharmaceutical substance onto nasal chemosensory receptors associated with the vomeronasal organ.

In some embodiments, the disclosure provides a method of delivering a prophylactic or therapeutic nasal spray to the chemosensory mucosa, comprising the administration of a spray of pharmaceutical substance with the intranasal drug delivery device of any of the foregoing embodiments and examples.

### XIII. Intranasal Device Pump Assembly

FIG. 28 shows a schematic diagram of an alternative embodiment of an intranasal device 2800. Similar to other embodiments, intranasal device 2800 includes a vial, bottle, or reservoir 2880 that is attached a pump assembly (“pump”) 2882. The inner cavity of reservoir 2882 is in fluid communication with portions of pump assembly 2882. Collectively, for purposes of reference, the pump 2882 and reservoir 2880 can comprise a main body or “intranasal device main body” or simply “main body” 2830.

Pump assembly 2882 can include provisions to dispense precise quantities of drug formulation 2884 contained within reservoir 2880. Some components of pump assembly 2882 are configured to move, while other components are designed to remain stationary with respect to reservoir 2880. A user initiates the operation of pump assembly 2882 by displacing moving frame 2830. Moving frame 2830 may optionally include one or more finger holds 2831. The embodiment shown in FIG. 28 includes two finger holds. Moving frame may also optionally include a tip portion 2892 that may provide a comfortable fit or feel when inserted into the nasal cavity. In some embodiments, tip portion 2892 may be formed of a soft, resilient material. Tip portion 2892 may be attached to moving frame 2830, and may move with moving frame 2830.

Moving frame 2830 may include an internal conduit 2894. Internal conduit 2894 may be connected to actuator 2832. The connection between internal conduit 2894 and actuator 2832 may allow actuator 2832 to move with moving frame 2830. In the embodiment shown in FIG. 28, internal conduit 2894 includes a female mechanical connector, while actuator 2832 includes a mating male mechanical connector. However, any suitable connector may be used.

In the embodiment shown in FIG. 28, actuation spring 2834 is disposed coaxially, and radially outward over a portion of actuator 2832. Actuation spring 2834 is also axially disposed between a shoulder of actuator 2832 and piston 2838. Actuation spring 2834 may help to return moving member 2830 to its original spring biased rest position. Actuation spring 2834 can also allow a little bit of axial travel before actuation spring 2834 bottoms out and actuator 2832 begins to move piston 2838.

As actuator 2832 continues to move piston 2838, piston 2838 begins to move valve 2842 away from valve seat 2840. Piston 2838 also moves against the biasing force of main spring 2844. A portion of actuator 2832 and piston 2838 move within pump housing 2846. The lower portion of pump housing 2846 includes an entry orifice 2850. Check valve 2848 is disposed proximate orifice 2850, and check valve 2848 cooperates with orifice 2850. Pump housing 2846 is generally stationary compared to actuator 2832 and piston 2838, and may be attached to reservoir 2880. In the embodiment shown in FIG. 28, a retaining clip 2836 is used to hold pump housing 2846 in place, and to attach pump housing 2846 to reservoir 2880. In some embodiments, retaining clip 2836 may include joint packing or seal 2890.

As actuator 2832 continues to move piston 2838 towards orifice 2850, a compression chamber is formed between piston 2838 and check valve 2850. Piston 2838 includes a fluid tight seal between its outer diameter and the inner diameter of pump housing 2846. At the opposite end of the compression chamber, check valve 2850 allows fluid to pass from the entry orifice 2850 to the compression chamber, but check valve 2850 prevents the reverse flow, from the compression chamber back out of orifice 2850.

By preventing this back flow, continued motion of piston 2838 towards orifice 2850 builds pressure within the compression chamber. This increased pressure eventually urges fluid trapped in the compression chamber to move past the gap formed between valve 2842 and valve seat 2840. The fluid will continue to move up through a central passageway formed in actuator 2832, then into a second central passageway formed in the internal conduit 2894 of moving frame 2830. The fluid continues to rise through the second central passageway, and eventually is dispensed through tip aperture 2896 formed in tip 2892.

After fluid has been dispensed, the user relaxes their fingers and moving frame 2830 moves away from reservoir 2880. This upward motion also creates a vacuum within the compression chamber as piston 2838 moves away from orifice 2850. This vacuum effect may draw drug formulation 2884 through dip tube 2852, past orifice 2850, and into the compression chamber. By pre-loading the compression chamber, pump assembly 2882 is ready for the next actuation event.

Some embodiments may include a second pump assembly 2982. Generally, second pump assembly 2982 may be substantially similar to pump assembly 2882, however, second pump assembly 2982 may be configured to dispense a different amount of drug formulation 2884 as pump assembly 2882. In some cases, second pump assembly 2982 is designed to deliver a smaller quantity of drug formulation 2884 as pump assembly 2882. The operation of the two pump assemblies may be related. In one embodiment, the pump assemblies may be operated cooperatively.

Like pump assembly 2882, second pump assembly 2982 can include provisions to dispense precise quantities of drug formulation 2884 contained within reservoir 2880. Some components of second pump assembly 2982 are configured to move, while other components are designed to remain stationary with respect to reservoir 2880.

As the user initiates the operation of the pump assemblies by displacing moving frame 2830, moving frame 2830 in turn moves actuator 2832. In those embodiments that include a second pump assembly 2982, a bridge 2900 may be provided that mechanically link actuator 2832 with second actuator 2932. Bridge 2900 can allow the two actuators move in unison.

In the embodiment shown in FIG. 28, second actuation spring 2934 is disposed coaxially, and radially outward over a portion of second actuator 2932. Second actuation spring 2934 is also axially disposed between a shoulder of second actuator 2932 and second piston 2938. Second actuation spring 2934 can allow a little bit of axial travel before second actuation spring 2934 bottoms out and second actuator 2932 begins to move second piston 2938.

As second actuator 2932 continues to move second piston 2938, second piston 2938 begins to move second valve 2942 away from second valve seat 2940. Second piston 2938 also moves against the biasing force of second main spring 2944. A portion of second actuator 2932 and second piston 2938 move within second pump housing 2946. The lower portion of second pump housing 2946 includes a second entry orifice 2950. Second check valve 2948 is disposed proximate second orifice 2950, and second check valve 2948 cooperates with second orifice 2950. Second pump housing 2946 is generally stationary compared to second actuator 2932 and second piston 2938, and may be attached to reservoir 2880. In the embodiment shown in FIG. 28, a retaining clip 2836 is used to also hold second pump housing 2946 in place, and to attach second pump housing 2946 to reservoir 2880.

As second actuator 2932 continues to move second piston 2938 towards second orifice 2950, a second compression chamber is formed between second piston 2938 and second check valve 2950. Second piston 2938 includes a fluid tight seal between its outer diameter and the inner diameter of second pump housing 2946. At the opposite end of the second compression chamber, second check valve 2950 allows fluid to pass from the second entry orifice 2950 to the second compression chamber, but second check valve 2950 prevents the reverse flow, from the second compression chamber back out of second orifice 2950.

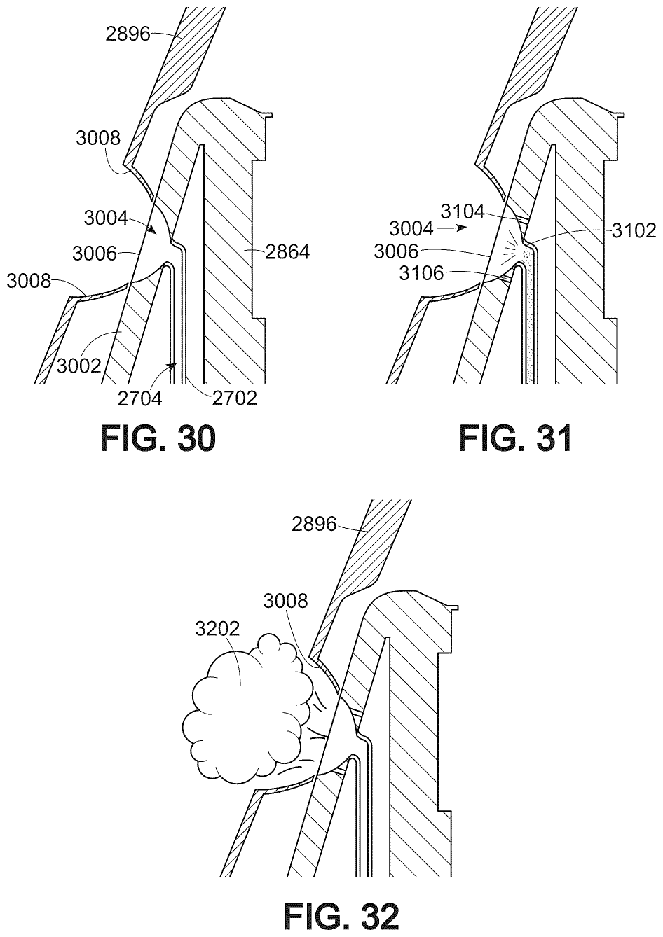

By preventing this back flow, continued motion of second piston 2938 towards second orifice 2950 builds pressure within the second compression chamber. This increased pressure eventually urges fluid trapped in the second compression chamber to move past the gap formed between second valve 2942 and second valve seat 2940. The fluid will continue to move up through a central passageway formed in second actuator 2932, then into feed tube 2702. Feed tube 2702 directs fluid towards a lateral portion of tip portion 2892. In some embodiments, tip portion 2892 may include a recessed portion 2704, shown in the enlarged FIG. 30.

After fluid has been dispensed, the user relaxes their fingers and moving frame 2830 moves away from reservoir 2880. This upward motion also creates a vacuum within the second compression chamber as second piston 2938 moves away from second orifice 2950. This vacuum effect may draw drug formulation 2884 through second dip tube 2952, past second orifice 2950, and into the second compression chamber. By pre-loading the second compression chamber, second pump assembly 2982 is ready for the next actuation event.

The two pump assemblies may be configured to provide different fluid flow characteristics, even though the two pump assemblies are operated together. In some embodiments, the second pump assembly 2982 may be designed to pump a smaller quantity of fluid than pump assembly 2882. In some cases, the difference in pumped fluid volume between the two pump assemblies may be characterized as a percentage of the total pumped volume of the two pump assemblies combined. In one embodiment, the second pump assembly 2982 may pump between about 5 to 50% of the total volume, while pump assembly may pump between about 95% to 50% of the total pumped volume. In another embodiment, second pump assembly 2982 may pump between 10 to 40% of the total volume, while pump assembly 2982 may pump between 90 to 60% of the total pumped volume. In yet another embodiment, second pump assembly 2982 may pump between 15 to 25% of the total pumped volume, while pump assembly 2883 may pump between 85 to 75% of the total pumped volume. In an exemplary embodiment, second pump assembly 2982 may pump around 20% of the total pumped volume, while pump assembly 2883 may pump around 80% of the total pumped volume.

The differences between the two pump assemblies may be configured by modifying the size or diameter of the pump assembly. The size and configuration of each compression chamber of each pump assembly compared to the other compression chamber may be modified to achieve the desired pump volume percentage or ratio.

In some embodiments, second dip tube 2952 may be associated with dip tube 2852. In the embodiment shown in FIG. 28, second dip tube 2952 is attached to dip tube 2852 along a portion of their lower length, see FIG. 29, so that both dip tubes can draw drug formulation 2884 from a lower portion of reservoir 2880. The dip tubes separate at their upper portion to attach to their respective pump housings.

Returning to feed tube 2702, and with reference to FIGS. 30-32, the operation of a lateral aperture will now be described. Recall from FIG. 28 that FIG. 30 is an enlarged view of a lateral portion of tip portion 2896 and conical portion 3002 of moving frame 2830. Internal conduit 2894 is also shown for reference. Feed tube 2702 is attached to conical portion 3002 at a desired lateral location. Conical portion 3002 includes an expansion chamber 3004. Feed tube 2702 is attached to conical portion 3002 so that an inner fluid channel 2704 is in fluid communication with expansion chamber 3004. Expansion chamber 3004 includes a microgrid 3006 that substantially covers the expansion chamber 3004 along the outer wall of conical portion 3002.

In some embodiments, tip portion 2896 may include a recess 3008 that corresponds with expansion chamber 3004 formed on the conical portion 3002. In the embodiment shown in FIGS. 30-32, recess 3008 includes a contour or shape that substantially matches the shape of expansion chamber 3004, thus providing a nearly continuous shape. In other embodiments, tip portion 2896 may just have a hole or a shape that does not match the contour of expansion chamber 3004.

Referring to FIG. 31, as fluid is drawn towards conical portion 3002, the fluid eventually exits lateral orifice 3102 and enters expansion chamber 3004. In the embodiment shown in FIG. 31, expansion chamber 3004 has a larger diameter than lateral orifice 3102. The difference in diameter between the expansion chamber 3004 and lateral orifice 3102 may be selected to produce the desired flow characteristics. In the embodiment shown in FIG. 31, the diameter difference between expansion chamber 3004 and lateral orifice 3102 has been selected to distribute the fluid over a larger surface area and to rapidly reduce the velocity of the fluid.

Additional optional features may be provided that assist in modifying the fluid flow. In some embodiments, lateral orifice 3102 may include a fluid flow regulator, restrictor, or sprayer nozzle to help distribute the fluid or reduce the velocity of the fluid. Some embodiments may include additional apertures that can be used to modify the fluid flow within expansion chamber 3004. These apertures may be distributed throughout expansion chamber 3004. In the embodiment shown in FIG. 31, two of the four optional apertures are shown. First aperture 3104 may be disposed further away from feed tube 2702, while second aperture 3106 is disposed closer to feed tube 2702. There may be two additional apertures so that four apertures are evenly distributed around lateral orifice 3102. These apertures may be used to modify the fluid flow of the fluid in expansion chamber 3004. In some cases, these apertures provide a pressure relief region that can further distribute fluid within expansion chamber 3004 or can further slow the velocity of the fluid in expansion chamber 3004.

Eventually, the fluid is distributed over microgrid 3006 disposed along the outer wall of conical portion 3002. The size and configuration of microgrid 3006 may be selected to achieve the desired spray pattern, droplet size and velocity of the fluid exiting the device. Any desired microgrid or grid assembly may be used, including the embodiments disclosed above in connection with FIGS. 20A-21E. In some embodiments, tip portion 2896 may include recess 3008 that has a shape that further assists in achieving the desired fluid flow characteristics.

After passing through microgrid 3006, the fluid eventually moves past conical portion 3002. In the embodiment shown in FIG. 32, a mist 3202 containing micron sized fluid droplets has been formed. Mist 3202 also has a velocity and pressure profile that has been selected for optimal drug delivery to a subject's VNO. Portions of mist 3202 may travel away from tip portion 2896 and towards selected anatomical features within the nasal cavity, in some cases, the VNO.

In the context of the intricate pathophysiology of brain disorders and increasingly complex, high-risk and costly drug development, difficulty in delivering therapeutics into the CNS has represented a major hurdle for new CNS therapies. As described herein, the various locations of the anatomical features and potential target sites for drug delivery can be used to determine an optimal spray pattern and output, which can then be used to determine the appropriate actuator configuration and discharge orifice characteristics that can help fine-tune the delivery of drugs. Rapid direct drug transport along the olfactory and trigeminal nerves allows brain access for small and large molecules and even stem cells in therapeutic concentrations (‘nose to brain’ or N2B). For non-pherine compounds, the disclosed embodiments offer significant benefits and improvements over conventional intranasal devices, allowing for dispensation of compounds at highly-focused or specialized regions in the nostril, thereby overcoming the challenges associated with the relatively small dimensions of the nasal valve and its irregular and narrow triangular shape. The proposed embodiments therefore substantially improve the efficacy of deposition of fluid at specific sites in the nasal cavities, in particular at secondary chemosensory sites.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some examples be excised from the combination, and the claimed combination may be directed to a sub-combination or variation of a sub-combination. While various embodiments are described, the description is intended to be exemplary rather than limiting, and it will be apparent to those of ordinary skill in the art that many more embodiments and implementations are possible that are within the scope of the disclosure. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted. Also, various modifications and changes may be made within the scope of the attached claims. Accordingly, the embodiments are not to be restricted except in light of the attached claims and their equivalents. Further, unless otherwise specified, any step in a method or function of a system may take place in any relative order in relation to any other step described herein.

We claim:

1. A method of delivering drugs to multiple regions of a subject's nasal cavity including the olfactory cleft and the vomeronasal organ (VNO), the method comprising:
inserting a distal end of an actuator of a delivery device through a first nostril and into a first nasal cavity of a person until:
a distal orifice formed at an outermost tip of the distal end is between about 35 mm and 45 mm from the first nostril; and
a first lateral orifice formed in a first portion of a sidewall of the actuator is between about 15 mm and 25 mm from the first nostril.

2. The method of claim 1, further comprising positioning the delivery device so that a flange attached to a lowermost portion of the actuator abuts the first nostril.

3. The method of claim 1, further comprising delivering, to the VNO, a first metered dose of a first pherine compound via the first lateral orifice.

4. The method of claim 1, further comprising orienting the actuator so that the first lateral orifice is facing toward a septum of the first nasal cavity.

5. The method of claim 1, wherein the first portion is recessed relative to a second portion of the sidewall that surrounds the first portion.

6. A method of delivering drugs to multiple regions of a subject's nasal cavity including the vomeronasal organ (VNO), the method comprising:
delivering a first metered dose with an impact pressure of no more than 0.9 Pascals via a lateral orifice provided in an actuator body of a delivery device to the VNO of a first nasal cavity, thereby covering surfaces of the vomeronasal organ of the first nasal cavity while preventing activation of a majority of high threshold trigeminal mechanoreceptors in the first nasal cavity.

7. The method of claim 6, further comprising delivering a second metered dose with an impact pressure of no more than 0.9 Pascals via a distal orifice formed at an outermost tip of the distal end to an olfactory cleft region of a first nasal cavity, thereby covering surfaces of an olfactory mucosa region of the first nasal cavity while preventing activation of a majority of high threshold trigeminal mechanoreceptors in the first nasal cavity.

8. The method of claim 7, wherein the first metered dose is emitted as a mist, and the second metered dose is emitted as a plume.

9. The method of claim 7, wherein a volume of the first metered dose is less than a volume of the second metered dose.

10. The method of claim 6, wherein the lateral orifice comprises a plurality of micro-pores.

11. A method for treating a disorder, the method comprising:
intranasally administering to an individual in need thereof an effective dose of a pherine compound;
wherein the pherine compound is delivered to a first region of a nasal cavity that includes the vomeronasal organ (VNO).

12. The method of claim 11, wherein the pherine compound is selected from one of fasedienol, itruvone, PH80, PH15, and PH284.

13. The method of claim 11, wherein the disorder is one of social anxiety, separation anxiety, generalized anxiety, obsessive-compulsive symptoms, sound phobias, dysmenorrhea, and depression.

14. The method of claim 11, wherein the pherine compound is also delivered to a second region of the nasal cavity that includes the olfactory cleft.

15. The method of claim 11, wherein the pherine compound is delivered to the first region as a mist.

16. An intranasal nasal drug delivery device comprising:
a reservoir that includes a pherine composition selected from the group consisting of one or more of fasedienol, itruvone, PH80, PH15, and PH284; and
an actuator in fluid communication with contents of the reservoir, the actuator including an actuator body comprising of a tubular sidewall extending from a base portion to a distal tip portion, wherein:
the distal tip portion includes a distal orifice configured to direct a first portion of the pherine composition onto nasal chemosensory receptors associated with the mucosa in the olfactory cleft, and
the tubular sidewall includes a lateral orifice configured to direct a second portion of the pherine composition onto nasal chemosensory receptors associated with the vomeronasal organ (VNO).

17. The intranasal nasal drug delivery device of claim 16, wherein the actuator body includes an inferior portion and a superior portion, and the inferior portion includes a substantially D-shaped cross-section that constrains insertion of the actuator into a nasal cavity to one of two orientations.

18. The intranasal nasal drug delivery device of claim 16, wherein an elasticity of a material comprising the actuator body decreases gradually in a direction extending from the distal tip portion to the base portion.

19. The intranasal nasal drug delivery device of claim 16, wherein the lateral orifice comprises a plurality of micro-pores configured to generate a mist when emitting the pherine composition.

20. The intranasal nasal drug delivery device of claim 16, wherein the lateral orifice emits pherine composition diagonally at a first spray angle that is between 10 degrees and 80 degrees relative to a vertical axis of the actuator body.
